(19) United States
(12) Patent Application Publication
(10) Pub. No.: US 2005/0014711 A1
(43) Pub. Date: Jan. 20, 2005

(54) Composition, formulations & methods for prevention and treatment of diseases and conditions associated with bronchoconstriction, allergy(ies) and inflammation

(76) Inventor: Jonathan W. Nyce, Princeton, NJ (US)

Correspondence Address: WILSON SONSINI GOODRICH & ROSATI, 650 PAGE MILL ROAD, PALO ALTO, CA, 943041050, US

(21) Appl. No.: 10/758,451

(22) Filed: Jan. 14, 2004

Related U.S. Application Data
(63) Continuation of application No. 09/093,972, filed on Jun. 9, 1998.

(51) Int. Cl. A61K 48/00

(52) U.S. Cl. 514/44

(57) ABSTRACT

A pharmaceutical composition effective for preventing and alleviating bronchoconstriction, allergy(ies) and/or inflammation comprises a surfactant and a nucleic acid comprising an oligonucleotide anti-sense to an adenosine A₁, A₂ₐ, A_{2b} or A₃ receptor gene, mRNA, flanking regions or regions bridging the intro/exon borders, analogues which bind thymidine but have low adenosine content or exhibit lower or no adenosine receptor agonist activity, combinations thereof, physiologically acceptable salts thereof or mixtures thereof. The composition is formulated for administration by a multiplicity of routes for the prevention or alleviation of diseases and conditions associated with breathing difficulties, impeded and obstructed airways, bronchoconstriction, allergy and/or inflammation. Among the applications of this technology are the prevention and treatment of diseases and conditions such as asthma, kidney damage or failure, ARDS, pulmonary vasoconstriction, inflammation, allergies, impeded respiration, respiratory distress syndrome, pain, cystic fibrosis, pulmonary hypertension, pulmonary vasoconstriction, emphysema, chronic obstructive pulmonary disease (COPD), and cancers such as leukemias, lymphomas, carcinomas, and the like.

This invention was made at least partially with United States Government support under grant RO1CA47217-06 from the National Cancer Institute. The Government may have certain rights to this invention.

### BACKGROUND OF THE INVENTION

1. Field of the Invention

This invention relates to compositions and formulations of oligonucleotides and surfactants, which are highly effective for the prevention and treatment of diseases and conditions associated with difficult breathing, bronchoconstriction, impeded airways, allergy(ies) and inflammation of the lungs.

2. Description of the Background

Adenosine A₁-mediated diseases and conditions, such as asthma and Acute Respiratory Distress Syndrome (ARDS), among others, are common diseases in industrialized countries, and in the United States alone account for extremely high health care costs. These diseases or conditions have recently been increasing at an alarming rate, both in terms of prevalence and mortality. Occupational asthma is predicted to be the preeminent occupational lung disease in the next decade. In many of these, the underlying causes remain poorly understood.

Adenosine, a natural nucleoside, may constitute an important natural mediator of bronchial asthma and ARDS. The potential role of adenosine in these diseases or conditions is supported by experimental findings that, for example and in contrast to normal individuals, asthmatics respond to aerosolized adenosine with marked bronchoconstriction. Similarly, asthmatic rabbits produced using the dust mite allergic rabbit model of human asthma also were shown to respond to aerosolized adenosine with marked bronchoconstriction, while non-asthmatic rabbits showed no response. Recent work using this model system has suggested that adenosine-mediated bronchoconstriction in asthma is mediated through the stimulation of the adenosine A₁ receptor. Other experimental data suggest the possibility that adenosine receptors may also be involved in allergic and inflammatory responses.

Adenosine receptor antagonists, such as theophylline, are known to counter adenosine-mediated bronchoconstriction in asthmatic rabbits. Pre-treatment with another adenosine A₁-specific receptor antagonist, 8-cyclopentyl-1,3-dipropylxanthine (DPCPX), also inhibited adenosine-mediated bronchoconstriction and bronchial hyper-responsiveness in an allergic rabbit model. The therapeutic potential, however, of currently available adenosine A₁ receptor-specific antagonists is drastically limited by their toxicity. Theophylline, for example, although widely used in the treatment of asthma, may result in frequent and significant toxicity because of its narrow therapeutic dose range.

The availability of a novel strategy to prevent and/or counter adenosine receptor-associated effects of disorders and conditions associated with symptoms such as pulmonary bronchoconstriction, impeded respiration, inflammation and allergy(ies), among others, of great practical importance. Such technology is clearly applicable to the treatment of ailments including Acute Respiratory Disorder Syndrome (ARDS), asthma, respiratory distress syndrome, pain, cystic fibrosis, pulmonary hypertension, pulmonary vasoconstriction, emphysema, chronic obstructive pulmonary disease (COPD), and cancers such as leukemias, lymphomas, carcinomas, and the like, including colon cancer, breast cancer, lung cancer, pancreatic cancer, hepatocellular carcinoma, kidney cancer, melanoma, hepatic metastases, etc., as well as all types of cancers which may metastasize or have metastasized to the lung(s), including breast and prostate cancer would clearly find an immediate therapeutic application. Similarly, a composition and method which are suitable for administration before, during and after other treatments, including radiation, chemotherapy, antibody therapy, phototherapy and cancer, and other types of surgery, and that may be effectively administered preventatively, prophylactically or therapeutically, and in conjunction with other therapies, or by itself for conditions without known therapies or as a substitute for therapies that have significant negative side effects is also of immediate clinical application.

### SUMMARY OF THE INVENTION

The present invention relates to a pharmaceutical composition effective to alleviate bronchoconstriction, allergy and/or inflammation, comprising a surfactant, and an anti-adenosine A₁, A_{2b} or A₃ receptor or anti-adenosine A₂ₐ receptor oligonucleotide exhibiting at least some adenosine A₁, A_{2b} or A₃ receptor inhibitory activity, analogues thereof which bind to thymidine but evidence either reduced adenosine content or reduced adenosine receptor activating activity, combinations thereof, physiologically acceptable salts thereof or mixtures thereof. The composition of this invention may be formulated for administration by various different routes, such as topical and systemic, e.g. oral, parenteral, inhalable, and the like, and are generally administered in amounts which prevent or reduce adenosine receptor associated side effects such as bronchoconstriction, allergy(ies), inflammation and airway obstruction, among others. The present compositions and formulations, thus, are suitable for the prevention and alleviation of adenosine receptor associated bronchoconstriction, allergy and/or inflammation and, therefore, in the treatment of Acute Respiratory Disorder Syndrome (ARDS), asthma, side effects associated with adenosine administration in SupraVentricular Tachycardia (SVT) and in stress tests to hyper-sensitized individuals, ischemia, renal damage or failure induced by certain drugs, respiratory distress syndrome, pain, cystic fibrosis, pulmonary hypertension, pulmonary vasoconstriction, emphysema, chronic obstructive pulmonary disease (COPD), and cancers such as leukemias, lymphomas, carcinomas, and the like, including colon cancer, breast cancer, lung cancer, pancreatic cancer, hepatocellular carcinoma, kidney cancer, melanoma, hepatic metastases, etc., as well as all types of cancers which may metastasize or have metastasized to the lung(s), including breast and prostate cancer, among others. The present technology is also applicable in conjunction with other procedures and/other therapies, including other therapeutic agents such as antibody therapy and chemotherapy, among others, radiation, phototherapy, and cancer and other types of surgery, and is effectively administered preventatively, prophylactically or therapeutically.

### BRIEF DESCRIPTION OF THE DRAWINGS

FIG. 1 illustrates the effects of A₁ adenosine receptor anti-sense oligonucleotides and mismatch control anti-sense oligonucleotides on the dynamic compliance of the bronchial airway in a rabbit model. The two stars represent significant difference at p<0.01, Student's t-test.

FIG. 2 illustrates the specificity of A₁ adenosine receptor anti-sense oligonucleotides as indicated by the A₁ and A₂ adenosine receptor number present in airway tissue treated with A₁ adenosine receptor anti-sense oligonucleotides.

FIGS. 3a and 3b illustrate the response of two hyper-responsive monkeys (ascaris sensitive) to a challenge with inhaled adenosine. The right hand bar represents the PC₄₀ adenosine after administration of the Oligo I, whereas the left hand bar represents the PC₄₀ adenosine value prior to treatment with the Oligo I. The PC₄₀ adenosine, represented in the Y axis, is the amount of adenosine in mg that causes a 40% decrease in dynamic compliance in hyper-responsive airways.

FIG. 3a represents the experimental results obtained without and with pre-treatment of a first monkey with a phosphorothioate agent of the invention (anti-sense oligo I; SEQ. ID NO:1), prior to administration of adenosine.

FIG. 3b represents the experimental results obtained without and with pre-treatment of a second monkey with a phosphorothioate agent of this invention (anti-sense oligo I; SEQ. ID NO:1), prior to administration of adenosine.

### DETAILED DESCRIPTION OF THE PREFERRED EMBODIMENTS

This invention arose from a desire by the inventor to improve on his own prior technology for the treatment of acute bronchoconstriction, allergy and/or inflammation associated with various diseases and conditions, including Acute Respiratory Distress Syndrome (ARDS), asthma, adenosine administration e.g. in the treatment of SupraVentricular Tachycardia (SVT) and other arrhythmias, and in stress tests to hyper-sensitized individuals, ischemia, renal damage or failure induced by certain drugs, infantile respiratory distress syndrome, pain, cystic fibrosis, pulmonary hypertension, pulmonary vasoconstriction, emphysema, chronic obstructive pulmonary disease (COPD), and cancers such as leukemias, lymphomas, carcinomas, and the like, including colon cancer, breast cancer, lung cancer, pancreatic cancer, hepatocellular carcinoma, kidney cancer, melanoma, hepatic metastases, etc., as well as all types of cancers which may metastasize or have metastasized to the lung(s), including breast and prostate cancer. The inventor, in addition, wanted to provide a treatment which would improve the outcome and life stile of patients undergoing other procedures or being administered other therapies, including antibody therapy, chemotherapy, radiation, phototherapy, and surgery e.g. cancer surgery, and that could be effectively administered preventatively, prophylactically or therapeutically.

He succeeded in this endeavor and is providing in this patent novel and improved compositions, formulations and methods which afford greatly improved results when compared with previously known treatments for preventing and alleviating bronchoconstriction, allergy(ies), inflammation, breathing difficulties and blockage of airways. The nucleic acid and surfactant components of the bare bone composition of the invention may be formulated alone with a carrier, or with other therapeutic agents and formulation agents as is known in the art. The compositions of this invention, thus, may be incorporated into a variety of formulations for systemic and topical administration.

In the past, anti-sense oligonucleotides received considerable theoretical consideration as being potentially useful as pharmacologic agents for the treatment of human disease. R. Wagner, Nature 372: 333-335 (1994). However, it has been difficult to actually apply these molecules to alleviating and curing human diseases. One important consideration in the pharmacologic application of these molecules has been the failure of various routes of administration to deliver the compounds to its target while avoiding invading the circulation and, therefore, other untargeted tissues which, thus, produces a plethora of side effects. Most in vivo experiments utilizing anti-sense oligonucleotides involved a direct application of the oligo to limited regions of the brain. See, C. Wahlestedt, Trends in Pharmacol. Sci. 15: 42-46 (1994); J. Lai et al., Neuroreport 5: 1049-1052 (1994); K. Standifer et al., Neuron 12: 805-810 (1994); A. Akabayashi et al., Brain Res. 21: 55-61 (1994). Others applied them into the spinal fluid. See, e.g. L. Tseng et al., European J. Pharmacol. 258: R1-3 (1994); R. Raffa et al., European J. Pharmacol. 258: R5-7 (1994); F. Gillardon et al., European J. Neurosci. 6: 880-884 (1994). Such applications, clearly, have no practical clinical utility due to their invasive nature. Thus, the systemic administration of anti-sense oligonucleotides poses significant problems with respect to their pharmacologic application, not the least of which is the difficulty in selectively targeting disease-involved tissues.

The systemic administration of anti-sense oligonucleotides also poses significant problems with respect to their pharmacologic application, not the least of which is the difficulty in selectively targeting disease-involved tissues. The respiratory system, and in particular the lung, as the ultimate port of entry into the organism, however, is an excellent route of administration for anti-sense oligonucleotides. This is so not only for the treatment of lung disease, but also when utilizing the lung as a means for delivery, particularly because of its non-invasive and tissue-specific nature. Thus, local delivery of antisense oligonucleotides directly to the target tissue enables the therapeutic use of these compounds. Fomivirsen (ISIS 2302) is an example of a local drug delivery into the eye to treat cytomegalovirus (CMV) retinitis, for which a new drug application has been filed by ISIS. The administration of a drug through the lung offers the further advantage that inhalation is non-invasive whereas direct injection in to the vitreous of the eye is invasive.

The composition and formulations of this invention have been shown to have an exceedingly high efficacy for preventing and treating a disease or condition associated with bronchoconstriction, difficult breathing, impeded and obstructed lung airways, allergy(ies) and/or inflammation. The examples provided below show a complete inhibition of such adenosine receptor associated symptoms in a rabbit model for human bronchoconstriction, allergy(ies) and inflammation as well as the elimination of the ability of the adenosine receptor agonist par excellence, adenosine, to cause bronchoconstriction in hyper-responsive monkeys, which are animal models for human hyper-responsiveness to adenosine receptor agonists. The pharmaceutical composition and formulations of the invention, therefore, are suitable for preventing and alleviating the symptoms associated with stimulation of adenosine receptors, such as the adenosine A₁ receptors. The compositions and formulations of this invention, thus, are also suitable for prevent the untoward side effects of adenosine-mediated hyperresponsiveness in certain individuals, which are generally seen in diseases affecting respiratory activity. Examples of diseases and conditions, which may be treated preventatively, prophylactically and therapeutically with the compositions and formulations of this invention, are pulmonary vasoconstriction, inflammation, allergies, asthma, impeded respiration, Acute Respiratory Distress Syndrome (ARDS), renal damage and failure associated with ischemia as well as the administration of certain drugs, side effects associated with adenosine administration e.g. in SupraVentricular Tachycardia (SVT) and in adenosine stress tests, infantile Respiratory Distress Syndrome (infantile RDS), pain, cystic fibrosis, pulmonary hypertension, pulmonary vasoconstriction, emphysema, chronic obstructive pulmonary disease (COPD), and cancers such as leukemias, lymphomas, carcinomas, and the like, e.g. colon cancer, breast cancer, lung cancer, pancreatic cancer, hepatocellular carcinoma, kidney cancer, melanoma, hepatic metastases, etc., as well as all other metastatic cancers, e.g. cancers which metastasized to the lung(s), breast and prostate. The present compositions and formulations are suitable for administration before, during and after other treatments, including radiation, chemotherapy, antibody therapy, phototherapy and cancer, and other types of surgery. The present compositions and formulations may also be administered effectively as a substitute for therapies that have significant negative side effects.

All nucleotide sequences are represented in this patent by a single strand only, and in the 5′ to 3′ direction, from left to right. All nucleotide and amino acids are represented in the manner recommended by the IUPAC-IUB Biochemical Nomenclature Commission, or (for amino acids) by three letter code, in accordance with 37 CFR § 1.822 and established usage. See, e.g., PatentIn User Manual, 99-102 (November 1990) (U.S. Patent and Trademark Office, Office of the Assistant Commissioner for Patents, Washington, D.C. 20231); U.S. Pat. No. 4,871,670 to Hudson et al. at col. 3, lines 20-43. The relevant sections of the disclosures of the above cited, and of all other patents and references cited in this patent are incorporated herein by reference.

The method of the present invention may be used to reduce adenosine receptor associated bronchoconstriction in the lungs of a subject for any reason, including, but not limited to, bronchoconstriction, allergy(ies) and/or inflammation. The compositions and formulations of the invention comprise a surfactant and an oligonucleotide which is anti-sense to the adenosine A₁, A_{2b} and A₃ receptors have shown to be effective in the down-regulation of the adenosine A₁, A_{2b} or A₃ receptors, respectively, in the cell. Others which are anti-sense to the adenosine A₂ₐ receptor are also effective as long as they have some adenosine A₁ inhibitory activity. One novel feature of this treatment, as compared to traditional treatments for adenosine-mediated bronchoconstriction and other symptoms, is that the compositions and formulations of this invention may be administered directly into the respiratory system of an individual, and even to his\her lungs. In addition, the present treatment may reduce the amount or level of a receptor protein itself rather than merely acting at the receptor as is the case with treatments and/or where the agent is merely an antagonist acting at the receptor site. The selective characteristic of the present compositions and formulations along with their administration by a selected route results in reduced toxicity.

As used herein, the terms “prevent”, “preventing”, “treat” or “treating” refer to a preventative or therapeutic treatment which decreases the likelihood that the subject administered such treatment will manifest symptoms associated with adenosine receptor stimulation. The term “down-regulate” refers to inducing a decrease in production, secretion or availability and, thus, a decrease in concentration, of intracellular adenosine A₁, A_{2b} or A₃ receptor or an increase in concentration of the adenosine A₂ₐ receptor. Although the present invention is primarily concerned with the treatment of human subjects, it is also applicable to the treatment of animals, such as other vertebrates, including mammals, large and small, wild and domesticated, including pets, e.g. dogs and cats, for veterinary purposes. In general, “anti-sense” refers to small, many times synthetic, oligonucleotides, resembling single-stranded DNA, targeted to a specific gene, its flanking regions, mRNA or protein encoded by the gene and mRNA, which may be utilized for inhibiting gene expression by inhibiting the function of the target messenger RNA (mRNA). Milligan, J. F. et al., J. Med. Chem. 36(14), 1923-1937 (1993). The present invention, thus, is intended for inhibiting gene expression of the adenosine A₁, A_{2b} or A₃ receptor as well as for promoting the gene expression of the adenosine A₂ₐ receptor. As is generally known in the art, the inhibition of gene expression may be I brought about through anti-sense oligonucleotide hybridization to the coding (sense) sequences in a specific messenger RNA (mRNA) target, e.g. by hydrogen bonding according to Watson-Crick base pairing rules. In general, the exogenously administered anti-sense oligos decrease the mRNA and protein levels of the target gene or cause changes in the growth characteristics or shapes of the cells. Ibid. See, also Helene, C. and Toulme, J., Biochim. Biophys. Acta 1049: 99-125 (1990); Cohen, J. S., Ed., Oligodeoxynucleotides as Antisense Inhibitors of Gene Expression; CRC Press: Boca Raton, Fla. (1987). As used herein, “adenosine receptor anti-sense oligonucleotide” is a short sequence of synthetic nucleotide that (1) hybridizes to any coding sequence in an mRNA which codes for an adenosine receptor, e.g., the adenosine A₁, A_{2b} or A₃ receptor, under in vivo hybridization conditions described below, and that (2) upon hybridization causes a decrease in gene expression of the adenosine A₁, A_{2b} or A₃ receptor.

The mRNA sequence of the adenosine A₁, A_{2b} and A₃ receptors may be derived from the DNA base sequences of the genes expressing either the adenosine A₁, A_{2b} and A₃ receptors. The sequence of the genomic human adenosine A₁ receptor is known and is disclosed in U.S. Pat. No. 5,320,962 to G. Stiles et al. The adenosine A_{2b} receptor is also known. See, GenBank, Accession No. X68486; GenBank Accession No. X68487. The adenosine A₃ receptor has been cloned, sequenced and expressed in rat and humans. See, F. Zhou et al., Proc. Nat'l. Acad. Sci. (USA) 89:7432 (1992); M. A. Jacobson et al., U.K. Patent Application No. 9304582.1 (1993). The antisense oligonucleotides that down-regulate the production of the adenosine A₁, A_{2b} and A₃ receptor may be produced in accordance with standard techniques.

The anti-sense agent of this invention binds specifically with any sequence of a mRNA molecule which encodes an adenosine A₁, A₂ₐ, A_{2b} or A₃ receptor, and prevents translation of the mRNA molecule. In one embodiment of the present invention, the anti-sense oligonucleotide has one of the following sequences. In another preferred embodiment, the agent of the invention comprises fragments of these sequences or their combinations as well as sequences with decreased adenosine contents when compared with the natural sequences, where one or more adenosines are replaced by a universal base or adenosine analogue which does not activate adenosine receptors, particularly adenosine A₁ receptors.

| | |
|---|---|
| 5′-GAT GGA GGG CGG CAT GGC GGG-3′ | (SEQ ID NO: 1) |
| 5′-GTT GTT GGG CAT CTT GCC-3′ | (SEQ ID NO: 3) |
| 5′-GTG GGC CTA GCT CTC GCC-3′ | (SEQ ID NO: 5) |

In another embodiment of the invention, the sequence of the anti-sense oligonucleotide brackets the initiation codon of the adenosine A₁ receptor, for example that of the human receptor mRNA. Preferred human adenosine A₁ receptor anti-sense oligonucleotide may have the SEQ. ID NO: 7 or any one of its fragments, including one of the following sequences. In another preferred embodiment, fragments of these sequences and/or their combinations are also within the confines of the invention.

| | |
|---|---|
| 5′-GGC GGC CTG GAA AGC TGA GAT | (SEQ. ID NO: 7) |
| GGA GGG CGG CAT GGC GGG GAG AGG | |
| CTG GGC-3′ | |
| 5′-GGC GGC CTG GAA AGC TGA GAT | (SEQ. ID NO: 8) |
| GGA GGG CGG CAT GGC GGG CAC AGG | |
| CTG GG-3′ (Fragment 1) | |
| 5′-GGC GGC CTG GAA AGC TGA GAT | (SEQ. ID NO: 9) |
| GGA GGG CGG CAT GGC GGG CAC AGG | |
| CTG G-3′ (Fragment 2) | |
| 5′-GGC GGC CTG GAA AGC TGA GAT | (SEQ. ID NO: 10) |
| GGA GGG CGG CAT GGC GGG CAC AGG | |
| CTG-3′ (Fragment 3) | |
| 5′-GGC GGC CTG GAA AGC TGA GAT | (SEQ. ID NO: 11) |
| GGA GGG CGG CAT GGC GGG CAC AGG | |
| CT-3′ (Fragment 4) | |
| 5′-GGC GGC CTG GAA AGC TGA GAT | (SEQ. ID NO: 12) |
| GGA GGG CGG CAT GGC GGG CAC AGG | |
| C-3′ (Fragment 5) | |
| 5′-GGC GGC CTG GAA AGC TGA GAT | (SEQ. ID NO: 13) |
| GGA GGG CGG CAT GGC GGG CAC | |
| AGG-3′ (Fragment 6) | |
| 5′-GGC GGC CTG GAA AGC TGA GAT | (SEQ. ID NO: 14) |
| GGA GGG CGG CAT GGC GGG CAC | |
| AG-3′ (Fragment 7) | |
| 5′-GGC GGC CTG GAA AGC TGA GAT | (SEQ. ID NO: 15) |
| GGA GGG CGG CAT GGC GGG CAC A-3′ | |
| (Fragment 8) | |
| 5′-GGC GGC CTG GAA AGC TGA GAT | (SEQ. ID NO: 16) |
| GGA GGG CGG CAT GGC GGG CAC-3′ | |
| (Fragment 9) | |
| 5′-GGC GGC CTG GAA AGC TGA GAT | (SEQ. ID NO: 17) |
| GGA GGG CGG CAT GGC GGG CA-3′ | |
| (Fragment 10) | |
| 5′-GGC GGC CTG GAA AGC TGA GAT | (SEQ. ID NO: 18) |
| GGA GGG CGG CAT GGC GGG C-3′ | |
| (Fragment 11) | |
| 5′-GGC GGC CTG GAA AGC TGA GAT | (SEQ. ID NO: 19) |
| GGA GGG CGG CAT GGC GGG-3′ | |
| (Fragment 12) | |
| 5′-GGC GGC CTG GAA AGC TGA GAT | (SEQ. ID NO: 20) |
| GGA GGG CGG CAT GGC GG-3′ | |
| (Fragment 13) | |
| 5′-GGC GGC CTG GAA AGC TGA GAT | (SEQ. ID NO: 21) |
| GGA GGG CGG CAT GGC G-3′ | |
| (Fragment 14) | |
| 5′-GGC GGC CTG GAA AGC TGA GAT | (SEQ. ID NO: 22) |
| GGA GGG CGG CAT GGC-3′ | |
| (Fragment 15) | |
| 5′-GGC GGC CTG GAA AGC TGA GAT | (SEQ. ID NO: 23) |
| GGA GGG CGG CAT GG-3′ | |
| (Fragment 16) | |
| 5′-GGC GGC CTG GAA AGC TGA GAT | (SEQ. ID NO: 24) |
| GGA GGG CGG CAT G-3′ | |
| (Fragment 17) | |
| 5′-GGC GGC CTG GAA AGC TGA GAT | (SEQ. ID NO: 25) |
| GGA GGG CGG CAT-3′ (Fragment 18) | |
| 5′-GGC GGC CTG GAA AGC TGA GAT | (SEQ. ID NO: 26) |
| GGA GGG CGG CA-3′ (Fragment 19) | |
| 5′-GGC GGC CTG GAA AGC TGA GAT | (SEQ. ID NO: 27) |
| GGA GGG CGG C-3′ (Fragment 20) | |
| 5′-GGC GGC CTG GAA AGC TGA GAT | (SEQ. ID NO: 28) |
| GGA GGG CGG-3′ (Fragment 21) | |
| 5′-GGC GGC CTG GAA AGC TGA GAT | (SEQ. ID NO: 29) |
| GGA GGG CG-3′ (Fragment 22) | |
| 5′-GGC GGC CTG GAA AGC TGA GAT | (SEQ. ID NO: 30) |
| GGA GGG C-3′ (Fragment 23) | |
| 5′-GGC GGC CTG GAA AGC TGA GAT | (SEQ. ID NO: 31) |
| GGA GGG-3′ (Fragment 24) | |
| 5′-GGC GGC CTG GAA AGC TGA GAT | (SEQ. ID NO: 32) |
| GGA GG-3′ (Fragment 25) | |
| 5′-GGC GGC CTG GAA AGC TGA GAT | (SEQ. ID NO: 33) |
| GGA G-3′ (Fragment 26) | |
| 5′-GGC GGC CTG GAA AGC TGA GAT | (SEQ. ID NO: 34) |
| GGA-3′ (Fragment 27) | |
| 5′-GGC GGC CTG GAA AGC TGA GAT | (SEQ. ID NO: 35) |
| GG-3′ (Fragment 28) | |
| 5′-GGC GGC CTG GAA AGC TGA GAT | (SEQ. ID NO: 36) |
| G-3′ (Fragment 29) | |
| 5′-GGC GGC CTG GAA AGC TGA GAT- | (SEQ. ID NO: 37) |
| 3′ (Fragment 30) | |
| 5′-GGC GGC CTG GAA AGC TGA GA-3′ | (SEQ. ID NO: 38) |
| (Fragment 31) | |
| 5′-GGC GGC CTG GAA AGC TGA G-3′ | (SEQ. ID NO: 39) |
| (Fragment 32) | |
| 5′-GGC GGC CTG GAA AGC TGA-3′ | (SEQ. ID NO: 40) |
| (Fragment 33) | |
| 5′-GGC GGC CTG GAA AGC TG-3′ | (SEQ. ID NO: 41) |
| (Fragment 34) | |
| 5′-GGC GGC CTG GAA AGC T-3′ | (SEQ. ID NO: 42) |
| (Fragment 35) | |
| 5′-GGC GGC CTG GAA AGC-3′ | (SEQ. ID NO: 43) |
| (Fragment 36) | |
| 5′-GGC GGC CTG GAA AG-3′ | (SEQ. ID NO: 44) |
| (Fragment 37) | |
| 5′-GGC GGC CTG GAA A-3′ | (SEQ. ID NO: 45) |
| (Fragment 38) | |
| 5′-GGC GGC CTG GAA-3′ | (SEQ. ID NO: 46) |
| (Fragment 39) | |
| 5′-GGC GGC CTG GA-3′ | (SEQ. ID NO: 47) |
| (Fragment 40) | |
| 5′-GGC GGC CTG G-3′ | (SEQ. ID NO: 48) |
| (Fragment 41) | |
| 5′-GC GGC CTG GAA AGC TGA GAT | (SEQ. ID NO: 49) |
| GGA GGG CGG CAT GGC GGG CAC AGG | |
| CTG GGC-3′ (Fragment 42) | |
| 5′-GC GGC CTG GAA AGC TGA GAT | (SEQ. ID NO: 50) |
| GGA GGG CGG CAT GGC GGG CAC AGG | |
| CTG GG-3′ (Fragment 43) | |
| 5′-GC GGC CTG GAA AGC TGA GAT | (SEQ. ID NO: 51) |
| GGA GGG CGG CAT GGC GGG CAC | |
| AGG CTG G-3′ (Fragment 44) | |
| 5′-GC GGC CTG GAA AGC TGA GAT | (SEQ. ID NO: 52) |
| GGA GGG CGG CAT GGC GGG CAC AGG | |
| CTG-3′ (Fragment 45) | |
| 5′-GC GGC CTG GAA AGC TGA GAT | (SEQ. ID NO: 53) |
| GGA GGG CGG CAT GGC GGG CAC AGG | |
| CT-3′ (Fragment 46) | |
| 5′-GC GGC CTG GAA AGC TGA GAT | (SEQ. ID NO: 54) |
| GGA GGG CGG CAT GGC GGG CAC AGG | |
| C-3′ (Fragment 47) | |
| 5′-GC GGC CTG GAA AGC TGA GAT | (SEQ. ID NO: 55) |
| GGA GGG CGG CAT GGC GGG CAC | |
| AGG-3′ (Fragment 48) | |
| 5′-GC GGC CTG GAA AGC TGA GAT | (SEQ. ID NO: 56) |
| GGA GGG CGG CAT GGC GGG CAC | |
| AG-3′ (Fragment 49) | |
| 5′-GG GGC CTG GAA AGC TGA GAT | (SEQ. ID NO: 57) |
| GGA GGG CGG CAT GGC GGG CAC A-3′ | |
| (Fragment 50) | |
| 5′-GG GGC CTG GAA AGC TGA GAT | (SEQ. ID NO: 58) |
| GGA GGG CGG CAT GGC GGG CAC-3′ | |
| (Fragment 51) | |
| 5′-GG GGC CTG GAA AGC TGA GAT | (SEQ. ID NO: 59) |
| GGA GGG CGG CAT GGC GGG CA-3′ | |
| (Fragment 52) | |
| 5′-GG GGC CTG GAA AGC TGA GAT | (SEQ. ID NO: 60) |
| GGA GGG CGG CAT GGC GGG C-3′ | |
| (Fragment 53) | |
| 5′-GG GGC CTG GAA AGC TGA GAT | (SEQ. ID NO: 61) |
| GGA GGG CGG CAT GGC GGG-3′ | |
| (Fragment 54) | |
| 5′-GC GGC GTG GAA AGC TGA GAT | (SEQ. ID NO: 62) |
| GGA GGG CGG CAT GGC GG-3′ | |
| (Fragment 55) | |
| 5′-GC GGC CTG GAA AGC TGA GAT | (SEQ. ID NO: 63) |
| GGA GGG CGG CAT GGC G-3′ | |
| (Fragment 56) | |
| 5′-GC GGC CTG GAA AGC TGA GAT | (SEQ. ID NO: 64) |
| GGA GGG CGG CAT GGC-3′ | |
| (Fragment 57) | |
| 5′-GC GGC CTG GAA AGC TGA GAT | (SEQ. ID NO: 65) |
| GGA GGG CGG CAT GG-3′ | |
| (Fragment 58) | |
| 5′-GC GGC CTG GAA AGC TGA GAT | (SEQ. ID NO: 66) |
| GGA GGG CGG CAT G-3′ | |
| (Fragment 59) | |
| 5′-GC GGC CTG GAA AGC TGA GAT | (SEQ. ID NO: 67) |
| GGA GGG CGG CAT-3′ (Fragment 60) | |
| 5′-GC GGC CTG GAA AGC TGA GAT | (SEQ. ID NO: 68) |
| GGA GGG CGG CA-3′ (Fragment 61) | |
| 5′-GC GGC CTG GAA AGC TGA GAT | (SEQ. ID NO: 69) |
| GGA GGG CGG C-3′ (Fragment 62) | |
| 5′-GC GGC CTG GAA AGC TGA GAT | (SEQ. ID NO: 70) |
| GGA GGG CGG-3′ (Fragment 63) | |
| 5′-GC GGC CTG GAA AGC TGA GAT | (SEQ. ID NO: 71) |
| GGA GGG CG-3′ (Fragment 64) | |
| 5′-GC GGC CTG GAA AGC TGA GAT | (SEQ. ID NO: 72) |
| GGA GGG C-3′ (Fragment 65) | |
| 5′-GC GGC CTG GAA AGC TGA GAT | (SEQ. ID NO: 73) |
| GGA GGG-3′ (Fragment 66) | |
| 5′-GC GGC CTG GAA AGC TGA GAT | (SEQ. ID NO: 74) |
| GGA GG-3′ (Fragment 67) | |
| 5′-GC GGC CTG GAA AGC TGA GAT | (SEQ. ID NO: 75) |
| GGA G-3′ (Fragment 68) | |
| 5′-GC GGC CTG GAA AGC TGA GAT | (SEQ. ID NO: 76) |
| GGA-3′ (Fragment 69) | |
| 5′-GC GGC CTG GAA AGC TGA GAT | (SEQ. ID NO: 77) |
| GG-3′ (Fragment 70) | |
| 5′-GC GGC CTG GAA AGC TGA GAT | (SEQ. ID NO: 78) |
| G-3′ (Fragment 71) | |
| 5′-GC GGC CTG GAA AGC TGA GAT-3′ | (SEQ. ID NO: 79) |
| (Fragment 72) | |
| 5′-GC GGC CTG GAA AGC TGA GA-3′ | (SEQ. ID NO: 80) |
| (Fragment 73) | |
| 5′-GC GGC CTG GAA AGC TGA G-3′ | (SEQ. ID NO: 81) |
| (Fragment 74) | |
| 5′-GC GGC CTG GAA AGC TGA-3′ | (SEQ. ID NO: 82) |
| (Fragment 75) | |
| 5′-GC GGC CTG GAA AGC TG-3′ | (SEQ. ID NO: 83) |
| (Fragment 76) | |
| 5′-GC GGC CTG GAA AGC T-3′ | (SEQ. ID NO: 84) |
| (Fragment 77) | |
| 5′-GC GGC CTG GAA AGC-3′ | (SEQ. ID NO: 85) |
| (Fragment 78) | |
| 5′-GC GGC CTG GAA AG-3′ | (SEQ. ID NO: 86) |
| (Fragment 79) | |
| 5′-GC GGC CTG GAA A-3′ | (SEQ. ID NO: 87) |
| (Fragment 80) | |
| 5′-GC GGC CTG GAA-3′ | (SEQ. ID NO: 88) |
| (Fragment 81) | |
| 5′-GC GGC CTG GA-3′ | (SEQ. ID NO: 89) |
| (Fragment 82) | |
| 5′-C GGC CTG GAA AGC TGA GAT GGA | (SEQ. ID NO: 90) |
| GGG CGG CAT GGC GGG CAC AGG CTG | |
| GGC-3′ (Fragment 83) | |
| 5′-C GGC CTG GAA AGC TGA GAT GGA | (SEQ. ID NO: 91) |
| GGG CGG CAT GGC GGG CAC AGG CTG | |
| GG-3′ (Fragment 84) | |
| 5′-C GGC CTG GAA AGC TGA GAT GGA | (SEQ. ID NO: 92) |
| GGG CGG CAT GGC GGG CAC AGG CTG | |
| G-3′ (Fragment 85) | |
| 5′-C GGC CTG GAA AGC TGA GAT GGA | (SEQ. ID NO: 93) |
| GGG CGG CAT GGC GGG CAC AGG | |
| CTG-3′ (Fragment 86) | |
| 5′-C GGC CTG GAA AGC TGA GAT GGA | (SEQ. ID NO: 94) |
| GGG CGG CAT GGC GGG CAC AGG | |
| CT-3′ (Fragment 87) | |
| 5′-C GGC CTG GAA AGC TGA GAT GGA | (SEQ. ID NO: 95) |
| GGG CGG CAT GGC GGG CAC AGG C-3′ | |
| (Fragment 88) | |
| 5′-C GGC CTG GAA AGC TGA GAT GGA | (SEQ. ID NO: 96) |
| GGG CGG CAT GGC GGG CAC AGG-3′ | |
| (Fragment 89) | |
| 5′-C GGC CTG GAA AGC TGA GAT GGA | (SEQ. ID NO: 97) |
| GGG CGG CAT GGC GGG CAC AG-3′ | |
| (Fragment 90) | |
| 5′-C GGC CTG GAA AGC TGA GAT GGA | (SEQ. ID NO: 98) |
| GGG CGG CAT GGC GGG CAC A-3′ | |
| (Fragment 91) | |
| 5′-C GGC CTG GAA AGC TGA GAT GGA | (SEQ. ID NO: 99) |
| GGG CGG CAT GGC GGG CAC-3′ | |
| (Fragment 92) | |
| 5′-C GGC CTG GAA AGC TGA GAT GGA | (SEQ. ID NO: 100) |
| GGG CGG CAT GGC GGG CA-3′ | |
| (Fragment 93) | |
| 5′-C GGC CTG GAA AGC TGA GAT GGA | (SEQ. ID NO: 101) |
| GGG CGG CAT GGC GGG C-3′ | |
| (Fragment 94) | |
| 5′-C GGC CTG GAA AGC TGA GAT GGA | (SEQ. ID NO: 102) |
| GGG CGG CAT GGC GGG-3′ | |
| (Fragment 95) | |
| 5′-C GGC CTG GAA AGC TGA GAT GGA | (SEQ. ID NO: 103) |
| GGG CGG CAT GGC GG-3′ | |
| (Fragment 96) | |
| 5′-C GGC CTG GAA AGC TGA GAT GGA | (SEQ. ID NO: 104) |
| GGG CGG CAT GGC G-3′ | |
| (Fragment 97) | |
| 5′-C GGC CTG GAA AGC TGA GAT GGA | (SEQ. ID NO: 105) |
| GGG CGG CAT GGC-3′ (Fragment 98) | |
| 5′-C GGC CTG GAA AGC TGA GAT GGA | (SEQ. ID NO: 106) |
| GGG CGG CAT GG-3′ (Fragment 99) | |
| 5′-C GGC CTG GAA AGC TGA GAT GGA | (SEQ. ID NO: 107) |
| GGG CGG CAT G-3′ (Fragment 100) | |
| 5′-C GGC CTG GAA AGC TGA GAT GGA | (SEQ. ID NO: 108) |
| GGG CGG CAT-3′ (Fragment 101) | |
| 5′-C GGC CTG GAA AGC TGA GAT GGA | (SEQ. ID NO: 109) |
| GGG CGG CA-3′ (Fragment 102) | |
| 5′-C GGC CTG GAA AGC TGA GAT GGA | (SEQ. ID NO: 110) |
| GGG CGG C-3′ (Fragment 103) | |
| 5′-C GGC CTG GAA AGC TGA GAT GGA | (SEQ. ID NO: 111) |
| GGG CGG-3′ (Fragment 104) | |
| 5′-C GGC CTG GAA AGC TGA GAT GGA | (SEQ. ID NO: 112) |
| GGG CG-3′ (Fragment 105) | |
| 5′-C GGC CTG GAA AGC TGA GAT GGA | (SEQ. ID NO: 113) |
| GGG C-3′ (Fragment 106) | |
| 5′-C GGC CTG GAA AGC TGA GAT GGA | (SEQ. ID NO: 114) |
| GGG-3′ (Fragment 107) | |
| 5′-C GGC CTG GAA AGC TGA GAT GGA | (SEQ. ID NO: 115) |
| GG-3′ (Fragment 108) | |
| 5′-C GGC CTG GAA AGC TGA GAT GGA | (SEQ. ID NO: 116) |
| G-3′ (Fragment 109) | |
| 5′-C GGC CTG GAA AGC TGA GAT | (SEQ. ID NO: 117) |
| GGA-3′ (Fragment 110) | |
| 5′-C GGC CTG GAA AGC TGA GAT | (SEQ. ID NO: 118) |
| GG-3′ (Fragment 111) | |
| 5′-C GGC CTG GAA AGC TGA GAT | (SEQ. ID NO: 119) |
| G-3′ (Fragment 112) | |
| 5′-C GGC CTG GAA AGC TGA GAT-3′ | (SEQ. ID NO: 120) |
| (Fragment 113) | |
| 5′-C GGC CTG GAA AGC TGA GA-3′ | (SEQ. ID NO: 121) |
| (Fragment 114) | |
| 5′-C GGC CTG GAA AGC TGA G-3′ | (SEQ. ID NO: 122) |
| (Fragment 115) | |
| 5′-C GGC CTG GAA AGC TGA-3′ | (SEQ. ID NO: 123) |
| (Fragment 116) | |
| 5′-C GGC CTG GAA AGC TG-3′ | (SEQ. ID NO: 124) |
| (Fragment 117) | |
| 5′-C GGC CTG GAA AGC T-3′ | (SEQ. ID NO: 125) |
| (Fragment 118) | |
| 5′-C GGC CTG GAA AGC-3′ | (SEQ. ID NO: 126) |
| (Fragment 119) | |
| 5′-C GGC CTG GAA AG-3′ | (SEQ. ID NO: 127) |
| (Fragment 120) | |
| 5′-C GGC CTG GAA A-3′ | (SEQ. ID NO: 128) |
| (Fragment 121) | |
| 5′-C GGC CTG GAA-3′ | (SEQ. ID NO: 129) |
| (Fragment 122) | |
| 5′-GGC CTG GAA AGC TGA GAT GGA | (SEQ. ID NO: 130) |
| GGG CGG CAT GGC GGG CAC AGG CTG | |
| GGC-3′ (Fragment 123) | |
| 5′-GGC CTG GAA AGC TGA GAT GGA | (SEQ. ID NO: 131) |
| GGG CGG CAT GGC GGG CAC AGG CTG | |
| GG-3′ (Fragment 124) | |
| 5′-GGC CTG GAA AGC TGA GAT GGA | (SEQ. ID NO: 132) |
| GGG CGG CAT GGC GGG CAC AGG CTG | |
| G-3′ (Fragment 125) | |
| 5′-GGC CTG GAA AGC TGA GAT GGA | (SEQ. ID NO: 133) |
| GGG CGG CAT GGC GGG CAC AGG | |
| CTG-3′ (Fragment 126) | |
| 5′-GGC CTG GAA AGC TGA GAT GGA | (SEQ. ID NO: 134) |
| GGG CGG CAT GGC GGG CAC AGG | |
| CT-3′ (Fragment 127) | |
| 5′-GGC CTG GAA AGC TGA GAT GGA | (SEQ. ID NO: 135) |
| GGG CGG CAT GGC GGG CAC AGG C-3′ | |
| (Fragment 128) | |
| 5′-GGC CTG GAA AGC TGA GAT GGA | (SEQ. ID NO: 136) |
| GGG CGG CAT GGC GGG CAC AGG-3′ | |
| (Fragment 129) | |
| 5′-GGC CTG GAA AGC TGA GAT GGA | (SEQ. ID NO: 137) |
| GGG CGG CAT GGC GGG CAC AG-3′ | |
| (Fragment 130) | |
| 5′-GGC CTG GAA AGC TGA GAT GGA | (SEQ. ID NO: 138) |
| GGG CGG CAT GGC GGG CAC A-3′ | |
| (Fragment 131) | |
| 5′-GGC CTG GAA AGC TGA GAT GGA | (SEQ. ID NO: 139) |
| GGG CGG CAT GGC GGG CAC-3′ | |
| (Fragment 132) | |
| 5′-GGC CTG GAA AGC TGA GAT GGA | (SEQ. ID NO: 140) |
| GGG CGG CAT GGC GGG CA-3′ | |
| (Fragment 133) | |
| 5′-GGC CTG GAA AGC TGA GAT GGA | (SEQ. ID NO: 141) |
| GGG CGG CAT GGC GGG C-3′ | |
| (Fragment 134) | |
| 5′-GGC CTG GAA AGC TGA GAT GGA | (SEQ. ID NO: 142) |
| GGG CGG CAT GGC GGG-3′ | |
| (Fragment 135) | |
| 5′-GGC CTG GAA AGC TGA GAT GGA | (SEQ. ID NO: 143) |
| GGG CGG CAT GGC GG-3′ | |
| (Fragment 136) | |
| 5′-GGC CTG GAA AGC TGA GAT GGA | (SEQ. ID NO: 144) |
| GGG CGG CAT GGC G-3′ | |
| (Fragment 137) | |
| 5′-GGC CTG GAA AGC TGA GAT GGA | (SEQ. ID NO: 145) |
| GGG CGG CAT GGC-3′ | |
| (Fragment 138) | |
| 5′-GGC CTG GAA AGC TGA GAT GGA | (SEQ. ID NO: 146) |
| GGG CGG CAT GG-3′ (Fragment 139) | |
| 5′-GGC CTG GAA AGC TGA GAT GGA | (SEQ. ID NO: 147) |
| GGG CGG CAT G-3′ (Fragment 140) | |
| 5′-GGC CTG GAA AGC TGA GAT GGA | (SEQ. ID NO: 148) |
| GGG CGG CAT-3′ (Fragment 141) | |
| 5′-GGC CTG GAA AGC TGA GAT GGA | (SEQ. ID NO: 149) |
| GGG CGG CA-3′ (Fragment 142) | |
| 5′-GGC CTG GAA AGC TGA GAT GGA | (SEQ. ID NO: 150) |
| GGG CGG C-3′ (Fragment 143) | |
| 5′-GGC CTG GAA AGC TGA GAT GGA | (SEQ. ID NO: 151) |
| GGG CGG-3′ (Fragment 144) | |
| 5′-GGC CTG GAA AGC TGA GAT GGA | (SEQ. ID NO: 152) |
| GGG CG-3′ (Fragment 145) | |
| 5′-GGC CTG GAA AGC TGA GAT GGA | (SEQ. ID NO: 153) |
| GGG C-3′ (Fragment 146) | |
| 5′-GGC CTG GAA AGC TGA GAT GGA | (SEQ. ID NO: 154) |
| GGG-3′ (Fragment 147) | |
| 5′-GGC CTG GAA AGC TGA GAT GGA | (SEQ. ID NO: 155) |
| GG-3′ (Fragment 148) | |
| 5′-GGC CTG GAA AGC TGA GAT GGA | (SEQ. ID NO: 156) |
| G-3′ (Fragment 149) | |
| 5′-GGC CTG GAA AGC TGA GAT | (SEQ. ID NO: 157) |
| GGA-3′ (Fragment 150) | |
| 5′-GGC CTG GAA AGC TGA GAT GG-3′ | (SEQ. ID NO: 158) |
| (Fragment 151) | |
| 5′-GGC CTG GAA AGC TGA GAT G-3′ | (SEQ. ID NO: 159) |
| (Fragment 152) | |
| 5′-GGC CTG GAA AGC TGA GAT-3′ | (SEQ. ID NO: 160) |
| (Fragment 153) | |
| 5′-GGC CTG GAA AGC TGA GA-3′ | (SEQ. ID NO: 161) |
| (Fragment 154) | |
| 5′-GGC CTG GAA AGC TGA G-3′ | (SEQ. ID NO: 162) |
| (Fragment 155) | |
| 5′-GGC CTG GAA AGC TGA-3′ | (SEQ. ID NO: 163) |
| (Fragment 156) | |
| 5′-GGC CTG GAA AGC TG-3′ | (SEQ. ID NO: 164) |
| (Fragment 157) | |
| 5′-GGC CTG GAA AGC T-3′ | (SEQ. ID NO: 165) |
| (Fragment 158) | |
| 5′-GGC CTG GAA AGC-3′ | (SEQ. ID NO: 166) |
| (Fragment 159) | |
| 5′-GGC CTG GAA AG-3′ | (SEQ. ID NO: 167) |
| (Fragment 160) | |
| 5′-GGC CTG GAA A-3′ | (SEQ. ID NO: 168) |
| (Fragment 161) | |
| 5′-GC CTG GAA AGC TGA GAT GGA | (SEQ. ID NO: 169) |
| GGG CGG CAT GGC GGG CAC AGG CTG | |
| GGC-3′ (Fragment 162) | |
| 5′-GC CTG GAA AGC TGA GAT GGA | (SEQ. ID NO: 170) |
| GGG CGG CAT GGC GGG CAC AGG CTG | |
| GG-3′ (Fragment 163) | |
| 5′-GC CTG GAA AGC TGA GAT GGA | (SEQ. ID NO: 171) |
| GGG CGG CAT GGC GGG CAC AGG CTG | |
| G-3′ (Fragment 164) | |
| 5′-GC CTG GAA AGC TGA GAT GGA | (SEQ. ID NO: 172) |
| GGG CGG CAT GGC GGG CAC AGG | |
| CTG-3′ (Fragment 165) | |
| 5′-GC CTG GAA AGC TGA GAT GGA | (SEQ. ID NO: 173) |
| GGG CGG CAT GGC GGG CAC AGG | |
| CT-3′ (Fragment 166) | |
| 5′-GC CTG GAA AGC TGA GAT GGA | (SEQ. ID NO: 174) |
| GGG CGG CAT GGC GGG CAC AGG C-3′ | |
| (Fragment 167) | |
| 5′-GC CTG GAA AGC TGA GAT GGA | (SEQ. ID NO: 175) |
| GGG CGG CAT GGC GGG CAC AGG-3′ | |
| (Fragment 168) | |
| 5′-GC CTG GAA AGC TGA GAT GGA | (SEQ. ID NO: 176) |
| GGG CGG CAT GGC GGG CAC AG-3′ | |
| (Fragment 169) | |
| 5′-GC CTG GAA AGC TGA GAT GGA | (SEQ. ID NO: 177) |
| GGG CGG CAT GGC GGG CAC A-3′ | |
| (Fragment 170) | |
| 5′-GC CTG GAA AGC TGA GAT GGA | (SEQ. ID NO: 178) |
| GGG CGG CAT GGC GGG CAC-3′ | |
| (Fragment 171) | |
| 5′-GC CTG GAA AGC TGA GAT GGA | (SEQ. ID NO: 179) |
| GGG CGG CAT GGC GGG CA-3′ | |
| (Fragment 172) | |
| 5′-GC CTG GAA AGC TGA GAT GGA | (SEQ. ID NO: 180) |
| GGG CGG CAT GGC GGG C-3′ | |
| (Fragment 173) | |
| 5′-GC CTG GAA AGC TGA GAT GGA | (SEQ. ID NO: 181) |
| GGG CGG CAT GGC GGG-3′ | |
| (Fragment 174) | |
| 5′-GC CTG GAA AGC TGA GAT GGA | (SEQ. ID NO: 182) |
| GGG CGG CAT GGC GG-3′ | |
| (Fragment 175) | |
| 5′-GC CTG GAA AGC TGA GAT GGA | (SEQ. ID NO: 183) |
| GGG CGG CAT GGC G-3′ | |
| (Fragment 176) | |
| 5′-GC CTG GAA AGC TGA GAT GGA | (SEQ. ID NO: 184) |
| GGG CGG CAT GGC-3′ | |
| (Fragment 177) | |
| 5′-GC CTG GAA AGC TGA GAT GGA | (SEQ. ID NO: 185) |
| GGG CGG CAT GG-3′ (Fragment 178) | |
| 5′-GC CTG GAA AGC TGA GAT GGA | (SEQ. ID NO: 186) |
| GGG CGG CAT G-3′ (Fragment 179) | |
| 5′-GC CTG GAA AGC TGA GAT GGA | (SEQ. ID NO: 187) |
| GGG CGG CAT-3′ (Fragment 180) | |
| 5′-GC CTG GAA AGC TGA GAT GGA | (SEQ. ID NO: 188) |
| GGG CGG CA-3′ (Fragment 181) | |
| 5′-GC CTG GAA AGC TGA GAT GGA | (SEQ. ID NO: 189) |
| GGG CGG C-3′ (Fragment 182) | |
| 5′-GC CTG GAA AGC TGA GAT GGA | (SEQ. ID NO: 190) |
| GGG CGG-3′ (Fragment 183) | |
| 5′-GC CTG GAA AGC TGA GAT GGA | (SEQ. ID NO: 191) |
| GGG CG-3′ (Fragment 184) | |
| 5′-GC CTG GAA AGC TGA GAT GGA | (SEQ. ID NO: 192) |
| GGG C-3′ (Fragment 185) | |
| 5′-GC CTG GAA AGC TGA GAT GGA | (SEQ. ID NO: 193) |
| GGG-3′ (Fragment 186) | |
| 5′-GC CTG GAA AGC TGA GAT GGA | (SEQ. ID NO: 194) |
| GG-3′ (Fragment 187) | |
| 5′-GC CTG GAA AUC TGA GAT GGA | (SEQ. ID NO: 195) |
| G-3′ (Fragment 188) | |
| 5′-GC CTG GAA AGC TGA GAT GGA-3′ | (SEQ. ID NO: 196) |
| (Fragment 189) | |
| 5′-GC CTG GAA AGC TGA GAT GG-3′ | (SEQ. ID NO: 197) |
| (Fragment 190) | |
| 5′-GC CTG GAA AGC TGA GAT G-3′ | (SEQ. ID NO: 198) |
| (Fragment 191) | |
| 5′-GC CTG GAA AGC TGA GAT-3′ | (SEQ. ID NO: 199) |
| (Fragment 192) | |
| 5′-GC CTG GAA AGC TGA GA-3′ | (SEQ. ID NO: 200) |
| (Fragment 193) | |
| 5′-GC CTG GAA AGC TGA G-3′ | (SEQ. ID NO: 201) |
| (Fragment 194) | |
| 5′-GC CTG GAA AGC TGA-3′ | (SEQ. ID NO: 202) |
| (Fragment 195) | |
| 5′-GC CTG GAA AGC TG-3′ | (SEQ. ID NO: 203) |
| (Fragment 196) | |
| 5′-GC CTG GAA AGC T-3′ | (SEQ. ID NO: 204) |
| (Fragment 197) | |
| 5′-GC CTG GAA AGC-3′ | (SEQ. ID NO: 205) |
| (Fragment 198) | |
| 5′-GC CTG GAA AG-3′ | (SEQ. ID NO: 206) |
| (Fragment 199) | |
| 5′-C CTG GAA AGC TGA GAT GGA GGG | (SEQ. ID NO: 207) |
| CGG CAT GGC GGG CAC AGG CTG | |
| GGC-3′ (Fragment 200) | |
| 5′-C CTG GAA AGC TGA GAT GGA GGG | (SEQ. ID NO: 208) |
| CGG CAT GGC GGG CAC AGG CTG | |
| GG-3′ (Fragment 201) | |
| 5′-C CTG GAA AGC TGA GAT GGA GGG | (SEQ. ID NO: 209) |
| CGG CAT GGC GGG CAC AGG CTG G-3′ | |
| (Fragment 202) | |
| 5′-C CTG GAA AGC TGA GAT GGA GGG | (SEQ. ID NO: 210) |
| CGG CAT GGC GGG CAC AGG CTG-3′ | |
| (Fragment 203) | |
| 5′-C CTG GAA AGC TGA GAT GGA GGG | (SEQ. ID NO: 211) |
| CGG CAT GGC GGG CAC AGG CT-3′ | |
| (Fragment 204) | |
| 5′-C CTG GAA AGC TGA GAT GGA GGG | (SEQ. ID NO: 212) |
| CGG CAT GGC GGG CAC AGG C-3′ | |
| (Fragment 205) | |
| 5′-C CTG GAA AGC TGA GAT GGA GGG | (SEQ. ID NO: 213) |
| CGG CAT GGC GGG CAC AGG-3′ | |
| (Fragment 206) | |
| 5′-C CTG GAA AGC TGA GAT GGA GGG | (SEQ. ID NO: 214) |
| CGG CAT GGC GGG CAC AG-3′ | |
| (Fragment 207) | |
| 5′-C CTG GAA AGC TGA GAT GGA GGG | (SEQ. ID NO: 215) |
| CGG CAT GGC GGG CAC A-3′ | |
| (Fragment 208) | |
| 5′-C CTG GAA AGC TGA GAT GGA GGG | (SEQ. ID NO: 216) |
| CGG CAT GGC GGG CAC-3′ | |
| (Fragment 209) | |
| 5′-C CTG GAA AGC TGA GAT GGA GGG | (SEQ. ID NO: 217) |
| CGG CAT GGC GGG CA-3′ | |
| (Fragment 210) | |
| 5′-C CTG GAA AGC TGA GAT GGA GGG | (SEQ. ID NO: 218) |
| CGG CAT GGC GGG C-3′ | |
| (Fragment 211) | |
| 5′-C CTG GAA AGC TGA GAT GGA GGG | (SEQ. ID NO: 219) |
| CGG CAT GGC GGG-3′ | |
| (Fragment 212) | |
| 5′-C CTG GAA AGC TGA GAT GGA GGG | (SEQ. ID NO: 220) |
| CGG CAT GGC GG-3′ (Fragment 213) | |
| 5′-C CTG GAA AGC TGA GAT GGA GGG | (SEQ. ID NO: 221) |
| CGG CAT GGC G-3′ (Fragment 214) | |
| 5′-C CTG GAA AGC TGA GAT GGA GGG | (SEQ. ID NO: 222) |
| CGG CAT GGC-3′ (Fragment 215) | |
| 5′-C CTG GAA AGC TGA GAT GGA GGG | (SEQ. ID NO: 223) |
| CGG CAT GG-3′ (Fragment 216) | |
| 5′-C CTG GAA AGC TGA GAT GGA GGG | (SEQ. ID NO: 224) |
| CGG CAT G-3′ (Fragment 217) | |
| 5′-C CTG GAA AGC TGA GAT GGA GGG | (SEQ. ID NO: 225) |
| CGG CAT-3′ (Fragment 218) | |
| 5′-C CTG GAA AGC TGA GAT GGA GGG | (SEQ. ID NO: 226) |
| CGG CA-3′ (Fragment 219) | |
| 5′-C CTG GAA AGC TGA GAT GGA GGG | (SEQ. ID NO: 227) |
| CGG C-3′ (Fragment 220) | |
| 5′-C CTG GAA AGC TGA GAT GGA GGG | (SEQ. ID NO: 228) |
| CGG-3′ (Fragment 221) | |
| 5′-C CTG GAA AGC TGA GAT GGA GGG | (SEQ. ID NO: 229) |
| CG-3′ (Fragment 222) | |
| 5′-C CTG GAA AGC TGA GAT GGA GGG | (SEQ. ID NO: 230) |
| C-3′ (Fragment 223) | |
| 5′-C CTU GAA AGC TGA GAT GGA | (SEQ. ID NO: 231) |
| GGG-3′ (Fragment 224) | |
| 5′-C CTG GAA AGC TGA GAT GGA | (SEQ. ID NO: 232) |
| GG-3′ (Fragment 225) | |
| 5′-C CTG GAA AGC TGA GAT GGA | (SEQ. ID NO: 233) |
| G-3′ (Fragment 226) | |
| 5′-C CTG GAA AGC TGA GAT GGA-3′ | (SEQ. ID NO: 234) |
| (Fragment 227) | |
| 5′-C CTG GAA AGC TGA GAT GG-3′ | (SEQ. ID NO: 235) |
| (Fragment 228) | |
| 5′-C CTG GAA AGC TGA GAT G-3′ | (SEQ. ID NO: 236) |
| (Fragment 229) | |
| 5′-C CTG GAA AGC TGA GAT-3′ | (SEQ. ID NO: 237) |
| (Fragment 230) | |
| 5′-C CTG GAA AGC TGA GA-3′ | (SEQ. ID NO: 238) |
| (Fragment 231) | |
| 5′-C CTG GAA AGC TGA G-3′ | (SEQ. ID NO: 239) |
| (Fragment 232) | |
| 5′-C CTG GAA AGC TGA-3′ | (SEQ. ID NO: 240) |
| (Fragment 233) | |
| 5′-C CTG GAA AGC TG-3′ | (SEQ. ID NO: 241) |
| (Fragment 234) | |
| 5′-C CTG GAA AGC T-3′ | (SEQ. ID NO: 242) |
| (Fragment 235) | |
| 5′-C CTG GAA AGC-3′ | (SEQ. ID NO: 243) |
| (Fragment 236) | |
| 5′-CTG GAA AGC TGA GAT GGA GGG | (SEQ. ID NO: 244) |
| CGG CAT GGC GGG CAC AGG CTG | |
| GGC-3′ (Fragment 237) | |
| 5′-CTG GAA AGC TGA GAT GGA GGG | (SEQ. ID NO: 245) |
| CGG CAT GGC GGG CAC AGG CTG | |
| GG-3′ (Fragment 238) | |
| 5′-CTG GAA AGC TGA GAT GGA GGG | (SEQ. ID NO: 246) |
| CGG CAT GGC GGG CAC AGG CTG G-3′ | |
| (Fragment 239) | |
| 5′-CTG GAA AGC TGA GAT GGA GGG | (SEQ. ID NO: 247) |
| CGG CAT GGC GGG CAC AGG CTG-3′ | |
| (Fragment 240) | |
| 5′-CTG GAA AGC TGA GAT GGA GGG | (SEQ. ID NO: 248) |
| CGG CAT GGC GGG CAC AGG CT-3′ | |
| (Fragment 241) | |
| 5′-CTG GAA AGC TGA GAT GGA GGG | (SEQ. ID NO: 249) |
| CGG CAT GGC GGG CAC AGG C-3′ | |
| (Fragment 242) | |
| 5′-CTG GAA AGC TGA GAT GGA GGG | (SEQ. ID NO: 250) |
| CGG CAT GGC GGG CAC AGG-3′ | |
| (Fragment 243) | |
| 5′-CTG GAA AGC TGA GAT GGA GGG | (SEQ. ID NO: 251) |
| CGG CAT GGC GGG CAC AG-3′ | |
| (Fragment 244) | |
| 5′-CTG GAA AGC TGA GAT GGA GGG | (SEQ. ID NO: 252) |
| CGG CAT GGC GGG CAC A-3′ | |
| (Fragment 245) | |
| 5′-CTG GAA AGC TGA GAT GGA GGG | (SEQ. ID NO: 253) |
| CGG CAT GGC GGG CAC-3′ | |
| (Fragment 246) | |
| 5′-CTG GAA AGC TGA GAT GGA GGG | (SEQ. ID NO: 254) |
| CGG CAT GGC GGG CA-3′ | |
| (Fragment 247) | |
| 5′-CTG GAA AGC TGA GAT GGA GGG | (SEQ. ID NO: 255) |
| CGG CAT GGC GGG C-3′ | |
| (Fragment 248) | |
| 5′-CTG GAA AGC TGA GAT GGA GGG | (SEQ. ID NO: 256) |
| CGG CAT GGC GGG-3′ | |
| (Fragment 249) | |
| 5′-CTG GAA AGC TGA GAT GGA GGG | (SEQ. ID NO: 257) |
| CGG CAT GGC GG-3′ (Fragment 250) | |
| 5′-CTG GAA AGC TGA GAT GGA GGG | (SEQ. ID NO: 258) |
| CGG CAT GGC G-3′ (Fragment 251) | |
| 5′-CTG GAA AGC TGA GAT GGA GGG | (SEQ. ID NO: 259) |
| CGG CAT GGC-3′ (Fragment 252) | |
| 5′-CTG GAA AGC TGA GAT GGA GGG | (SEQ. ID NO: 260) |
| CGG CAT GG-3′ (Fragment 253) | |
| 5′-CTG GAA AGC TGA GAT GGA GGG | (SEQ. ID NO: 261) |
| CGG CAT G-3′ (Fragment 254) | |
| 5′-CTG GAA AGC TGA GAT GGA GGG | (SEQ. ID NO: 262) |
| CGG CAT-3′ (Fragment 255) | |
| 5′-CTG GAA AGC TGA GAT GGA GGG | (SEQ. ID NO: 263) |
| CGG CA-3′ (Fragment 256) | |
| 5′-CTG GAA AGC TGA GAT GGA GGG | (SEQ. ID NO: 264) |
| CGG C-3′ (Fragment 257) | |
| 5′-CTG GAA AGC TGA GAT GGA GGG | (SEQ. ID NO: 265) |
| CGG-3′ (Fragment 258) | |
| 5′-CTG GAA AGC TGA GAT GGA GGG | (SEQ. ID NO: 266) |
| CG-3′ (Fragment 259) | |
| 5′-CTG GAA AGC TGA GAT GGA GGG | (SEQ. ID NO: 267) |
| C-3′ (Fragment 260) | |
| 5′-CTG GAA AGC TGA GAT GGA | (SEQ. ID NO: 268) |
| GGG-3′ (Fragment 261) | |
| 5′-CTG GAA AGC TGA GAT GGA GG-3′ | (SEQ. ID NO: 269) |
| (Fragment 262) | |
| 5′-CTG GAA AGC TGA GAT GGA G-3′ | (SEQ. ID NO: 270) |
| (Fragment 263) | |
| 5′-CTG GAA AGC TGA GAT GGA-3′ | (SEQ. ID NO: 271) |
| (Fragment 264) | |
| 5′-CTG GAA AGC TGA GAT GG-3′ | (SEQ. ID NO: 272) |
| (Fragment 265) | |
| 5′-CTG GAA AGC TGA GAT G-3′ | (SEQ. ID NO: 273) |
| (Fragment 266) | |
| 5′-CTG GAA AGC TGA GAT-3′ | (SEQ. ID NO: 274) |
| (Fragment 267) | |
| 5′-CTG GAA AGC TGA GA-3′ | (SEQ. ID NO: 275) |
| (Fragment 268) | |
| 5′-CTG GAA AGC TGA G-3′ | (SEQ. ID NO: 276) |
| (Fragment 269) | |
| 5′-CTG GAA AGC TGA-3′ | (SEQ. ID NO: 277) |
| (Fragment 270) | |
| 5′-CTG GAA AGC TG-3′ | (SEQ. ID NO: 278) |
| (Fragment 271) | |
| 5′-CTG GAA AGC T-3′ | (SEQ. ID NO: 279) |
| (Fragment 272) | |
| 5′-TG GAA AGC TGA GAT GGA GGG | (SEQ. ID NO: 280) |
| CGG CAT GGC GGG CAC AGG CTG | |
| GGC-3′ (Fragment 273) | |
| 5′-TG GAA AGC TGA GAT GGA GGG | (SEQ. ID NO: 281) |
| CGG CAT GGC GGG CAC AGG CTG | |
| GG-3′ (Fragment 274) | |
| 5′-TG GAA AGC TGA GAT GGA GGG | (SEQ. ID NO: 282) |
| CGG CAT GGC GGG CAC AGG CTG G-3′ | |
| (Fragment 275) | |
| 5′-TG GAA AGC TGA GAT GGA GGG | (SEQ. ID NO: 283) |
| CGG CAT GGC GGG CAC AGG CTG-3′ | |
| (Fragment 276) | |
| 5′-TG GAA AGC TGA GAT GGA GGG | (SEQ. ID NO: 284) |
| CGG CAT GGC GGG CAC AGG CT-3′ | |
| (Fragment 277) | |
| 5′-TG GAA AGC TGA GAT GGA GGG | (SEQ. ID NO: 285) |
| CGG CAT GGC GGG CAC AGG C-3′ | |
| (Fragment 278) | |
| 5′-TG GAA AGC TGA GAT GGA GGG | (SEQ. ID NO: 286) |
| CGG CAT GGC GGG CAC AGG-3′ | |
| (Fragment 279) | |
| 5′-TG GAA AGC TGA GAT GGA GGG | (SEQ. ID NO: 287) |
| CGG CAT GGC GGG CAC AG-3′ | |
| (Fragment 280) | |
| 5′-TG GAA AGC TGA GAT GGA GGG | (SEQ. ID NO: 288) |
| CGG CAT GGC GGG CAC A-3′ | |
| (Fragment 281) | |
| 5′-TG GAA AGC TGA GAT GGA GGG | (SEQ. ID NO: 289) |
| CGG CAT GGC GGG CAC-3′ | |
| (Fragment 282) | |
| 5′-TG GAA AGC TGA GAT GGA GGG | (SEQ. ID NO: 290) |
| CGG CAT GGC GGG CA-3′ | |
| (Fragment 283) | |
| 5′-TG GAA AGC TGA GAT GGA GGG | (SEQ. ID NO: 291) |
| CGG CAT GGC GGG C-3′ | |
| (Fragment 284) | |
| 5′-TG GAA AGC TGA GAT GGA GGG | (SEQ. ID NO: 292) |
| CGG CAT GGC GGG-3′ | |
| (Fragment 285) | |
| 5′-TG GAA AGC TGA GAT GGA GGG | (SEQ. ID NO: 293) |
| CGG CAT GGC GG-3′ (Fragment 286) | |
| 5′-TG GAA AGC TGA GAT GGA GGG | (SEQ. ID NO: 294) |
| CGG CAT GGC G-3′ (Fragment 287) | |
| 5′-TG GAA AGC TGA GAT GGA GGG | (SEQ. ID NO: 295) |
| CGG CAT GGC-3′ (Fragment 288) | |
| 5′-TG GAA AGC TGA GAT GGA GGG | (SEQ. ID NO: 296) |
| CGG CAT GG-3′ (Fragment 289) | |
| 5′-TG GAA AGC TGA GAT GGA GGG | (SEQ. ID NO: 297) |
| CGG CAT G-3′ (Fragment 290) | |
| 5′-TG GAA AGC TGA GAT GGA GGG | (SEQ. ID NO: 298) |
| CGG CAT-3′ (Fragment 291) | |
| 5′-TG GAA AGC TGA GAT GGA GGG | (SEQ. ID NO: 299) |
| CGG CA-3′ (Fragment 292) | |
| 5′-TG GAA AGC TGA GAT GGA GGG | (SEQ. ID NO: 300) |
| CGG C-3′ (Fragment 293) | |
| 5′-TG GAA AGC TGA GAT GGA GGG | (SEQ. ID NO: 301) |
| CGG-3′ (Fragment 294) | |
| 5′-TG GAA AGC TGA GAT GGA GGG | (SEQ. ID NO: 302) |
| CG-3′ (Fragment 295) | |
| 5′-TG GAA AGC TGA GAT GGA GGG | (SEQ. ID NO: 303) |
| C-3′ (Fragment 296) | |
| 5′-TG GAA AGC TGA GAT GGA GGG-3′ | (SEQ. ID NO: 304) |
| (Fragment 297) | |
| 5′-TG GAA AGC TGA GAT GGA GG-3′ | (SEQ. ID NO: 305) |
| (Fragment 298) | |
| 5′-TG GAA AGC TGA GAT GGA G-3′ | (SEQ. ID NO: 306) |
| (Fragment 299) | |
| 5′-TG GAA AGC TGA GAT GGA-3′ | (SEQ. ID NO: 307) |
| (Fragment 300) | |
| 5′-TG GAA AGC TGA GAT GG-3′ | (SEQ. ID NO: 308) |
| (Fragment 301) | |
| 5′-TG GAA AGC TGA GAT G-3′ | (SEQ. ID NO: 309) |
| (Fragment 302) | |
| 5′-TG GAA AGC TGA GAT-3′ | (SEQ. ID NO: 310) |
| (Fragment 303) | |
| 5′-TG GAA AGC TGA GA-3′ | (SEQ. ID NO: 311) |
| (Fragment 304) | |
| 5′-TG GAA AGC TGA G-3′ | (SEQ. ID NO: 312) |
| (Fragment 305) | |
| 5′-TG GAA AGC TGA-3′ | (SEQ. ID NO: 313) |
| (Fragment 306) | |
| 5′-TG GAA AGC TG-3′ | (SEQ. ID NO: 314) |
| (Fragment 307) | |
| 5′-G GAA AGC TGA GAT GGA GGG CGG | (SEQ. ID NO: 315) |
| CAT GGC GGG CAC AGG CTG GGC-3′ | |
| (Fragment 308) | |
| 5′-G GAA AGC TGA GAT GGA GGG CGG | (SEQ. ID NO: 316) |
| CAT GGC GGG CAC AGG CTG GG-3′ | |
| (Fragment 309) | |
| 5′-G GAA AGC TGA GAT GGA GGG CGG | (SEQ. ID NO: 317) |
| CAT GGC GGG CAC AGG CTG G-3′ | |
| (Fragment 310) | |
| 5′-G GAA AGC TGA GAT GGA GGG CGG | (SEQ. ID NO: 318) |
| CAT GGC GGG CAC AGG CTG-3′ | |
| (Fragment 311) | |
| 5′-G GAA AGC TGA GAT GGA GGG CGG | (SEQ. ID NO: 319) |
| CAT GGC GGG CAC AGG CT-3′ | |
| (Fragment 312) | |
| 5′-G GAA AGC TGA GAT GGA GGG CGG | (SEQ. ID NO: 320) |
| CAT GGC GGG CAC AGG C-3′ | |
| (Fragment 313) | |
| 5′-G GAA AGC TGA GAT GGA GGG CGG | (SEQ. ID NO: 321) |
| CAT GGC GGG CAC AGG-3′ | |
| (Fragment 314) | |
| 5′-G GAA AGC TGA GAT GGA GGG CGG | (SEQ. ID NO: 322) |
| CAT GGC GGG CAC AG-3′ | |
| (Fragment 315) | |
| 5′-G GAA AGC TGA GAT GGA GGG CGG | (SEQ. ID NO: 323) |
| CAT GGC GGG CAC A-3′ | |
| (Fragment 316) | |
| 5′-G GAA AGC TGA GAT GGA GGG CGG | (SEQ. ID NO: 324) |
| CAT GGC GGG CAC-3′ | |
| (Fragment 317) | |
| 5′-G GAA AGC TGA GAT GGA GGG CGG | (SEQ. ID NO: 325) |
| CAT GGC GGG CA-3′ (Fragment 318) | |
| 5′-G GAA AGC TGA GAT GGA GGG CGG | (SEQ. ID NO: 326) |
| CAT GGC GGG C-3′ (Fragment 319) | |
| 5′-G GAA AGC TGA GAT GGA GGG CGG | (SEQ. ID NO: 327) |
| CAT GGC GGG-3′ (Fragment 320) | |
| 5′-G GAA AGC TGA GAT GGA GGG CGG | (SEQ. ID NO: 328) |
| CAT GGC GG-3′ (Fragment 321) | |
| 5′-G GAA AGC TGA GAT GGA GGG CGG | (SEQ. ID NO: 329) |
| CAT GGC G-3′ (Fragment 322) | |
| 5′-G GAA AGC TGA GAT GGA GGG CGG | (SEQ. ID NO: 330) |
| CAT GGC-3′ (Fragment 323) | |
| 5′-G GAA AGC TGA GAT GGA GGG CGG | (SEQ. ID NO: 331) |
| CAT GG-3′ (Fragment 324) | |
| 5′-G GAA AGC TGA GAT GGA GGG CGG | (SEQ. ID NO: 332) |
| CAT G-3′ (Fragment 325) | |
| 5′-G GAA AGC TGA GAT GGA GGG CGG | (SEQ. ID NO: 333) |
| CAT-3′ (Fragment 326) | |
| 5′-G GAA AGC TGA GAT GGA GGG CGG | (SEQ. ID NO: 334) |
| CA-3′ (Fragment 327) | |
| 5′-G GAA AGC TGA GAT GGA GGG CGG | (SEQ. ID NO: 335) |
| C-3′ (Fragment 328) | |
| 5′-G GAA AGC TGA GAT GGA GGG | (SEQ. ID NO: 336) |
| CGG-3′ (Fragment 329) | |
| 5′-G GAA AGC TGA GAT GGA GGG | (SEQ. ID NO: 337) |
| CG-3′ (Fragment 330) | |
| 5′-G GAA AGC TGA GAT GGA GGG | (SEQ. ID NO: 338) |
| C-3′ (Fragment 331) | |
| 5′-G GAA AGC TGA GAT GGA GGG-3′ | (SEQ. ID NO: 339) |
| (Fragment 332) | |
| 5′-G GAA AGC TGA GAT GGA GG-3′ | (SEQ. ID NO: 340) |
| (Fragment 333) | |
| 5′-G GAA AGC TGA GAT GGA G-3′ | (SEQ. ID NO: 341) |
| (Fragment 334) | |
| 5′-G GAA AGC TGA GAT GGA-3′ | (SEQ. ID NO: 342) |
| (Fragment 335) | |
| 5′-G GAA AGC TGA GAT GG-3′ | (SEQ. ID NO: 343) |
| (Fragment 336) | |
| 5′-G GAA AGC TGA GAT G-3′ | (SEQ. ID NO: 344) |
| (Fragment 337) | |
| 5′-G GAA AGC TGA GAT-3′ | (SEQ. ID NO: 345) |
| (Fragment 338) | |
| 5′-G GAA AGC TGA GA-3′ | (SEQ. ID NO: 346) |
| (Fragment 339) | |
| 5′-G GAA AGC TGA G-3′ | (SEQ. ID NO: 347) |
| (Fragment 340) | |
| 5′-G GAA AGC TGA-3′ | (SEQ. ID NO: 348) |
| (Fragment 341) | |
| 5′-GAA AGC TGA GAT GGA GGG CGG | (SEQ. ID NO: 349) |
| CAT GGC GGG CAC AGG CTG GGC-3′ | |
| (Fragment 342) | |
| 5′-GAA AGC TGA GAT GGA GGG CGG | (SEQ. ID NO: 350) |
| CAT GGC GGG CAC AGG CTG GG-3′ | |
| (Fragment 343) | |
| 5′-GAA AGC TGA GAT GGA GGG CGG | (SEQ. ID NO: 351) |
| CAT GGC GGG CAC AGG CTG G-3′ | |
| (Fragment 344) | |
| 5′-GAA AGC TGA GAT GGA GGG CGG | (SEQ. ID NO: 352) |
| CAT GGC GGG CAC AGG CTG-3′ | |
| (Fragment 345) | |
| 5′-GAA AGC TGA GAT GGA GGG CGG | (SEQ. ID NO: 353) |
| CAT GGC GGG CAC AGG CT-3′ | |
| (Fragment 346) | |
| 5′-GAA AGC TGA GAT GGA GGG CGG | (SEQ. ID NO: 354) |
| CAT GGC GGG CAC AGG C-3′ | |
| (Fragment 347) | |
| 5′-GAA AGC TGA GAT GGA GGG CGG | (SEQ. ID NO: 355) |
| CAT GGC GGG CAC AGG-3′ | |
| (Fragment 348) | |
| 5′-GAA AGC TGA GAT GGA GGG CGG | (SEQ. ID NO: 356) |
| CAT GGC GGG CAC AG-3′ | |
| (Fragment 349) | |
| 5′-GAA AGC TGA GAT GGA GGG CGG | (SEQ. ID NO: 357) |
| CAT GGG GGG CAG A-3′ | |
| (Fragment 350) | |
| 5′-GAA AGC TGA GAT GGA GGG CGG | (SEQ. ID NO: 358) |
| CAT GGC GGG GAG-3′ | |
| (Fragment 351) | |
| 5′-GAA AGG TGA GAT GGA GGG CGG | (SEQ. ID NO: 359) |
| CAT GGC GGG CA-3′ (Fragment 352) | |
| 5′-GAA AGC TGA GAT GGA GGG CGG | (SEQ. ID NO: 360) |
| CAT GGC GGG C-3′ (Fragment 353) | |
| 5′-GAA AGC TGA GAT GGA GGG CGG | (SEQ. ID NO: 361) |
| CAT GGC GGG-3′ (Fragment 354) | |
| 5′-GAA AGC TGA GAT GGA GGG CGG | (SEQ. ID NO: 362) |
| CAT GGC GG-3′ (Fragment 355) | |
| 5′-GAA AGC TGA GAT GGA GGG CGG | (SEQ. ID NO: 363) |
| CAT GGC G-3′ (Fragment 356) | |
| 5′-GAA AGC TGA GAT GGA GGG CGG | (SEQ. ID NO: 364) |
| CAT GGC-3′ (Fragment 357) | |
| 5′-GAA AGC TGA GAT GGA GGG CGG | (SEQ. ID NO: 365) |
| CAT GG-3′ (Fragment 358) | |
| 5′-GAA AGG TGA GAT GGA GGG GGG | (SEQ. ID NO: 366) |
| CAT G-3′ (Fragment 359) | |
| 5′-GAA AGC TGA GAT GGA GGG CGG | (SEQ. ID NO: 367) |
| CAT-3′ (Fragment 360) | |
| 5′-GAA AGC TGA GAT GGA GGG CGG | (SEQ. ID NO: 368) |
| GA-3′ (Fragment 361) | |
| 5′-GAA AGC TGA GAT GGA GGG CGG | (SEQ. ID NO: 369) |
| G-3′ (Fragment 362) | |
| 5′-GAA AGC TGA GAT GGA GGG | (SEQ. ID NO: 370) |
| CGG-3′ (Fragment 363) | |
| 5′-GAA AGC TGA GAT GGA GGG CG-3′ | (SEQ. ID NO: 371) |
| (Fragment 364) | |
| 5′-GAA AGC TGA GAT GGA GGG C-3′ | (SEQ. ID NO: 372) |
| (Fragment 365) | |
| 5′-GAA AGC TGA GAT GGA GGG-3′ | (SEQ. ID NO: 373) |
| (Fragment 366) | |
| 5′-GAA AGC TGA GAT GGA GG-3′ | (SEQ. ID NO: 374) |
| (Fragment 367) | |
| 5′-GAA AGC TGA GAT GGA G-3′ | (SEQ. ID NO: 375) |
| (Fragment 368) | |
| 5′-GAA AGC TGA GAT GGA-3′ | (SEQ. ID NO: 376) |
| (Fragment 369) | |
| 5′-GAA AGC TGA GAT GG-3′ | (SEQ. ID NO: 377) |
| (Fragment 370) | |
| 5′-GAA AGC TGA GAT G-3′ | (SEQ. ID NO: 378) |
| (Fragment 371) | |
| 5′-GAA AGC TGA GAT-3′ | (SEQ. ID NO: 379) |
| (Fragment 372) | |
| 5′-GAA AGC TGA GA-3′ | (SEQ. ID NO: 380) |
| (Fragment 373) | |
| 5′-GAA AGC TGA G-3′ | (SEQ. ID NO: 381) |
| (Fragment 374) | |
| 5′-AA AGC TGA GAT GGA GGG CGG | (SEQ. ID NO: 382) |
| CAT GGC GGG CAC AGG CTG GGC-3′ | |
| (Fragment 375) | |
| 5′-AA AGC TGA GAT GGA GGG CGG | (SEQ. ID NO: 383) |
| CAT GGC GGG GAG AGG CTG GG-3′ | |
| (Fragment 376) | |
| 5′-AA AGC TGA GAT GGA GGG CGG | (SEQ. ID NO: 384) |
| CAT GGC GGG GAG AGG CTG G-3′ | |
| (Fragment 377) | |
| 5′-AA AGC TGA GAT GGA GGG CGG | (SEQ. ID NO: 385) |
| CAT GGC GGG GAG AGG GTG-3′ | |
| (Fragment 378) | |
| 5′-AA AGG TGA GAT GGA GGG CGG | (SEQ. ID NO: 386) |
| CAT GGC GGG GAG AGG CT-3′ | |
| (Fragment 379) | |
| 5′-AA AGC TGA GAT GGA GGG CGG | (SEQ. ID NO: 387) |
| CAT GGC GGG GAG AGG G-3′ | |
| (Fragment 380) | |
| 5′-AA AGC TGA GAT GGA GGG CGG | (SEQ. ID NO: 388) |
| CAT GGC GGG GAG AGG-3′ | |
| (Fragment 381) | |
| 5′-AA AGG TGA GAT GGA GGG CGG | (SEQ. ID NO: 389) |
| CAT GGG GGG GAG AG-3′ | |
| (Fragment 382) | |
| 5′-AA AGG TGA GAT GGA GGG GGG | (SEQ. ID NO: 390) |
| CAT GGC GGG GAG A-3′ | |
| (Fragment 383) | |
| 5′-AA AGC TGA GAT GGA GGG GGG | (SEQ. ID NO: 391) |
| CAT GGC GGG CAC-3′ | |
| (Fragment 384) | |
| 5′-AA AGC TGA GAT GGA GGG CGG | (SEQ. ID NO: 392) |
| CAT GGG GGG GA-3′ (Fragment 385) | |
| 5′-AA AGC TGA GAT GGA GGG CGG | (SEQ. ID NO: 393) |
| CAT GGC GGG C-3′ (Fragment 386) | |
| 5′-AA AGC TGA GAT GGA GGG CGG | (SEQ. ID NO: 394) |
| CAT GGC GGG-3′ (Fragment 387) | |
| 5′-AA AGC TGA GAT GGA GGG CGG | (SEQ. ID NO: 395) |
| CAT GGC GG-3′ (Fragment 388) | |
| 5′-AA AGC TGA GAT GGA GGG CGG | (SEQ. ID NO: 396) |
| CAT GGC G-3′ (Fragment 389) | |
| 5′-AA AGC TGA GAT GGA GGG CGG | (SEQ. ID NO: 397) |
| CAT GGC-3′ (Fragment 390) | |
| 5′-AA AGC TGA GAT GGA GGG CGG | (SEQ. ID NO: 398) |
| CAT GG-3′ (Fragment 391) | |
| 5′-AA AGC TGA GAT GGA GGG CGG | (SEQ. ID NO: 399) |
| CAT G-3′ (Fragment 392) | |
| 5′-AA AGC TGA GAT GGA GGG CGG | (SEQ. ID NO: 400) |
| CAT-3′ (Fragment 393) | |
| 5′-AA AGC TGA GAT GGA GGG CGG | (SEQ. ID NO: 401) |
| CA-3′ (Fragment 394) | |
| 5′-AA AGC TGA GAT GGA GGG CGG | (SEQ. ID NO: 402) |
| C-3′ (Fragment 395) | |
| 5′-AA AGC TGA GAT GGA GGG CGG-3′ | (SEQ. ID NO: 403) |
| (Fragment 396) | |
| 5′-AA AGC TGA GAT GGA GGG CG-3′ | (SEQ. ID NO: 404) |
| (Fragment 397) | |
| 5′-AA AGC TGA GAT GGA GGG C-3′ | (SEQ. ID NO: 405) |
| (Fragment 398) | |
| 5′-AA AGC TGA GAT GGA GGG-3′ | (SEQ. ID NO: 406) |
| (Fragment 399) | |
| 5′-AA AGC TGA GAT GGA GG-3′ | (SEQ. ID NO: 407) |
| (Fragment 400) | |
| 5′-AA AGC TGA GAT GGA G-3′ | (SEQ. ID NO: 408) |
| (Fragment 401) | |
| 5′-AA AGC TGA GAT GGA-3′ | (SEQ. ID NO: 409) |
| (Fragment 402) | |
| 5′-AA AGC TGA GAT GG-3′ | (SEQ. ID NO: 410) |
| (Fragment 403) | |
| 5′-AA AGC TGA GAT G-3′ | (SEQ. ID NO: 411) |
| (Fragment 404) | |
| 5′-AA AGC TGA GAT-3′ | (SEQ. ID NO: 412) |
| (Fragment 405) | |
| 5′-AA AGC TGA GA-3′ | (SEQ. ID NO: 413) |
| (Fragment 406) | |
| 5′-A AGC TGA GAT GGA GGG CGG CAT | (SEQ. ID NO: 414) |
| GGC GGG CAC AGG CTG GGC-3′ | |
| (Fragment 407) | |
| 5′-A AGC TGA GAT GGA GGG CGG CAT | (SEQ. ID NO: 415) |
| GGC GGG CAC AGG CTG GG-3′ | |
| (Fragment 408) | |
| 5′-A AGC TGA GAT GGA GGG CGG CAT | (SEQ. ID NO: 416) |
| GGC GGG CAC AGG CTG G-3′ | |
| (Fragment 409) | |
| 5′-A AGC TGA GAT GGA GGG CGG CAT | (SEQ. ID NO: 417) |
| GGC GGG CAC AGG CTG-3′ | |
| (Fragment 410) | |
| 5′-A AGC TGA GAT GGA GGG CGG CAT | (SEQ. ID NO: 418) |
| GGC GGG CAC AGG CT-3′ | |
| (Fragment 411) | |
| 5′-A AGC TGA GAT GGA GGG CGG CAT | (SEQ. ID NO: 419) |
| GGC GGG CAC AGG C-3′ | |
| (Fragment 412) | |
| 5′-A AGC TGA GAT GGA GGG CGG CAT | (SEQ. ID NO: 420) |
| GGC GGG CAC AGG-3′ | |
| (Fragment 413) | |
| 5′-A AGC TGA GAT GGA GGG CGG CAT | (SEQ. ID NO: 421) |
| GGC GGG CAC AG-3′ (Fragment 414) | |
| 5′-A AGC TGA GAT GGA GGG CGG CAT | (SEQ. ID NO: 422) |
| GGC GGG CAC A-3′ (Fragment 415) | |
| 5′-A AGC TGA GAT GGA GGG CGG CAT | (SEQ. ID NO: 423) |
| GGC GGG CAC-3′ (Fragment 416) | |
| 5′-A AGC TGA GAT GGA GGG CGG CAT | (SEQ. ID NO: 424) |
| GGC GGG CA-3′ (Fragment 417) | |
| 5′-A AGC TGA GAT GGA GGG CGG CAT | (SEQ. ID NO: 425) |
| GGC GGG C-3′ (Fragment 418) | |
| 5′-A AGC TGA GAT GGA GGG CGG CAT | (SEQ. ID NO: 426) |
| GGC GGG-3′ (Fragment 419) | |
| 5′-A AGC TGA GAT GGA GGG CGG CAT | (SEQ. ID NO: 427) |
| GGC GG-3′ (Fragment 420) | |
| 5′-A AGC TGA GAT GGA GGG CGG CAT | (SEQ. ID NO: 428) |
| GGC G-3′ (Fragment 421) | |
| 5′-A AGC TGA GAT GGA GGG CGG CAT | (SEQ. ID NO: 429) |
| GGC-3′ (Fragment 422) | |
| 5′-A AGC TGA GAT GGA GGG CGG CAT | (SEQ. ID NO: 430) |
| GG-3′ (Fragment 423) | |
| 5′-A AGC TGA GAT GGA GGG CGG CAT | (SEQ. ID NO: 431) |
| G-3′ (Fragment 424) | |
| 5′-A AGC TGA GAT GGA GGG CGG | (SEQ. ID NO: 432) |
| CAT-3′ (Fragment 425) | |
| 5′-A AGC TGA GAT GGA GGG CGG | (SEQ. ID NO: 433) |
| GA-3′ (Fragment 426) | |
| 5′-A AGG TGA GAT GGA GGG GGG | (SEQ. ID NO: 434) |
| C-3′ (Fragment 427) | |
| 5′-A AGC TGA GAT GGA GGG GGG-3′ | (SEQ. ID NO: 435) |
| (Fragment 428) | |
| 5′-A AGC TGA GAT GGA GGG GG-3′ | (SEQ. ID NO: 436) |
| (Fragment 429) | |
| 5′-A AGG TGA GAT GGA GGG C-3′ | (SEQ. ID NO: 437) |
| (Fragment 430) | |
| 5′-A AGC TGA GAT GGA GGG-3′ | (SEQ. ID NO: 438) |
| (Fragment 431) | |
| 5′-A AGC TGA GAT GGA GG-3′ | (SEQ. ID NO: 439) |
| (Fragment 432) | |
| 5′-A AGC TGA GAT GGA G-3′ | (SEQ. ID NO: 440) |
| (Fragment 433) | |
| 5′-A AGC TGA GAT GGA-3′ | (SEQ. ID NO: 441) |
| (Fragment 434) | |
| 5′-A AGC TGA GAT GG-3′ | (SEQ. ID NO: 442) |
| (Fragment 435) | |
| 5′-A AGC TGA GAT G-3′ | (SEQ. ID NO: 443) |
| (Fragment 436) | |
| 5′-A AGC TGA GAT-3′ | (SEQ. ID NO: 444) |
| (Fragment 437) | |
| 5′-AGC TGA GAT GGA GGG CGG CAT | (SEQ. ID NO: 445) |
| GGC GGG GAG AGG CTG GGC-3′ | |
| (Fragment 438) | |
| 5′-AGC TGA GAT GGA GGG CGG CAT | (SEQ. ID NO: 446) |
| GGC GGG GAG AGG CTG GG-3′ | |
| (Fragment 439) | |
| 5′-AGC TGA GAT GGA GGG CGG CAT | (SEQ. ID NO: 447) |
| GGC GGG GAG AGG CTG G-3′ | |
| (Fragment 440) | |
| 5′-AGC TGA GAT GGA GGG GGG CAT | (SEQ. ID NO: 448) |
| GGC GGG GAG AGG GTG-3′ | |
| (Fragment 441) | |
| 5′-AGG TGA GAT GGA GGG GGG CAT | (SEQ. ID NO: 449) |
| GGC GGG GAG AGG GT-3′ | |
| (Fragment 442) | |
| 5′-AGG TGA GAT GGA GGG CGG CAT | (SEQ. ID NO: 450) |
| GGC GGG GAG AGG C-3′ | |
| (Fragment 443) | |
| 5′-AGC TGA GAT GGA GGG CGG CAT | (SEQ. ID NO: 451) |
| GGC GGG GAG AGG-3′ | |
| (Fragment 444) | |
| 5′-AGC TGA GAT GGA GGG CGG CAT | (SEQ. ID NO: 452) |
| GGC GGG GAG AG-3′ (Fragment 445) | |
| 5′-AGC TGA GAT GGA GGG CGG CAT | (SEQ. ID NO: 453) |
| GGC GGG GAG A-3′ (Fragment 446) | |
| 5′-AGC TGA GAT GGA GGG CGG CAT | (SEQ. ID NO: 454) |
| GGC GGG CAC-3′ (Fragment 447) | |
| 5′-AGG TGA GAT GGA GGG CGG CAT | (SEQ. ID NO: 455) |
| GGC GGG CA-3′ (Fragment 448) | |
| 5′-AGC TGA GAT GGA GGG CGG CAT | (SEQ. ID NO: 456) |
| GGC GGG C-3′ (Fragment 449) | |
| 5′-AGC TGA GAT GGA GGG CGG CAT | (SEQ. ID NO: 457) |
| GGC GGG-3′ (Fragment 450) | |
| 5′-AGC TGA GAT GGA GGG CGG CAT | (SEQ. ID NO: 458) |
| GGC GG-3′ (Fragment 451) | |
| 5′-AGC TGA GAT GGA GGG CGG CAT | (SEQ. ID NO: 459) |
| GGC G-3′ (Fragment 452) | |
| 5′-AGC TGA GAT GGA GGG CGG CAT | (SEQ. ID NO: 460) |
| GGC-3′ (Fragment 453) | |
| 5′-AGG TGA GAT GGA GGG CGG CAT | (SEQ. ID NO: 461) |
| GG-3′ (Fragment 454) | |
| 5′-AGC TGA GAT GGA GGG CGG CAT | (SEQ. ID NO: 462) |
| G-3′ (Fragment 455) | |
| 5′-AGG TGA GAT GGA GGG CGG | (SEQ. ID NO: 463) |
| CAT-3′ (Fragment 456) | |
| 5′-AGG TGA GAT GGA GGG CGG CA-3′ | (SEQ. ID NO: 464) |
| (Fragment 457) | |
| 5′-AGG TGA GAT GGA GGG CGG C-3′ | (SEQ. ID NO: 465) |
| (Fragment 458) | |
| 5′-AGG TGA GAT GGA GGG GGG-3′ | (SEQ. ID NO: 466) |
| (Fragment 459) | |
| 5′-AGC TGA GAT GGA GGG GG-3′ | (SEQ. ID NO: 467) |
| (Fragment 460) | |
| 5′-AGC TGA GAT GGA GGG C-3′ | (SEQ. ID NO: 468) |
| (Fragment 461) | |
| 5′-AGC TGA GAT GGA GGG-3′ | (SEQ. ID NO: 469) |
| (Fragment 462) | |
| 5′-AGC TGA GAT GGA GG-3′ | (SEQ. ID NO: 470) |
| (Fragment 463) | |
| 5′-AGG TGA GAT GGA G-3′ | (SEQ. ID NO: 471) |
| (Fragment 464) | |
| 5′-AGC TGA GAT GGA-3′ | (SEQ. ID NO: 472) |
| (Fragment 465) | |
| 5′-AGC TGA GAT GG-3′ | (SEQ. ID NO: 473) |
| (Fragment 466) | |
| 5′-AGC TGA GAT G-3′ | (SEQ. ID NO: 474) |
| (Fragment 467) | |
| 5′-GC TGA GAT GGA GGG CGG CAT | (SEQ. ID NO: 475) |
| GGC GGG CAC AGG CTG GGC-3′ | |
| (Fragment 468) | |
| 5′-GC TGA GAT GGA GGG CGG CAT | (SEQ. ID NO: 476) |
| GGC GGG CAC AGG CTG GG-3′ | |
| (Fragment 469) | |
| 5′-GC TGA GAT GGA GGG CGG CAT | (SEQ. ID NO: 477) |
| GGC GGG CAC AGG CTG G-3′ | |
| (Fragment 470) | |
| 5′-GC TGA GAT GGA GGG CGG CAT | (SEQ. ID NO: 478) |
| GGC GGG CAC AGG CTG-3′ | |
| (Fragment 471) | |
| 5′-GC TGA GAT GGA GGG CGG CAT | (SEQ. ID NO: 479) |
| GGC GGG CAC AGG CT-3′ | |
| (Fragment 472) | |
| 5′-GC TGA GAT GGA GGG CGG CAT | (SEQ. ID NO: 480) |
| GGC GGG CAC AGG C-3′ | |
| (Fragment 473) | |
| 5′-GC TGA GAT GGA GGG CGG CAT | (SEQ. ID NO: 481) |
| GGC GGG CAC AGG-3′ | |
| (Fragment 474) | |
| 5′-GC TGA GAT GGA GGG CGG CAT | (SEQ. ID NO: 482) |
| GGC GGG CAC AG-3′ (Fragment 475) | |
| 5′-GC TGA GAT GGA GGG CGG CAT | (SEQ. ID NO: 483) |
| GGC GGG CAC A-3′ (Fragment 476) | |
| 5′-GC TGA GAT GGA GGG CGG CAT | (SEQ. ID NO: 484) |
| GGC GGG CAC-3′ (Fragment 477) | |
| 5′-GC TGA GAT GGA GGG CGG CAT | (SEQ. ID NO: 485) |
| GGC GGG CA-3′ (Fragment 478) | |
| 5′-GC TGA GAT GGA GGG CGG CAT | (SEQ. ID NO: 486) |
| GGC GGG C-3′ (Fragment 479) | |
| 5′-GC TGA GAT GGA GGG CGG CAT | (SEQ. ID NO: 487) |
| GGC GGG-3′ (Fragment 480) | |
| 5′-GC TGA GAT GGA GGG CGG CAT | (SEQ. ID NO: 488) |
| GGC GG-3′ (Fragment 481) | |
| 5′-GC TGA GAT GGA GGG CGG CAT | (SEQ. ID NO: 489) |
| GGC G-3′ (Fragment 482) | |
| 5′-GC TGA GAT GGA GGG CGG CAT | (SEQ. ID NO: 490) |
| GGC-3′ (Fragment 483) | |
| 5′-GC TGA GAT GGA GGG CGG CAT | (SEQ. ID NO: 491) |
| GG-3′ (Fragment 484) | |
| 5′-GC TGA GAT GGA GGG CGG CAT | (SEQ. ID NO: 492) |
| G-3′ (Fragment 485) | |
| 5′-GC TGA GAT GGA GGG CGG CAT-3′ | (SEQ. ID NO: 493) |
| (Fragment 486) | |
| 5′-GC TGA GAT GGA GGG CGG CA-3′ | (SEQ. ID NO: 494) |
| (Fragment 487) | |
| 5′-GC TGA GAT GGA GGG CGG C-3′ | (SEQ. ID NO: 495) |
| (Fragment 488) | |
| 5′-GC TGA GAT GGA GGG CGG-3′ | (SEQ. ID NO: 496) |
| (Fragment 489) | |
| 5′-GC TGA GAT GGA GGG CG-3′ | (SEQ. ID NO: 497) |
| (Fragment 490) | |
| 5′-GC TGA GAT GGA GGG C-3′ | (SEQ. ID NO: 498) |
| (Fragment 491) | |
| 5′-GC TGA GAT GGA GGG-3′ | (SEQ. ID NO: 499) |
| (Fragment 492) | |
| 5′-GC TGA GAT GGA GG-3′ | (SEQ. ID NO: 500) |
| (Fragment 493) | |
| 5′-GC TGA GAT GGA G-3′ | (SEQ. ID NO: 501) |
| (Fragment 494) | |
| 5′-GC TGA GAT GGA-3′ | (SEQ. ID NO: 502) |
| (Fragment 495) | |
| 5′-GC TGA GAT GG-3′ | (SEQ. ID NO: 503) |
| (Fragment 496) | |
| 5′-C TGA GAT GGA GGG CGG CAT GGC | (SEQ. ID NO: 504) |
| GGG CAC AGG CTG GGC-3′ | |
| (Fragment 497) | |
| 5′-C TGA GAT GGA GGG CGG CAT GGC | (SEQ. ID NO: 505) |
| GGG CAC AGG CTG GG-3′ | |
| (Fragment 498) | |
| 5′-C TGA GAT GGA GGG CGG CAT GGC | (SEQ. ID NO: 506) |
| GGG CAC AGG CTG G-3′ | |
| (Fragment 499) | |
| 5′-C TGA GAT GGA GGG CGG CAT GGC | (SEQ. ID NO: 507) |
| GGG CAC AGG CTG-3′ | |
| (Fragment 500) | |
| 5′-C TGA GAT GGA GGG CGG CAT GGC | (SEQ. ID NO: 508) |
| GGG CAC AGG CT-3′ (Fragment 501) | |
| 5′-C TGA GAT GGA GGG CGG CAT GGC | (SEQ. ID NO: 509) |
| GGG CAC AGG C-3′ (Fragment 502) | |
| 5′-C TGA GAT GGA GGG CGG CAT GGC | (SEQ. ID NO: 5 10) |
| GGG CAC AGG-3′ (Fragment 503) | |
| 5′-C TGA GAT GGA GGG CGG CAT GGC | (SEQ. ID NO: 511) |
| GGG CAC AG-3′ (Fragment 504) | |
| 5′-C TGA GAT GGA GGG CGG CAT GGC | (SEQ. ID NO: 512) |
| GGG CAC A-3′ (Fragment 505) | |
| 5′-C TGA GAT GGA GGG CGG CAT GGC | (SEQ. ID NO: 513) |
| GGG CAC-3′ (Fragment 506) | |
| 5′-C TGA GAT GGA GGG CGG CAT GGC | (SEQ. ID NO: 514) |
| GGG CA-3′ (Fragment 507) | |
| 5′-C TGA GAT GGA GGG CGG CAT GGC | (SEQ. ID NO: 515) |
| GGG C-3′ (Fragment 508) | |
| 5′-C TGA GAT GGA GGG CGG CAT GGC | (SEQ. ID NO: 516) |
| GGG-3′ (Fragment 509) | |
| 5′-C TGA GAT GGA GGG CGG CAT GGC | (SEQ. ID NO: 517) |
| GG-3′ (Fragment 510) | |
| 5′-C TGA GAT GGA GGG CGG CAT GGC | (SEQ. ID NO: 518) |
| G-3′ (Fragment 511) | |
| 5′-C TGA GAT GGA GGG CGG CAT | (SEQ. ID NO: 519) |
| GGC-3′ (Fragment 512) | |
| 5′-C TGA GAT GGA GGG CGG CAT | (SEQ. ID NO: 520) |
| GG-3′ (Fragment 513) | |
| 5′-C TGA GAT GGA GGG CGG CAT | (SEQ. ID NO: 521) |
| G-3′ (Fragment 514) | |
| 5′-C TGA GAT GGA GGG CGG CAT-3′ | (SEQ. ID NO: 522) |
| (Fragment 515) | |
| 5′-C TGA GAT GGA GGG CGG CA-3′ | (SEQ. ID NO: 523) |
| (Fragment 516) | |
| 5′-C TGA GAT GGA GGG CGG C-3′ | (SEQ. ID NO: 524) |
| (Fragment 517) | |
| 5′-C TGA GAT GGA GGG CGG-3′ | (SEQ. ID NO: 525) |
| (Fragment 518) | |
| 5′-C TGA GAT GGA GGG CG-3′ | (SEQ. ID NO: 526) |
| (Fragment 519) | |
| 5′-C TGA GAT GGA GGG C-3′ | (SEQ. ID NO: 527) |
| (Fragment 520) | |
| 5′-C TGA GAT GGA GGG-3′ | (SEQ. ID NO: 528) |
| (Fragment 521) | |
| 5′-C TGA GAT GGA GG-3′ | (SEQ. ID NO: 529) |
| (Fragment 522) | |
| 5′-C TGA GAT GGA G-3′ | (SEQ. ID NO: 530) |
| (Fragment 523) | |
| 5′-C TGA GAT GGA-3′ | (SEQ. ID NO: 531) |
| (Fragment 524) | |
| 5′-TGA GAT GGA GGG CGG CAT GGC | (SEQ. ID NO: 532) |
| GGG CAC AGG CTG GGC-3′ | |
| (Fragment 525) | |
| 5′-TGA GAT GGA GGG CGG CAT GGC | (SEQ. ID NO: 533) |
| GGG CAC AGG CTG GG-3′ | |
| (Fragment 526) | |
| 5′-TGA GAT GGA GGG CGG CAT GGC | (SEQ. ID NO: 534) |
| GGG CAC AGG CTG G-3′ | |
| (Fragment 527) | |
| 5′-TGA GAT GGA GGG CGG CAT GGC | (SEQ. ID NO: 535) |
| GGG CAC AGG CTG-3′ | |
| (Fragment 528) | |
| 5′-TGA GAT GGA GGG CGG CAT GGC | (SEQ. ID NO: 536) |
| GGG CAC AGG CT-3′ (Fragment 529) | |
| 5′-TGA GAT GGA GGG CGG CAT GGC | (SEQ. ID NO: 537) |
| GGG CAC AGG C-3′ (Fragment 530) | |
| 5′-TGA GAT GGA GGG CGG CAT GGC | (SEQ. ID NO: 538) |
| GGG CAC AGG-3′ (Fragment 531) | |
| 5′-TGA GAT GGA GGG CGG CAT GGC | (SEQ. ID NO: 539) |
| GGG CAC AG-3′ (Fragment 532) | |
| 5′-TGA GAT GGA GGG CGG CAT GGC | (SEQ. ID NO: 540) |
| GGG CAC A-3′ (Fragment 533) | |
| 5′-TGA GAT GGA GGG CGG CAT GGC | (SEQ. ID NO: 541) |
| GGG CAC-3′ (Fragment 534) | |
| 5′-TGA GAT GGA GGG CGG CAT GGC | (SEQ. ID NO: 542) |
| GGG CA-3′ (Fragment 535) | |
| 5′-TGA GAT GGA GGG CGG CAT GGC | (SEQ. ID NO: 543) |
| GGG C-3′ (Fragment 536) | |
| 5′-TGA GAT GGA GGG CGG CAT GGC | (SEQ. ID NO: 544) |
| GGG-3′ (Fragment 537) | |
| 5′-TGA GAT GGA GGG CGG CAT GGC | (SEQ. ID NO: 545) |
| GG-3′ (Fragment 538) | |
| 5′-TGA GAT GGA GGG CGG CAT GGC | (SEQ. ID NO: 546) |
| G-3′ (Fragment 539) | |
| 5′-TGA GAT GGA GGG CGG CAT | (SEQ. ID NO: 547) |
| GGC-3′ (Fragment 540) | |
| 5′-TGA GAT GGA GGG CGG CAT GG-3′ | (SEQ. ID NO: 548) |
| (Fragment 541) | |
| 5′-TGA GAT GGA GGG CGG CAT G-3′ | (SEQ. ID NO: 549) |
| (Fragment 542) | |
| 5′-TGA GAT GGA GGG CGG CAT-3′ | (SEQ. ID NO: 550) |
| (Fragment 543) | |
| 5′-TGA GAT GGA GGG CGG CA-3′ | (SEQ. ID NO: 551) |
| (Fragment 544) | |
| 5′-TGA GAT GGA GGG CGG C-3′ | (SEQ. ID NO: 552) |
| (Fragment 545) | |
| 5′-TGA GAT GGA GGG CGG-3′ | (SEQ. ID NO: 553) |
| (Fragment 546) | |
| 5′-TGA GAT GGA GGG CG-3′ | (SEQ. ID NO: 554) |
| (Fragment 547) | |
| 5′-TGA GAT GGA GGG C-3′ | (SEQ. ID NO: 555) |
| (Fragment 548) | |
| 5′-TGA GAT GGA GGG-3′ | (SEQ. ID NO: 556) |
| (Fragment 549) | |
| 5′-TGA GAT GGA GG-3′ | (SEQ. ID NO: 557) |
| (Fragment 550) | |
| 5′-TGA GAT GGA G-3′ | (SEQ. ID NO: 558) |
| (Fragment 551) | |
| 5′-GA GAT GGA GGG CGG CAT GGC | (SEQ. ID NO: 559) |
| GGG GAG AGG CTG GGC-3′ | |
| (Fragment 552) | |
| 5′-GA GAT GGA GGG CGG CAT GGC | (SEQ. ID NO: 560) |
| GGG CAC AGG CTG GG-3′ | |
| (Fragment 553) | |
| 5′-GA GAT GGA GGG CGG CAT GGC | (SEQ. ID NO: 561) |
| GGG CAC AGG CTG G-3′ | |
| (Fragment 554) | |
| 5′-GA GAT GGA GGG CGG CAT GGC | (SEQ. ID NO: 562) |
| GGG CAC AGG CTG-3′ | |
| (Fragment 555) | |
| 5′-GA GAT GGA GGG CGG CAT GGC | (SEQ. ID NO: 563) |
| GGG CAC AGG CT-3′ (Fragment 556) | |
| 5′-GA GAT GGA GGG CGG CAT GGC | (SEQ. ID NO: 564) |
| GGG CAC AGG C-3′ (Fragment 557) | |
| 5′-GA GAT GGA GGG CGG CAT GGC | (SEQ. ID NO: 565) |
| GGG CAC AGG-3′ (Fragment 558) | |
| 5′-GA GAT GGA GGG CGG CAT GGC | (SEQ. ID NO: 566) |
| GGG CAC AG-3′ (Fragment 559) | |
| 5′-GA GAT GGA GGG CGG CAT GGC | (SEQ. ID NO: 567) |
| GGG CAC A-3′ (Fragment 560) | |
| 5′-GA GAT GGA GGG CGG CAT GGC | (SEQ. ID NO: 568) |
| GGG CAC-3′ (Fragment 561) | |
| 5′-GA GAT GGA GGG CGG CAT GGC | (SEQ. ID NO: 569) |
| GGG CA-3′ (Fragment 562) | |
| 5′-GA GAT GGA GGG CGG CAT GGC | (SEQ. ID NO: 570) |
| GGG C-3′ (Fragment 563) | |
| 5′-GA GAT GGA GGG CGG CAT GGC | (SEQ. ID NO: 571) |
| GGG-3′ (Fragment 564) | |
| 5′-GA GAT GGA GGG CGG CAT GGC | (SEQ. ID NO: 572) |
| GG-3′ (Fragment 565) | |
| 5′-GA GAT GGA GGG CGG CAT GGC | (SEQ. ID NO: 573) |
| G-3′ (Fragment 566) | |
| 5′-GA GAT GGA GGG CGG CAT GGC-3′ | (SEQ. ID NO: 574) |
| (Fragment 567) | |
| 5′-GA GAT GGA GGG CGG CAT GG-3′ | (SEQ. ID NO: 575) |
| (Fragment 568) | |
| 5′-GA GAT GGA GGG CGG CAT G-3′ | (SEQ. ID NO: 576) |
| (Fragment 569) | |
| 5′-GA GAT GGA GGG CGG CAT-3′ | (SEQ. ID NO: 577) |
| (Fragment 570) | |
| 5′-GA GAT GGA GGG CGG CA-3′ | (SEQ. ID NO: 578) |
| (Fragment 571) | |
| 5′-GA GAT GGA GGG CGG C-3′ | (SEQ. ID NO: 579) |
| (Fragment 572) | |
| 5′-GA GAT GGA GGG CGG-3′ | (SEQ. ID NO: 580) |
| (Fragment 573) | |
| 5′-GA GAT GGA GGG CG-3′ | (SEQ. ID NO: 581) |
| (Fragment 574) | |
| 5′-GA GAT GGA GGG C-3′ | (SEQ. ID NO: 582) |
| (Fragment 575) | |
| 5′-GA GAT GGA GGG-3′ | (SEQ. ID NO: 583) |
| (Fragment 576) | |
| 5′-GA GAT GGA GG-3′ | (SEQ. ID NO: 584) |
| (Fragment 577) | |
| 5′-A GAT GGA GGG CGG CAT GGC GGG | (SEQ. ID NO: 585) |
| CAC AGG CTG GGC-3′ | |
| (Fragment 578) | |
| 5′-A GAT GGA GGG CGG CAT GGC GGG | (SEQ. ID NO: 586) |
| CAC AGG CTG GG-3′ (Fragment 579) | |
| 5′-A GAT GGA GGG CGG CAT GGC GGG | (SEQ. ID NO: 587) |
| CAC AGG CTG G-3′ (Fragment 580) | |
| 5′-A GAT GGA GGG CGG CAT GGC GGG | (SEQ. ID NO: 588) |
| CAC AGG CTG-3′ (Fragment 581) | |
| 5′-A GAT GGA GGG CGG CAT GGC GGG | (SEQ. ID NO: 589) |
| CAC AGG CT-3′ (Fragment 582) | |
| 5′-A GAT GGA GGG CGG CAT GGC GGG | (SEQ. ID NO: 590) |
| CAC AGG C-3′ (Fragment 583) | |
| 5′-A GAT GGA GGG CGG CAT GGC GGG | (SEQ. ID NO: 591) |
| CAC AGG-3′ (Fragment 584) | |
| 5′-A GAT GGA GGG CGG CAT GGC GGG | (SEQ. ID NO: 592) |
| CAC AG-3′ (Fragment 585) | |
| 5′-A GAT GGA GGG CGG CAT GGC GGG | (SEQ. ID NO: 593) |
| CAC A-3′ (Fragment 586) | |
| 5′-A GAT GGA GGG CGG CAT GGC GGG | (SEQ. ID NO: 594) |
| CAC-3′ (Fragment 587) | |
| 5′-A GAT GGA GGG CGG CAT GGC GGG | (SEQ. ID NO: 595) |
| CA-3′ (Fragment 588) | |
| 5′-A GAT GGA GGG CGG CAT GGC GGG | (SEQ. ID NO: 596) |
| C-3′ (Fragment 589) | |
| 5′-A GAT GGA GGG CGG CAT GGC | (SEQ. ID NO: 597) |
| GGG-3′ (Fragment 590) | |
| 5′-A GAT GGA GGG CGG CAT GGC | (SEQ. ID NO: 598) |
| GG-3′ (Fragment 591) | |
| 5′-A GAT GGA GGG CGG CAT GGC | (SEQ. ID NO: 599) |
| G-3′ (Fragment 592) | |
| 5′-A GAT GGA GGG CGG CAT GGC-3′ | (SEQ. ID NO: 600) |
| (Fragment 593) | |
| 5′-A GAT GGA GGG CGG CAT GG-3′ | (SEQ. ID NO: 601) |
| (Fragment 594) | |
| 5′-A GAT GGA GGG CGG CAT G-3′ | (SEQ. ID NO: 602) |
| (Fragment 595) | |
| 5′-A GAT GGA GGG CGG CAT-3′ | (SEQ. ID NO: 603) |
| (Fragment 596) | |
| 5′-A GAT GGA GGG CGG CA-3′ | (SEQ. ID NO: 604) |
| (Fragment 597) | |
| 5′-A GAT GGA GGG CGG C-3′ | (SEQ. ID NO: 605) |
| (Fragment 598) | |
| 5′-A GAT GGA GGG CGG-3′ | (SEQ. ID NO: 606) |
| (Fragment 599) | |
| 5′-A GAT GGA GGG CG-3′ | (SEQ. ID NO: 607) |
| (Fragment 600) | |
| 5′-A GAT GGA GGG C-3′ | (SEQ. ID NO: 608) |
| (Fragment 601) | |
| 5′-A GAT GGA GGG-3′ | (SEQ. ID NO: 609) |
| (Fragment 602) | |
| 5′-GAT GGA GGG CGG CAT GGC GGG | (SEQ. ID NO: 610) |
| CAC AGG CTG GGC-3′ | |
| (Fragment 603) | |
| 5′-GAT GGA GGG CGG CAT GGC GGG | (SEQ. ID NO: 611) |
| CAC AGG CTG GG-3′ (Fragment 604) | |
| 5′-GAT GGA GGG CGG CAT GGC GGG | (SEQ. ID NO: 612) |
| CAC AGG CTG G-3′ (Fragment 605) | |
| 5′-GAT GGA GGG CGG CAT GGC GGG | (SEQ. ID NO: 613) |
| CAC AGG CTG-3′ (Fragment 606) | |
| 5′-GAT GGA GGG CGG CAT GGC GGG | (SEQ. ID NO: 614) |
| CAC AGG CT-3′ (Fragment 607) | |
| 5′-GAT GGA GGG CGG CAT GGC GGG | (SEQ. ID NO: 615) |
| CAC AGG C-3′ (Fragment 608) | |
| 5′-GAT GGA GGG CGG CAT GGC GGG | (SEQ. ID NO: 616) |
| CAC AGG-3′ (Fragment 609) | |
| 5′-GAT GGA GGG CGG CAT GGC GGG | (SEQ. ID NO: 617) |
| CAC AG-3′ (Fragment 610) | |
| 5′-GAT GGA GGG CGG CAT GGC GGG | (SEQ. ID NO: 618) |
| CAC A-3′ (Fragment 611) | |
| 5′-GAT GGA GGG CGG CAT GGC GGG | (SEQ. ID NO: 619) |
| CAC-3′ (Fragment 612) | |
| 5′-GAT GGA GGG CGG CAT GGC GGG | (SEQ. ID NO: 620) |
| CA-3′ (Fragment 613) | |
| 5′-GAT GGA GGG CGG CAT GGC GGG | (SEQ. ID NO: 621) |
| C-3′ (Fragment 614) | |
| 5′-GAT GGA GGG CGG CAT GGC | (SEQ. ID NO: 622) |
| GGG-3′ (Fragment 615) | |
| 5′-GAT GGA GGG CGG CAT GGC GG-3′ | (SEQ. ID NO: 623) |
| (Fragment 616) | |
| 5′-GAT GGA GGG CGG CAT GGC G-3′ | (SEQ. ID NO: 624) |
| (Fragment 617) | |
| 5′-GAT GGA GGG CGG CAT GGC-3′ | (SEQ. ID NO: 625) |
| (Fragment 618) | |
| 5′-GAT GGA GGG CGG CAT GG-3′ | (SEQ. ID NO: 626) |
| (Fragment 619) | |
| 5′-GAT GGA GGG CGG CAT G-3′ | (SEQ. ID NO: 627) |
| (Fragment 620) | |
| 5′-GAT GGA GGG CGG CAT-3′ | (SEQ. ID NO: 628) |
| (Fragment 621) | |
| 5′-GAT GGA GGG CGG CA-3′ | (SEQ. ID NO: 629) |
| (Fragment 622) | |
| 5′-GAT GGA GGG CGG C-3′ | (SEQ. ID NO: 630) |
| (Fragment 623) | |
| 5′-GAT GGA GGG CGG-3′ | (SEQ. ID NO: 631) |
| (Fragment 624) | |
| 5′-GAT GGA GGG CG-3′ | (SEQ. ID NO: 632) |
| (Fragment 625) | |
| 5′-GAT GGA GGG C-3′ | (SEQ. ID NO: 633) |
| (Fragment 626) | |
| 5′-AT GGA GGG CGG CAT GGC GGG | (SEQ. ID NO: 634) |
| CAC AGG CTG GGC-3′ | |
| (Fragment 627) | |
| 5′-AT GGA GGG CGG CAT GGC GGG | (SEQ. ID NO: 635) |
| CAC AGG CTG GG-3′ (Fragment 628) | |
| 5′-AT GGA GGG CGG CAT GGC GGG | (SEQ. ID NO: 636) |
| CAC AGG CTG G-3′ (Fragment 629) | |
| 5′-AT GGA GGG CGG CAT GGC GGG | (SEQ. ID NO: 637) |
| CAC AGG CTG-3′ (Fragment 630) | |
| 5′-AT GGA GGG CGG CAT GGC GGG | (SEQ. ID NO: 638) |
| CAC AGG CT-3′ (Fragment 631) | |
| 5′-AT GGA GGG CGG CAT GGC GGG | (SEQ. ID NO: 639) |
| CAC AGG C-3′ (Fragment 632) | |
| 5′-AT GGA GGG CGG CAT GGC GGG | (SEQ. ID NO: 640) |
| CAC AGG-3′ (Fragment 633) | |
| 5′-AT GGA GGG CGG CAT GGC GGG | (SEQ. ID NO: 641) |
| CAC AG-3′ (Fragment 634) | |
| 5′-AT GGA GGG CGG CAT GGC GGG | (SEQ. ID NO: 642) |
| GAG A-3′ (Fragment 635) | |
| 5′-AT GGA GGG CGG CAT GGC GGG | (SEQ. ID NO: 643) |
| CAC-3′ (Fragment 636) | |
| 5′-AT GGA GGG CGG CAT GGC GGG | (SEQ. ID NO: 644) |
| GA-3′ (Fragment 637) | |
| 5′-AT GGA GGG GGG CAT GGC GGG | (SEQ. ID NO: 645) |
| C-3′ (Fragment 638) | |
| 5′-AT GGA GGG CGG CAT GGC GGG-3′ | (SEQ. ID NO: 646) |
| (Fragment 639) | |
| 5′-AT GGA GGG GGG CAT GGC GG-3′ | (SEQ. ID NO: 647) |
| (Fragment 640) | |
| 5′-AT GGA GGG CGG CAT GGC G-3′ | (SEQ. ID NO: 648) |
| (Fragment 641) | |
| 5′-AT GGA GGG CGG CAT GGG-3′ | (SEQ. ID NO: 649) |
| (Fragment 642) | |
| 5′-AT GGA GGG CGG CAT GG-3′ | (SEQ. ID NO: 650) |
| (Fragment 643) | |
| 5′-AT GGA GGG CGG CAT G-3′ | (SEQ. ID NO: 651) |
| (Fragment 644) | |
| 5′-AT GGA GGG CGG CAT-3′ | (SEQ. ID NO: 652) |
| (Fragment 645) | |
| 5′-AT GGA GGG CGG CA-3′ | (SEQ. ID NO: 653) |
| (Fragment 646) | |
| 5′-AT GGA GGG CGG C-3′ | (SEQ. ID NO: 654) |
| (Fragment 647) | |
| 5′-AT GGA GGG GGG-3′ | (SEQ. ID NO: 655) |
| (Fragment 648) | |
| 5′-AT GGA GGG CG-3′ | (SEQ. ID NO: 656) |
| (Fragment 649) | |
| 5′-T GGA GGG GGG CAT GGC GGG GAG | (SEQ. ID NO: 657) |
| AGG CTG GGC-3′ (Fragment 650) | |
| 5′-T GGA GGG CGG CAT GGG GGG GAG | (SEQ. ID NO: 658) |
| AGG CTG GG-3′ (Fragment 651) | |
| 5′-T GGA GGG GGG CAT GGC GGG GAG | (SEQ. ID NO: 659) |
| AGG GTG G-3′ (Fragment 652) | |
| 5′-T GGA GGG CGG CAT GGC GGG GAG | (SEQ. ID NO: 660) |
| AGG GTG-3′ (Fragment 653) | |
| 5′-T GGA GGG CGG CAT GGC GGG GAG | (SEQ. ID NO: 661) |
| AGG CT-3′ (Fragment 654) | |
| 5′-T GGA GGG CGG CAT GGC GGG GAG | (SEQ. ID NO: 662) |
| AGG C-3′ (Fragment 655) | |
| 5′-T GGA GGG CGG CAT GGG GGG GAG | (SEQ. ID NO: 663) |
| AGG-3′ (Fragment 656) | |
| 5′-T GGA GGG CGG CAT GGG GGG GAG | (SEQ. ID NO: 664) |
| AG-3′ (Fragment 657) | |
| 5′-T GGA GGG GGG CAT GGC GGG GAG | (SEQ. ID NO: 665) |
| A-3′ (Fragment 658) | |
| 5′-T GGA GGG CGG CAT GGC GGG | (SEQ. ID NO: 666) |
| GAC-3′ (Fragment 659) | |
| 5′-T GGA GGG CGG CAT GGC GGG | (SEQ. ID NO: 667) |
| CA-3′ (Fragment 660) | |
| 5′-T GGA GGG GGG CAT GGC GGG | (SEQ. ID NO: 668) |
| C-3′ (Fragment 661) | |
| 5′-T GGA GGG CGG CAT GGC GGG-3′ | (SEQ. ID NO: 669) |
| (Fragment 662) | |
| 5′-T GGA GGG CGG CAT GGC GG-3′ | (SEQ. ID NO: 670) |
| (Fragment 663) | |
| 5′-T GGA GGG GGG CAT GGC G-3′ | (SEQ. ID NO: 671) |
| (Fragment 664) | |
| 5′-T GGA GGG CGG CAT GGC-3′ | (SEQ. ID NO: 672) |
| (Fragment 665) | |
| 5′-T GGA GGG CGG CAT GG-3′ | (SEQ. ID NO: 673) |
| (Fragment 666) | |
| 5′-T GGA GGG CGG CAT G-3′ | (SEQ. ID NO: 674) |
| (Fragment 667) | |
| 5′-T GGA GGG CGG CAT-3′ | (SEQ. ID NO: 675) |
| (Fragment 668) | |
| 5′-T GGA GGG CGG CA-3′ | (SEQ. ID NO: 676) |
| (Fragment 669) | |
| 5′-T GGA GGG CGG C-3′ | (SEQ. ID NO: 677) |
| (Fragment 670) | |
| 5′-T GGA GGG CGG-3′ | (SEQ. ID NO: 678) |
| (Fragment 671) | |
| 5′-GGA GGG CGG CAT GGC GGG GAG | (SEQ. ID NO: 679) |
| AGG CTG GGC-3′ (Fragment 672) | |
| 5′-GGA GGG CGG CAT GGC GGG GAG | (SEQ. ID NO: 680) |
| AGG CTG GG-3′ (Fragment 673) | |
| 5′-GGA GGG CGG CAT GGC GGG GAG | (SEQ. ID NO: 681) |
| AGG CTG G-3′ (Fragment 674) | |
| 5′-GGA GGG CGG CAT GGC GGG GAG | (SEQ. ID NO: 682) |
| AGG GTG-3′ (Fragment 675) | |
| 5′-GGA GGG CGG CAT GGC GGG GAG | (SEQ. ID NO: 683) |
| AGG CT-3′ (Fragment 676) | |
| 5′-GGA GGG CGG CAT GGC GGG GAG | (SEQ. ID NO: 684) |
| AGG C-3′ (Fragment 677) | |
| 5′-GGA GGG CGG CAT GGG GGG GAG | (SEQ. ID NO: 685) |
| AGG-3′ (Fragment 678) | |
| 5′-GGA GGG CGG CAT GGC GGG GAG | (SEQ. ID NO: 686) |
| AG-3′ (Fragment 679) | |
| 5′-GGA GGG CGG CAT GGG GGG GAG | (SEQ. ID NO: 687) |
| A-3′ (Fragment 680) | |
| 5′-GGA GGG CGG CAT GGG GGG | (SEQ. ID NO: 688) |
| GAC-3′ (Fragment 681) | |
| 5′-GGA GGG CGG CAT GGC GGG CA-3′ | (SEQ. ID NO: 689) |
| (Fragment 682) | |
| 5′-GGA GGG CGG CAT GGC GGG C-3′ | (SEQ. ID NO: 690) |
| (Fragment 683) | |
| 5′-GGA GGG CGG CAT GGC GGG-3′ | (SEQ. ID NO: 691) |
| (Fragment 684) | |
| 5′-GGA GGG CGG CAT GGC GG-3′ | (SEQ. ID NO: 692) |
| (Fragment 685) | |
| 5′-GGA GGG CGG CAT GGC G-3′ | (SEQ. ID NO: 693) |
| (Fragment 686) | |
| 5′-GGA GGG CGG CAT GGC-3′ | (SEQ. ID NO: 694) |
| (Fragment 687) | |
| 5′-GGA GGG CGG CAT GG-3′ | (SEQ. ID NO: 695) |
| (Fragment 688) | |
| 5′-GGA GGG CGG CAT G-3′ | (SEQ. ID NO: 696) |
| (Fragment 689) | |
| 5′-GGA GGG CGG CAT-3′ | (SEQ. ID NO: 697) |
| (Fragment 690) | |
| 5′-GGA GGG CGG CA-3′ | (SEQ. ID NO: 698) |
| (Fragment 691) | |
| 5′-GGA GGG CGG C-3′ | (SEQ. ID NO: 699) |
| (Fragment 692) | |
| 5′-GA GGG CGG CAT GGC GGG CAC | (SEQ. ID NO: 700) |
| AGG CTG GGC-3′ (Fragment 693) | |
| 5′-GA GGG CGG CAT GGC GGG CAC | (SEQ. ID NO: 701) |
| AGG CTG GG-3′ (Fragment 694) | |
| 5′-GA GGG CGG CAT GGC GGG CAC | (SEQ. ID NO: 702) |
| AGG CTG G-3′ (Fragment 695) | |
| 5′-GA GGG CGG CAT GGC GGG CAC | (SEQ. ID NO: 703) |
| AGG CTG-3′ (Fragment 696) | |
| 5′-GA GGG CGG CAT GGC GGG CAC | (SEQ. ID NO: 704) |
| AGG CT-3′ (Fragment 697) | |
| 5′-GA GGG CGG CAT GGC GGG CAC | (SEQ. ID NO: 705) |
| AGG C-3′ (Fragment 698) | |
| 5′-GA GGG CGG CAT GGC GGG CAC | (SEQ. ID NO: 706) |
| AGG-3′ (Fragment 699) | |
| 5′-GA GGG CGG CAT GGC GGG CAC | (SEQ. ID NO: 707) |
| AG-3′ (Fragment 700) | |
| 5′-GA GGG CGG CAT GGC GGG CAC | (SEQ. ID NO: 708) |
| A-3′ (Fragment 701) | |
| 5′-GA GGG CGG CAT GGC GGG CAC-3′ | (SEQ. ID NO: 709) |
| (Fragment 702) | |
| 5′-GA GGG CGG CAT GGC GGG CA-3′ | (SEQ. ID NO: 710) |
| (Fragment 703) | |
| 5′-GA GGG CGG CAT GGC GGG C-3′ | (SEQ. ID NO: 711) |
| (Fragment 704) | |
| 5′-GA GGG CGG CAT GGC GGG-3′ | (SEQ. ID NO: 712) |
| (Fragment 705) | |
| 5′-GA GGG CGG CAT GGC GG-3′ | (SEQ. ID NO: 713) |
| (Fragment 706) | |
| 5′-GA GGG CGG CAT GGC G-3′ | (SEQ. ID NO: 714) |
| (Fragment 707) | |
| 5′-GA GGG CGG CAT GGC-3′ | (SEQ. ID NO: 715) |
| (Fragment 708) | |
| 5′-GA GGG CGG CAT GG-3′ | (SEQ. ID NO: 716) |
| (Fragment 709) | |
| 5′-GA GGG CGG CAT G-3′ | (SEQ. ID NO: 717) |
| (Fragment 710) | |
| 5′-GA GGG CGG CAT-3′ | (SEQ. ID NO: 718) |
| (Fragment 711) | |
| 5′-GA GGG CGG CA-3′ | (SEQ. ID NO: 719) |
| (Fragment 712) | |
| 5′-A GGG CGG CAT GGC GGG CAC AGG | (SEQ. ID NO: 720) |
| CTG GGC-3′ (Fragment 713) | |
| 5′-A GGG CGG CAT GGC GGG CAC AGG | (SEQ. ID NO: 721) |
| CTG GG-3′ (Fragment 714) | |
| 5′-A GGG CGG CAT GGC GGG CAC AGG | (SEQ. ID NO: 722) |
| CTG G-3′ (Fragment 715) | |
| 5′-A GGG CGG CAT GGC GGG CAC AGG | (SEQ. ID NO: 723) |
| CTG-3′ (Fragment 716) | |
| 5′-A GGG CGG CAT GGC GGG CAC AGG | (SEQ. ID NO: 724) |
| CT-3′ (Fragment 717) | |
| 5′-A GGG CGG CAT GGC GGG CAC AGG | (SEQ. ID NO: 725) |
| C-3′ (Fragment 718) | |
| 5′-A GGG CGG CAT GGC GGG CAC | (SEQ. ID NO: 726) |
| AGG-3′ (Fragment 719) | |
| 5′-A GGG CGG CAT GGC GGG CAC | (SEQ. ID NO: 727) |
| AG-3′ (Fragment 720) | |
| 5′-A GGG CGG CAT GGC GGG CAC | (SEQ. ID NO: 728) |
| A-3′ (Fragment 721) | |
| 5′-A GGG CGG CAT GGC GGG CAC-3′ | (SEQ. ID NO: 729) |
| (Fragment 722) | |
| 5′-A GGG CGG CAT GGC GGG CA-3′ | (SEQ. ID NO: 730) |
| (Fragment 723) | |
| 5′-A GGG CGG CAT GGC GGG C-3′ | (SEQ. ID NO: 731) |
| (Fragment 724) | |
| 5′-A GGG CGG CAT GGC GGG-3′ | (SEQ. ID NO: 732) |
| (Fragment 725) | |
| 5′-A GGG CGG CAT GGC GG-3′ | (SEQ. ID NO: 733) |
| (Fragment 726) | |
| 5′-A GGG CGG CAT GGC G-3′ | (SEQ. ID NO: 734) |
| (Fragment 727) | |
| 5′-A GGG CGG CAT GGC-3′ | (SEQ. ID NO: 735) |
| (Fragment 728) | |
| 5′-A GGG CGG CAT GG-3′ | (SEQ. ID NO: 736) |
| (Fragment 729) | |
| 5′-A GGG CGG CAT G-3′ | (SEQ. ID NO: 737) |
| (Fragment 730) | |
| 5′-A GGG CGG CAT-3′ | (SEQ. ID NO: 738) |
| (Fragment 731) | |
| 5′-GGG CGG CAT GGC GGG CAC AGG | (SEQ. ID NO: 739) |
| CTG GGC-3′ (Fragment 732) | |
| 5′-GGG CGG CAT GGC GGG CAC AGG | (SEQ. ID NO: 740) |
| CTG GG-3′ (Fragment 733) | |
| 5′-GGG CGG CAT GGC GGG CAC AGG | (SEQ. ID NO: 741) |
| CTG G-3′ (Fragment 734) | |
| 5′-GGG CGG CAT GGC GGG CAC AGG | (SEQ. ID NO: 742) |
| CTG-3′ (Fragment 735) | |
| 5′-GGG CGG CAT GGC GGG CAC AGG | (SEQ. ID NO: 743) |
| CT-3′ (Fragment 736) | |
| 5′-GGG CGG CAT GGC GGG CAC AGG | (SEQ. ID NO: 744) |
| C-3′ (Fragment 737) | |
| 5′-GGG CGG CAT GGC GGG CAC | (SEQ. ID NO: 745) |
| AGG-3′ (Fragment 738) | |
| 5′-GGG CGG CAT GGC GGG CAC AG-3′ | (SEQ. ID NO: 746) |
| (Fragment 739) | |
| 5′-GGG CGG CAT GGC GGG CAC A-3′ | (SEQ. ID NO: 747) |
| (Fragment 740) | |
| 5′-GGG CGG CAT GGC GGG CAC-3′ | (SEQ. ID NO: 748) |
| (Fragment 741) | |
| 5′-GGG CGG CAT GGC GGG CA-3′ | (SEQ. ID NO: 749) |
| (Fragment 742) | |
| 5′-GGG CGG CAT GGC GGG C-3′ | (SEQ. ID NO: 750) |
| (Fragment 743) | |
| 5′-GGG CGG CAT GGC GGG-3′ | (SEQ. ID NO: 751) |
| (Fragment 744) | |
| 5′-GGG CGG CAT GGC GG-3′ | (SEQ. ID NO: 752) |
| (Fragment 745) | |
| 5′-GGG CGG CAT GGC G-3′ | (SEQ. ID NO: 753) |
| (Fragment 746) | |
| 5′-GGG CGG CAT GGC-3′ | (SEQ. ID NO: 754) |
| (Fragment 747) | |
| 5′-GGG CGG CAT GG-3′ | (SEQ. ID NO: 755) |
| (Fragment 748) | |
| 5′-GGG CGG CAT G-3′ | (SEQ. ID NO: 756) |
| (Fragment 749) | |
| 5′-GG CGG CAT GGC GGG CAC AGG | (SEQ. ID NO: 757) |
| CTG GGC-3′ (Fragment 750) | |
| 5′-GG CGG CAT GGC GGG CAC AGG | (SEQ. ID NO: 758) |
| CTG GG-3′ (Fragment 751) | |
| 5′-GG CGG CAT GGC GGG CAC AGG | (SEQ. ID NO: 759) |
| CTG G-3′ (Fragment 752) | |
| 5′-GG CGG CAT GGC GGG CAC AGG | (SEQ. ID NO: 760) |
| CTG-3′ (Fragment 753) | |
| 5′-GG CGG CAT GGC GGG CAC AGG | (SEQ. ID NO: 761) |
| CT-3′ (Fragment 754) | |
| 5′-GG CGG CAT GGC GGG CAC AGG | (SEQ. ID NO: 762) |
| C-3′ (Fragment 755) | |
| 5′-GG CGG CAT GGC GGG CAC AGG-3′ | (SEQ. ID NO: 763) |
| (Fragment 756) | |
| 5′-GG CGG CAT GGC GGG CAC AG-3′ | (SEQ. ID NO: 764) |
| (Fragment 757) | |
| 5′-GG CGG CAT GGC GGG CAC A-3′ | (SEQ. ID NO: 765) |
| (Fragment 758) | |
| 5′-GG CGG CAT GGC GGG CAC-3′ | (SEQ. ID NO: 766) |
| (Fragment 759) | |
| 5′-GG CGG CAT GGC GGG CA-3′ | (SEQ. ID NO: 767) |
| (Fragment 760) | |
| 5′-GG CGG CAT GGC GGG C-3′ | (SEQ. ID NO: 768) |
| (Fragment 761) | |
| 5′-GG CGG CAT GGC GGG-3′ | (SEQ. ID NO: 769) |
| (Fragment 762) | |
| 5′-GG CGG CAT GGC GG-3′ | (SEQ. ID NO: 770) |
| (Fragment 763) | |
| 5′-GG CGG CAT GGC G-3′ | (SEQ. ID NO: 771) |
| (Fragment 764) | |
| 5′-GG CGG CAT GGC-3′ | (SEQ. ID NO: 772) |
| (Fragment 765) | |
| 5′-GG GGG CAT GG-3′ | (SEQ. ID NO: 773) |
| (Fragment 766) | |
| 5′-G CGG CAT GGC GGG CAC AGG CTG | (SEQ. ID NO: 774) |
| GGC-3′ (Fragment 767) | |
| 5′-G CGG CAT GGC GGG CAC AGG CTG | (SEQ. ID NO: 775) |
| GG-3′ (Fragment 768) | |
| 5′-G CGG CAT GGC GGG CAC AGG CTG | (SEQ. ID NO: 776) |
| G-3′ (Fragment 769) | |
| 5′-G CGG CAT GGC GGG CAC AGG | (SEQ. ID NO: 777) |
| CTG-3′ (Fragment 770) | |
| 5′-G CGG CAT GGC GGG CAC AGG | (SEQ. ID NO: 778) |
| CT-3′ (Fragment 771) | |
| 5′-G CGG CAT GGC GGG CAC AGG | (SEQ. ID NO: 779) |
| C-3′ (Fragment 772) | |
| 5′-G CGG CAT GGC GGG CAC AGG-3′ | (SEQ. ID NO: 780) |
| (Fragment 773) | |
| 5′-G CGG CAT GGC GGG CAC AG-3′ | (SEQ. ID NO: 781) |
| (Fragment 774) | |
| 5′-G CGG CAT GGC GGG CAC A-3′ | (SEQ. ID NO: 782) |
| (Fragment 775) | |
| 5′-G CGG CAT GGC GGG CAC-3′ | (SEQ. ID NO: 783) |
| (Fragment 776) | |
| 5′-G CGG CAT GGC GGG CA-3′ | (SEQ. ID NO: 784) |
| (Fragment 777) | |
| 5′-G CGG CAT GGC GGG C-3′ | (SEQ. ID NO: 785) |
| (Fragment 778) | |
| 5′-G CGG CAT GGC GGG-3′ | (SEQ. ID NO: 786) |
| (Fragment 779) | |
| 5′-G CGG CAT GGC GG-3′ | (SEQ. ID NO: 787) |
| (Fragment 780) | |
| 5′-G CGG CAT GGC G-3′ | (SEQ. ID NO: 788) |
| (Fragment 781) | |
| 5′-G CGG CAT GGC-3′ | (SEQ. ID NO: 789) |
| (Fragment 782) | |
| 5′-CGG CAT GGC GGG CAC AGG CTG | (SEQ. ID NO: 790) |
| GGC-3′ (Fragment 783) | |
| 5′-CGG CAT GGC GGG CAC AGG CTG | (SEQ. ID NO: 791) |
| GG-3′ (Fragment 784) | |
| 5′-CGG CAT GGC GGG CAC AGG CTG | (SEQ. ID NO: 792) |
| G-3′ (Fragment 785) | |
| 5′-CGG CAT GGC GGG CAC AGG | (SEQ. ID NO: 793) |
| CTG-3′ (Fragment 786) | |
| 5′-CGG CAT GGC GGG CAC AGG CT-3′ | (SEQ. ID NO: 794) |
| (Fragment 787) | |
| 5′-CGG CAT GGC GGG CAC AGG C-3′ | (SEQ. ID NO: 795) |
| (Fragment 788) | |
| 5′-CGG CAT GGC GGG CAC AGG-3′ | (SEQ. ID NO: 796) |
| (Fragment 789) | |
| 5′-CGG CAT GGC GGG CAC AG-3′ | (SEQ. ID NO: 797) |
| (Fragment 790) | |
| 5′-CGG CAT GGC GGG CAC A-3′ | (SEQ. ID NO: 798) |
| (Fragment 791) | |
| 5′-CGG CAT GGC GGG CAC-3′ | (SEQ. ID NO: 799) |
| (Fragment 792) | |
| 5′-CGG CAT GGC GGG CA-3′ | (SEQ. ID NO: 800) |
| (Fragment 793) | |
| 5′-CGG CAT GGC GGG C-3′ | (SEQ. ID NO: 801) |
| (Fragment 794) | |
| 5′-CGG CAT GGC GGG-3′ | (SEQ. ID NO: 802) |
| (Fragment 795) | |
| 5′-CGG CAT GGC GG-3′ | (SEQ. ID NO: 803) |
| (Fragment 796) | |
| 5′-CGG CAT GGC G-3′ | (SEQ. ID NO: 804) |
| (Fragment 797) | |
| 5′-GG CAT GGC GGG CAC AGG CTG | (SEQ. ID NO: 805) |
| GGG-3′ (Fragment 798) | |
| 5′-GG CAT GGC GGG CAC AGG CTG | (SEQ. ID NO: 806) |
| GG-3′ (Fragment 799) | |
| 5′-GG CAT GGC GGG CAC AGG CTG | (SEQ. ID NO: 807) |
| G-3′ (Fragment 800) | |
| 5′-GG CAT GGC GGG CAC AGG CTG-3′ | (SEQ. ID NO: 808) |
| (Fragment 801) | |
| 5′-GG CAT GGC GGG CAC AGG CT-3′ | (SEQ. ID NO: 809) |
| (Fragment 802) | |
| 5′-GG CAT GGC GGG CAC AGG C-3′ | (SEQ. ID NO: 810) |
| (Fragment 803) | |
| 5′-GG CAT GGG GGG CAC AGG-3′ | (SEQ. ID NO: 811) |
| (Fragment 804) | |
| 5′-GG CAT GGC GGG CAC AG-3′ | (SEQ. ID NO: 812) |
| (Fragment 805) | |
| 5′-GG CAT GGC GGG CAC A-3′ | (SEQ. ID NO: 813) |
| (Fragment 806) | |
| 5′-GG CAT GGG GGG CAC-3′ | (SEQ. ID NO: 814) |
| (Fragment 807) | |
| 5′-GG CAT GGG GGG CA-3′ | (SEQ. ID NO: 815) |
| (Fragment 808) | |
| 5′-GG CAT GGG GGG C-3′ | (SEQ. ID NO: 816) |
| (Fragment 809) | |
| 5′-GG CAT GGG GGG-3′ | (SEQ. ID NO: 817) |
| (Fragment 810) | |
| 5′-GG CAT GGG GG-3′ | (SEQ. ID NO: 818) |
| (Fragment 811) | |
| 5′-G CAT GGC GGG CAC AGG CTG | (SEQ. ID NO: 819) |
| GGC-3′ (Fragment 812) | |
| 5′-G CAT GGC GGG CAC AGG CTG | (SEQ. ID NO: 820) |
| GG-3′ (Fragment 813) | |
| 5′-G CAT GGC GGG CAC AGG CTG | (SEQ. ID NO: 821) |
| G-3′ (Fragment 814) | |
| 5′-G CAT GGC GGG CAC AGG CTG-3′ | (SEQ. ID NO: 822) |
| (Fragment 815) | |
| 5′-G CAT GGG GGG CAC AGG CT-3′ | (SEQ. ID NO: 823) |
| (Fragment 816) | |
| 5′-G CAT GGG GGG CAC AGG C-3′ | (SEQ. ID NO: 824) |
| (Fragment 817) | |
| 5′-G CAT GGG GGG CAC AGG-3′ | (SEQ. ID NO: 825) |
| (Fragment 818) | |
| 5′-G CAT GGC GGG CAC AG-3′ | (SEQ. ID NO: 826) |
| (Fragment 819) | |
| 5′-G CAT GGC GGG CAC A-3′ | (SEQ. ID NO: 827) |
| (Fragment 820) | |
| 5′-G CAT GGC GGG CAC-3′ | (SEQ. ID NO: 828) |
| (Fragment 821) | |
| 5′-G CAT GGG GGG CA-3′ | (SEQ. ID NO: 829) |
| (Fragment 822) | |
| 5′-G CAT GGC GGG C-3′ | (SEQ. ID NO: 830) |
| (Fragment 823) | |
| 5′-G CAT GGC GGG-3′ | (SEQ. ID NO: 831) |
| (Fragment 824) | |
| 5′-CAT GGC GGG CAC AGG CTG | (SEQ. ID NO: 832) |
| GGG-3′ (Fragment 825) | |
| 5′-CAT GGC GGG CAC AGG CTG GG-3′ | (SEQ. ID NO: 833) |
| (Fragment 826) | |
| 5′-CAT GGC GGG CAC AGG CTG G-3′ | (SEQ. ID NO: 834) |
| (Fragment 827) | |
| 5′-CAT GGC GGG CAC AGG CTG-3′ | (SEQ. ID NO: 835) |
| (Fragment 828) | |
| 5′-CAT GGC GGG CAC AGG CT-3′ | (SEQ. ID NO: 836) |
| (Fragment 829) | |
| 5′-CAT GGC GGG CAC AGG C-3′ | (SEQ. ID NO: 837) |
| (Fragment 830) | |
| 5′-GAT GGC GGG CAC AGG-3′ | (SEQ. ID NO: 838) |
| (Fragment 831) | |
| 5′-CAT GGC GGG CAC AG-3′ | (SEQ. ID NO: 839) |
| (Fragment 832) | |
| 5′-CAT GGC GGG CAC A-3′ | (SEQ. ID NO: 840) |
| (Fragment 833) | |
| 5′-CAT GGC GGG CAC-3′ | (SEQ. ID NO: 841) |
| (Fragment 834) | |
| 5′-CAT GGC GGG CA-3′ | (SEQ. ID NO: 842) |
| (Fragment 835) | |
| 5′-CAT GGC GGG C-3′ | (SEQ. ID NO: 843) |
| (Fragment 836) | |
| 5′-AT GGC GGG CAC AGG CTG GGC-3′ | (SEQ. ID NO: 844) |
| (Fragment 837) | |
| 5′-AT GGC GGG CAC AGG CTG GG-3′ | (SEQ. ID NO: 845) |
| (Fragment 838) | |
| 5′-AT GGC GGG CAC AGG CTG G-3′ | (SEQ. ID NO: 846) |
| (Fragment 839) | |
| 5′-AT GGC GGG CAC AGG CTG-3′ | (SEQ. ID NO: 847) |
| (Fragment 840) | |
| 5′-AT GGC GGG CAC AGG CT-3′ | (SEQ. ID NO: 848) |
| (Fragment 841) | |
| 5′-AT GGC GGG CAC AGG C-3′ | (SEQ. ID NO: 849) |
| (Fragment 842) | |
| 5′-AT GGC GGG CAC AGG-3′ | (SEQ. ID NO: 850) |
| (Fragment 843) | |
| 5′-AT GGC GGG CAC AG-3′ | (SEQ. ID NO: 851) |
| (Fragment 844) | |
| 5′-AT GGC GGG CAC A-3′ | (SEQ. ID NO: 852) |
| (Fragment 845) | |
| 5′-AT GGC GGG CAC-3′ | (SEQ. ID NO: 853) |
| (Fragment 846) | |
| 5′-AT GGC GGG CA-3′ | (SEQ. ID NO: 854) |
| (Fragment 847) | |
| 5′-T GGC GGG CAC AGG GTG GGC-3′ | (SEQ. ID NO: 855) |
| (Fragment 848) | |
| 5′-T GGC GGG CAC AGG CTG GG-3′ | (SEQ. ID NO: 856) |
| (Fragment 849) | |
| 5′-T GGC GGG CAC AGG CTG G-3′ | (SEQ. ID NO: 857) |
| (Fragment 850) | |
| 5′-T GGC GGG CAC AGG CTG-3′ | (SEQ. ID NO: 858) |
| (Fragment 851) | |
| 5′-T GGC GGG CAC AGG CT-3′ | (SEQ. ID NO: 859) |
| (Fragment 852) | |
| 5′-T GGC GGG CAC AGG C-3′ | (SEQ. ID NO: 860) |
| (Fragment 853) | |
| 5′-T GGC GGG CAC AGG-3′ | (SEQ. ID NO: 861) |
| (Fragment 854) | |
| 5′-T GGC GGG CAC AG-3′ | (SEQ. ID NO: 862) |
| (Fragment 855) | |
| 5′-T GGC GGG CAC A-3′ | (SEQ. ID NO: 863) |
| (Fragment 856) | |
| 5′-T GGC GGG CAC-3′ | (SEQ. ID NO: 864) |
| (Fragment 857) | |
| 5′-GGC GGG CAC AGG CTG GGC-3′ | (SEQ. ID NO: 865) |
| (Fragment 858) | |
| 5′-GGC GGG CAC AGG CTG GG-3′ | (SEQ. ID NO: 866) |
| (Fragment 859) | |
| 5′-GGC GGG CAC AGG CTG G-3′ | (SEQ. ID NO: 867) |
| (Fragment 860) | |
| 5′-GGC GGG CAC AGG CTG-3′ | (SEQ. ID NO: 868) |
| (Fragment 861) | |
| 5′-GGC GGG CAC AGG CT-3′ | (SEQ. ID NO: 869) |
| (Fragment 862) | |
| 5′-GGC GGG CAC AGG C-3′ | (SEQ. ID NO: 870) |
| (Fragment 863) | |
| 5′-GGC GGG CAC AGG-3′ | (SEQ. ID NO: 871) |
| (Fragment 864) | |
| 5′-GGC GGG CAC AG-3′ | (SEQ. ID NO: 872) |
| (Fragment 865) | |
| 5′-GGC GGG CAC A-3′ | (SEQ. ID NO: 873) |
| (Fragment 866) | |
| 5′-GC GGG CAC AGG CTG GGC-3′ | (SEQ. ID NO: 874) |
| (Fragment 867) | |
| 5′-GC GGG CAC AGG CTG GG-3′ | (SEQ. ID NO: 875) |
| (Fragment 868) | |
| 5′-GC GGG CAC AGG CTG G-3′ | (SEQ. ID NO: 876) |
| (Fragment 869) | |
| 5′-GC GGG CAC AGG CTG-3′ | (SEQ. ID NO: 877) |
| (Fragment 870) | |
| 5′-GC GGG CAC AGG CT-3′ | (SEQ. ID NO: 878) |
| (Fragment 871) | |
| 5′-GC GGG CAC AGG G-3′ | (SEQ. ID NO: 879) |
| (Fragment 872) | |
| 5′-GC GGG CAC AGG-3′ | (SEQ. ID NO: 880) |
| (Fragment 873) | |
| 5′-GC GGG CAC AG-3′ | (SEQ. ID NO: 881) |
| (Fragment 874) | |
| 5′-C GGG CAC AGG CTG GGC-3′ | (SEQ. ID NO: 882) |
| (Fragment 875) | |
| 5′-C GGG CAC AGG CTG GG-3′ | (SEQ. ID NO: 883) |
| (Fragment 876) | |
| 5′-C GGG CAC AGG CTG G-3′ | (SEQ. ID NO: 884) |
| (Fragment 877) | |
| 5′-C GGG CAC AGG CTG-3′ | (SEQ. ID NO: 885) |
| (Fragment 878) | |
| 5′-C GGG CAC AGG CT-3′ | (SEQ. ID NO: 886) |
| (Fragment 879) | |
| 5′-C GGG CAC AGG G-3′ | (SEQ. ID NO: 887) |
| (Fragment 880) | |
| 5′-C GGG CAC AGG-3′ | (SEQ. ID NO: 888) |
| (Fragment 881) | |
| 5′-GGG CAC AGG CTG GGC-3′ | (SEQ. ID NO: 889) |
| (Fragment 882) | |
| 5′-GGG CAC AGG CTG GG-3′ | (SEQ. ID NO: 890) |
| (Fragment 883) | |
| 5′-GGG CAC AGG CTG G-3′ | (SEQ. ID NO: 891) |
| (Fragment 884) | |
| 5′-GGG CAC AGG CTG-3′ | (SEQ. ID NO: 892) |
| (Fragment 885) | |
| 5′-GGG CAC AGG CT-3′ | (SEQ. ID NO: 893) |
| (Fragment 886) | |
| 5′-GGG CAC AGG G-3′ | (SEQ. ID NO: 894) |
| (Fragment 887) | |
| 5′-GC CAC AGG CTG GGG-3′ | (SEQ. ID NO: 895) |
| (Fragment 888) | |
| 5′-GC CAC AGG CTG GG-3′ | (SEQ. ID NO: 896) |
| (Fragment 889) | |
| 5′-GC CAC AGG CTG G-3′ | (SEQ. ID NO: 897) |
| (Fragment 890) | |
| 5′-GC CAC AGG CTG-3′ | (SEQ. ID NO: 898) |
| (Fragment 891) | |
| 5′-GC CAC AGG CT-3′ | (SEQ. ID NO: 899) |
| (Fragment 892) | |
| 5′-G CAC AGG CTG GGC-3′ | (SEQ. ID NO: 900) |
| (Fragment 893) | |
| 5′-G CAC AGG CTG GG-3′ | (SEQ. ID NO: 901) |
| (Fragment 894) | |
| 5′-G CAC AGG CTG G-3′ | (SEQ. ID NO: 902) |
| (Fragment 895) | |
| 5′-G CAC AGG CTG-3′ | (SEQ. ID NO: 903) |
| (Fragment 896) | |
| 5′-CAC AGG CTG GGG-3′ | (SEQ. ID NO: 904) |
| (Fragment 897) | |
| 5′-CAC AGG CTG GG-3′ | (SEQ. ID NO: 905) |
| (Fragment 898) | |
| 5′-CAC AGG CTG G-3′ | (SEQ. ID NO: 906) |
| (Fragment 899) | |
| 5′-AC AGG CTG GGC-3′ | (SEQ. ID NO: 907) |
| (Fragment 900) | |
| 5′-AC AGG CTG GG-3′ | (SEQ. ID NO: 908) |
| (Fragment 901) | |
| 5′-C AGG CTG GGC-3′ | (SEQ. ID NO: 909) |
| (Fragment 902) | |
| 5′-GGC GGC CTG GAA AGC TGA GAT | (SEQ. ID NO: 910) |
| GGA GGG CGG CAT GGC GGG CAC AGG | |
| CTG GGC-3′ (Fragment 903) | |
| 5′-GC GGC CTG GAA AGC TGA GAT | (SEQ. ID NO: 911) |
| GGA GGG CGG CAT GGC GGG CAC AGG | |
| CTG GGC-3′ (Fragment 904) | |
| 5′-C GGC CTG GAA AGC TGA GAT GGA | (SEQ. ID NO: 912) |
| GGG CGG CAT GGC GGG CAC AGG CTG | |
| GGC-3′ (Fragment 905) | |
| 5′-GGC CTG GAA AGC TGA GAT GGA | (SEQ. ID NO: 913) |
| GGG CGG CAT GGC GGG CAC AGG CTG | |
| GGC-3′ (Fragment 906) | |
| 5′-GC CTG GAA AGC TGA GAT GGA | (SEQ. ID NO: 914) |
| GGG CGG CAT GGC GGG CAC AGG CTG | |
| GGC-3′ (Fragment 907) | |
| 5′-C CTG GAA AGC TGA GAT GGA GGG | (SEQ. ID NO: 915) |
| CGG CAT GGC GGG CAC AGG CTG | |
| GGC-3′ (Fragment 908) | |
| 5′-CTG GAA AGC TGA GAT GGA GGG | (SEQ. ID NO: 916) |
| CGG CAT GGC GGG CAC AGG CTG | |
| GGC-3′ (Fragment 909) | |
| 5′-TG GAA AGC TGA GAT GGA GGG | (SEQ. ID NO: 917) |
| CGG CAT GGC GGG CAC AGG CTG | |
| GGC-3′ (Fragment 910) | |
| 5′-G GAA AGC TGA GAT GGA GGG CGG | (SEQ. ID NO: 918) |
| CAT GGC GGG CAC AGG CTG GGC-3′ | |
| (Fragment 911) | |
| 5′-GAA AGC TGA GAT GGA GGG CGG | (SEQ. ID NO: 919) |
| CAT GGC GGG CAC AGG CTG GGC-3′ | |
| (Fragment 912) | |
| 5′-AA AGC TGA GAT GGA GGG CGG | (SEQ. ID NO: 920) |
| CAT GGC GGG CAC AGG CTG GGC-3′ | |
| (Fragment 913) | |
| 5′-A AGC TGA GAT GGA GGG CGG CAT | (SEQ. ID NO: 921) |
| GGC GGG CAC AGG CTG GGC-3′ | |
| (Fragment 914) | |
| 5′-AGC TGA GAT GGA GGG CGG CAT | (SEQ. ID NO: 922) |
| GGC GGG CAC AGG CTG GGC-3′ | |
| (Fragment 915) | |
| 5′-GC TGA GAT GGA GGG CGG CAT | (SEQ. ID NO: 923) |
| GGC GGG CAC AGG CTG GGC-3′ | |
| (Fragment 916) | |
| 5′-C TGA GAT GGA GGG CGG CAT GGC | (SEQ. ID NO: 924) |
| GGG CAC AGG CTG GGC-3′ | |
| (Fragment 917) | |
| 5′-TGA GAT GGA GGG CGG CAT GGC | (SEQ. ID NO: 925) |
| GGG CAC AGG CTG GGC-3′ | |
| (Fragment 918) | |
| 5′-GA GAT GGA GGG CGG CAT GGC | (SEQ. ID NO: 926) |
| GGG CAC AGG CTG GGC-3′ | |
| (Fragment 919) | |
| 5′-A GAT GGA GGG CGG CAT GGC GGG | (SEQ. ID NO: 927) |
| CAC AGG CTG GGC-3′ | |
| (Fragment 920) | |
| 5′-GAT GGA GGG CGG CAT GGC GGG | (SEQ. ID NO: 928) |
| CAC AGG CTG GGC-3′ | |
| (Fragment 921) | |
| 5′-AT GGA GGG CGG CAT GGC GGG | (SEQ. ID NO: 929) |
| CAC AGG CTG GGC-3′ | |
| (Fragment 922) | |
| 5′-T GGA GGG CGG CAT GGC GGG CAC | (SEQ. ID NO: 930) |
| AGG CTG GGC-3′ (Fragment 923) | |
| 5′-GGA GGG CGG CAT GGC GGG CAC | (SEQ. ID NO: 931) |
| AGG CTG GGC-3′ (Fragment 924) | |
| 5′-GA GGG CGG CAT GGC GGG CAC | (SEQ. ID NO: 932) |
| AGG CTG GGC-3′ (Fragment 925) | |
| 5′-A GGG GGG CAT GGC GGG CAC AGG | (SEQ. ID NO: 933) |
| CTG GGG-3′ (Fragment 926) | |
| 5′-GGG CGG CAT GGG GGG CAC AGG | (SEQ. ID NO: 934) |
| CTG GGC-3′ (Fragment 927) | |
| 5′-GG CGG CAT GGG GGG CAC AGG | (SEQ. ID NO: 935) |
| CTG GGG-3′ (Fragment 928) | |
| 5′-G CGG CAT GGC GGG CAC AGG CTG | (SEQ. ID NO: 936) |
| GGC-3′ (Fragment 929) | |
| 5′-CGG CAT GGC GGG CAC AGG CTG | (SEQ. ID NO: 937) |
| GGC-3′ (Fragment 930) | |
| 5′-GG CAT GGC GGG CAC AGG CTG | (SEQ. ID NO: 938) |
| GGG-3′ (Fragment 931) | |
| 5′-G CAT GGG GGG CAC AGG CTG | (SEQ. ID NO: 939) |
| GGC-3′ (Fragment 932) | |
| 5′-CAT GGG GGG CAC AGG CTG | (SEQ. ID NO: 940) |
| GGC-3′ (Fragment 933) | |
| 5′-AT GGC GGG CAC AGG CTG GGC-3′ | (SEQ. ID NO: 941) |
| (Fragment 934) | |
| 5′-T GGC GGG CAC AGG CTG GGG-3′ | (SEQ. ID NO: 942) |
| (Fragment 935) | |
| 5′-GGC GGG CAC AGG CTG GGC-3′ | (SEQ. ID NO: 943) |
| (Fragment 936) | |
| 5′-GC GGG CAC AGG CTG GGC-3′ | (SEQ. ID NO: 944) |
| (Fragment 937) | |
| 5′-C GGG CAC AGG CTG GGC-3′ | (SEQ. ID NO: 945) |
| (Fragment 938) | |
| 5′-GGG CAC AGG CTG GGC-3′ | (SEQ. ID NO: 946) |
| (Fragment 939) | |
| 5′-GG CAC AGG CTG GGG-3′ | (SEQ. ID NO: 947) |
| (Fragment 940) | |
| 5′-G CAC AGG CTG GGC-3′ | (SEQ. ID NO: 948) |
| (Fragment 941) | |
| 5′-CAC AGG CTG GGC-3′ | (SEQ. ID NO: 949) |
| (Fragment 942) | |
| 5′-AG AGG CTG GGC-3′ | (SEQ. ID NO: 950) |
| (Fragment 943) | |
| 5′-C AGG CTG GGC-3′ | (SEQ. ID NO: 951) |
| (Fragment 944) | |
| 5′-AGG CTG GGC-3′ | (SEQ. ID NO: 952) |
| (Fragment 945) | |

Other adenosine fragments, for example those with low adenosine content or lacking adenosine altogether, are also suitable and in some cases even preferred, for use with the invention. The following sequences, their fragments and combinations, are one particularly preferred group of anti-sense oligos.

| | | |
|---|---|---|
| TTT TCC TTC CTT TGT CTC TCT TC | (FRAG 946) | (SEQ. ID NO: 953) |
| GCT CCC GGC TGC CTG | (FRAG 947) | (SEQ. ID NO: 954) |
| CTC GGC CGT GCG GCT CTG TCG CTG CCG GT | (FRAG 948) | (SEQ. ID NO: 955) |
| CCG CCG CCC TCC GGG GGG TC | (FRAG 949) | (SEQ. ID NO: 956) |
| TGC TGC CGT TGG CTG CCC | (FRAG 950) | (SEQ. ID NO: 957) |
| CTT CTG CGG GTC GCC GG | (FRAG 951) | (SEQ. ID NO: 958) |
| TGC TGG GCT TGT GGC | (FRAG 952) | (SEQ. ID NO: 959) |
| GGC CTC TCT TCT GGG | (FRAG 953) | (SEQ. ID NO: 960) |
| CCT GGT CCC TCC GT | (FRAG 954) | (SEQ. ID NO: 961) |
| GGT GGC TCC TCT GC | (FRAG 955) | (SEQ. ID NO: 962) |
| GCT TGG TCC TGG GGC TGC | (FRAG 956) | (SEQ. ID NO: 963) |
| TGC TCT CCT CTC CTT | (FRAG 957) | (SEQ. ID NO: 964) |

In another embodiment of this invention, the oligos are anti-sense to an adenosine A₂ₐ receptor, and must either be “up-regulated”, or if they have some adenosine A₁ activity they are treated as the other anti-sense oligos. The following sequences are preferred examples of anti-sense oligos associated with the human adenosine A₂ₐ receptor. Another preferred group is composed of fragments of these sequences and combinations thereof as well as mixtures. Also preferred are these sequences, fragments and their combinations where one or more adenosines are substituted by a universal base or an adenosine analogue which either is not an agonist or a ligand for the adenosine A₁ receptor, or which acts as an antagonist of the A₁ receptor, such as, for example, theophylline or enprophylline.

| | | |
|---|---|---|
| 5′-TGC TTT TCT TTT CTG GGC CTC -3′ | (FRAG 958) | (SEQ. ID NO: 965) |
| 5′-TGT GGT CTG TTT TTT TCT G-3′ | (FRAG 959) | (SEQ. ID NO: 966) |
| 5′-GCC CTG CTG GGG CGC TCT CC-3′ | (FRAG 960) | (SEQ. ID NO: 967) |
| 5′-GCC GCC CGC CTG GCT CCC-3′ | (FRAG 961) | (SEQ. ID NO: 968) |
| 5′-GGB GCC CBT GBT GGG CBT GCC-3′ | (FRAG 962) | (SEQ. ID NO: 969) |
| 5′-GTG GTT CTT GCC CTC CTT TGG CTG-3′ | (FRAG 963) | (SEQ. ID NO: 970) |
| 5′-CCG TGC CCG CTC CCC GGC-3′ | (FRAG 964) | (SEQ. ID NO: 971) |
| 5′-CTC CTG GCG GGT GGC CGT TG-3′ | (FRAG 965) | (SEQ. ID NO: 972) |
| 5′-GGC CCG TGT TCC CCT GGG-3′ | (FRAG 966) | (SEQ. ID NO: 973) |
| 5′-GCC TGG G GC TCC CTT CTC TC-3′ | (FRAG 967) | (SEQ. ID NO: 974) |
| 5′-GCC CTT CTT GCT GGG CCT C-3′ | (FRAG 968) | (SEQ. ID NO: 975) |
| 5′-TGC TGC TGC TGG TGC TGT GGC CCC C-3′ | (FRAG 969) | (SEQ. ID NO: 976) |
| GTACACCGAGGAGCCCATGATGGGCATGCCACAGACGACAGGC | (FRAG 970) | (SEQ. ID NO: 977) |
| GTBCBCCGBGGBGCCCBTGBTGGGCBTGCCBCBGBCGBCBGGC | (FRAG 971) | (SEQ. ID NO: 978) |

In another embodiment, the anti-sense oligo of the invention may be a sequence which is anti-sense to the adenosine A_{2b} receptor. By means of example, the following sequencess associated with the human receptor are provided. These sequences as well as their fragments and combinations, desadenosine fragments and those where one or more A are substituted with a universal base or adenosine analogue as described above are preferred.

| | | |
|---|---|---|
| 5′-GGC GCC GTG CCG CGT CTT GGT GGC GGC GG-3′ | (FRAG 972) | (SEQ. ID NO: 979) |
| 5′-GTT CGC GCC CGC GCG GGG CCC CTC CGG TCC-3′ | (FRAG 973) | (SEQ. ID NO: 980) |
| 5′-TTG GCC CGC GCG CCC GCC CGT CTC GGG CTG GGC GG-3 | (FRAG 974) | (SEQ. ID NO: 981) |
| 5′-CGG GTC GGG GCC CCC CGC GGC C-3′ | (FRAG 975) | (SEQ. ID NO: 982) |
| 5′-GCC TCG GGG CTG GGG CGC TGG TGG CCG GG-3′ | (FRAG 976) | (SEQ. ID NO: 983) |
| 5′-CCG CGC CTC CGC CTG CCG CTT CTG-3′ | (FRAG 977) | (SEQ. ID NO: 984) |
| 5′-GCT GGG CCC CGG GCG CCC CCT-3′ | (FRAG 978) | (SEQ. ID NO: 985) |
| 5′-CCC CTC TTG CTC GGG TCC CCG TG-3′ | (FRAG 979) | (SEQ. ID NO: 986) |
| ACAGCGCGTCCTGTGTCTCCAGCAGCATGGCCGGGCCAGCTGGGCCCC | (FRAG 980) | (SEQ. ID NO: 987) |
| BCBGCGCGTCCTGTGTCTCCBGCBGCBTGGCCGGGCCBGCTGGGCCCC | (FRAG 981) | (SEQ. ID NO: 988) |

In still another embodiment, the oligo of this invention may be anti-sense to any fragment of the adenosine A₃ receptor gene or mRNA, including overlapping regions with the flanking regions or introns. The following are examples of these fragments associated with the human receptor. These are preferred sequences. Also preferred are their fragments and combinations, as well as desadenosine fragments and those where one or more A are substituted by a universal base or A analogue as described above.

| | | |
|---|---|---|
| ACA GAG CA TGC TGT TGT TGG GCA TCT TGC CTT CCC AGG G | (FRAG 982) | (SEQ. ID NO: 989) |
| BCB GBG CB TGC TGT TGT TGG GCB TCT TGC CTT CCC BGG G | (FRAG 983) | (SEQ. ID NO: 990) |
| CCC TTT TCT GGT GGG GTG | (FRAG 984) | (SEQ. ID NO: 991) |
| GTG CTG TTG TTG GGC | (FRAG 985) | (SEQ. ID NO: 992) |
| TTT CTT CTG TTT CC | (FRAG 986) | (SEQ. ID NO: 993) |
| CCC TTT TCT GGT GGG GTG | (FRAG 987) | (SEQ. ID NO: 994) |
| GTG CTG TTG TTG GGC | (FRAG 988) | (SEQ. ID NO: 995) |
| TTT CTT CTG TTC CC | (FRAG 989) | (SEQ. ID NO: 996) |

In the anti-sense oligonucleotides of the present invention, exemplified by the preceding sequences, a number of adenosine bases may be replaced with an appropriate “spacer” or universal base (e.g., 1-[β-D-2′-deoxyribofuranosyl]-5-nitroindole], or with an adenosine agonist or antagonist that does not stimulate adenosine A₁, A_{2b} or A₃ receptors, but which may stimulate adenosine A₂ₐ receptors. In this manner, a specific adenosine receptor gene may be targeted to obtain one or more anti-sense oligonucleotide(s) (oligos) that selectively bind(s) to the corresponding mRNA, and then, if necessary, their content of adenosine may be reduced by substituting one or more universal bases or adenosine analogues incapable of activating adenosine A₁, A_{2b} or A₃ receptors or which activate the adenosine A₂ₐ receptor. Thus, in addition to “down-regulating” specific adenosine receptor genes, the present oligos have an increased effect when administered by either selection of genes, RNA and flanking regions that are devoid, or have a low A content, or alternatively one or more of the adenosine(s) present in the oligonucleotide(s) are substituted with other nucleotide bases, so called universal bases, which bind to thymidine (T) but lack the ability to activate adenosine receptors and otherwise may not activate adenosine receptors. Given that adenosine (A) is a nucleotide base complementary to thymidine (T), when a T appears in the RNA, the anti-sense oligo will have an A at the same position.

The method of the present invention may be used to treat ailments associated with or causing bronchoconstriction, allegy(ies) and/or inflammation associated with a ny of the diseases and conditions described above in a subject, regardless of its cause. The anti-sense agent(s) of the invention have preferably a low (or reduced) A content to prevent its liberation upon in vivo degradation of the agent(s), preferably up to about 15%, more preferably up to about 10%, still more preferably up to about 5%, and even more preferred being devoid of A (“desade nosine oligos”).

The oligos of this invention may be obtained by first selecting fragments of a target nucleic acid having at least 4 contiguous nucleic acids selected from the group consisting of G and C, and then obtaining a first oligonucleotide 4 to 60 nucleotide long which comprises the selected fragment and has a C and G nucleic acid content of up to and including about 15%. The latter step may be conducted by obtaining a second oligonucleotide 4 to 60 nucleotide long comprising a sequence which is anti-sense to the selected fragment, the second oligonucleotide having an adenosine base content of up to and including about 15%. This method may also comprise, when the selected fragment comprises at least one thymidine base, substituting an adenosine base in the corresponding nucleotide of the anti-sense fragment with a universal base selected from the group consisting of heteroaromatic bases which bind to a thymidine base but have antagonist activity and less than about 0.3 of the adenosine base agonist activity at the adenosine A₁, A_{2b} and A₃ receptors, and heteroaromatic bases which have no activity or have an agonist activity at the adenosine A₂ₐ receptor. The analogue heteroaromatic bases may be selected from all pyrimidines and purines, which may be substituted by O, halo, NH₂, SH, SO; SO₂, SO₃, COOH and branched and fused primary and secondary amino, alkyl, alkenyl, alkynyl, cycloalkyl, heterocycloalkyl, aryl, heteroaryl, alkoxy, alkenoxy, acyl, cycloacyl, arylacyl, alkynoxy, cycloalkoxy, aroyl, arylthio, arylsulfoxyl, halocycloalkyl, alkylcycloalkyl, alkenylcycloalkyl, alkynylcycloalkyl, haloaryl, alkylaryl, alkenylaryl, alkynylaryl, arylalkyl, arylalkenyl, arylalkynyl, arylcycloalkyl, which may be further substituted by O, halo, NH₂, primary, secondary and tertiary amine, SH, SO, SO₂, SO₃, cycloalkyl, heterocycloalkyl and heteroaryl. The pyrimidines and purines may be substituted at all positions as is known in the art, but preferred are those which are substituted at positions 1, 2, 3, 4, 7 and/or 8. More preferred are pyrimidines and purines such as theophylline, caffeine, dyphylline, etophylline, acephylline piperazine, bamifylline, enprofylline and xantine having the chemical formula wherein R¹ and R² are independently H, alkyl, alkenyl or alkynyl and R³ is H, aryl, dicycloalkyl, dicycloalkenyl, dicycloalkynyl, cycloalkyl, cycloalkenyl, cycloalkynyl, O-cycloalkyl, O-cycloalkenyl, O-cycloalkynyl, NH₂-alkylamino-ketoxyalkyloxy-aryl, mono and dialkylaminoalkyl-N-alkylamino-SO₂ aryl, among others. However, other methods may also be employed. The inventor reduced the adenosine content of the anti-sense oligos corresponding to the thymidines (T) present in the target gene, RNA, flanking regions, and bridging sections to less than about 15%, or fully eliminated A from the oligonucleotide sequence as a means for preventing their breakdown products from freeing adenosine into the lung tissue environment and, thereby, aggravating the subject's ailment and/or countering the beneficial effect of the administered.

Also part of this invention are chemical analogues of the nucleic acids in which, for example, the phosphodiester bonds have been modified, e.g., to a methylphosphonate, a phosphotriester, a phosphorothioate, a phosphorodithioate, or a phosphoramidate, so as to render the nucleic acids more stable in vivo. The naturally occurring phosphodiester linkages in nucleic acids are susceptible to degradation by endogenously occurring cellular nucleases, while many analogous linkages are highly resistant to nuclease degradation. See Milligan et al., and Cohen, J. S., supra. The use of a “3′-end cap” strategy by which nuclease-resistant linkages are substituted for phosphodiester linkages at the 3′ end of the oligonucleotide protects oligonucleotides from degradation. See, Tidd, D. M. and Warenius, H. M., Br. J. Cancer 60, 343-350 (1989); Shaw, J. P. et al., Nucleic Acids Res. 19, 747-750 (1991). Phosphoramidate, phosphorothioate, and methylphosphonate linkages are suitable for use in this invention. In addition, extensive modification of the phosphodiester backbone has been shown to impart stability and may allow for enhanced affinity and increased cellular permeation of oligonucleotides. See Milligan, et al., supra. Many different chemical strategies have been employed to replace the entire phosphodiester backbone with novel linkages. Id. The analogues of the oligonucleotides of the invention include phosphorothioate, phosphorodithioate, phosphorotrithioate, methylphosphonate, phosphoramidate, boranophosphate, phosphotriester, formacetal, 2′-O-methyl, Thioformacetal such as 3′-thioformacetal and 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) and methyleneoxy (methylimino) (MOMI) linkages, among others. The oligonucleotides of the invention may also be modified by addition of a terminal 1,3-propanediol or a terminal dodecanol, among others, or they may be conjugated to a polyethylene glycol, cholesterol, cholesteryl, dehydroepiandrosterone, dehydroepiandrosterone sulfate, dehydroepiandrosterone sulfatide, ubiquinone, dolichol, poly L-lysine, sulfatidic acid and fatty acid, among others. The oligos of the invention may also be modified by 2′-O-methoxyethyl, C-5-propynyl pyrimidine, C-5 methyl cytidine, C-5 ethynyl pyrimidine, 2′-propoxy, C-18 amine, N3′-P5′ phosphoramidates, 3′-alkylamino, 2′-fluoro; 5-fluoro pyrimidine, 5-iodo pyrimidine, 5-bromo pyrimidine, 2′-borano, C-5 hexynyl pyrimidine, 2′-O-(2-methoxy)ethyl, 2′-O-aminopropyl, 5-(phenylethyl) and peptide nucleic acid interbase linkages. Phosphorothioate and methylphosphonate-modified oligonucleotides are particularly preferred because of their availability and suitability for automated oligonucleotide synthesis. Id. Anti-sense oligonucleotides containing modifications to the nucleotide base itself, e.g. a C-5 propyne, or to the sugar, e.g. a carbohydrate modification, are also aspects of the present invention.

Where appropriate, the antisense nucleotide may be administered in the form of their pharmaceutically acceptable salts or as a mixture.

Anti-sense oligonucleotides may be of any suitable length, e.g., from about 7 to 60 nucleotide in length, depending on the particular target being bound and their mode of delivery. Preferably the antisense oligonucleotide is directed to a gene or mRNA region containing a junction between intron and exon. Where the anti-sense oligonucleotide is directed to an intron/exon junction, it may either entirely overlie the junction or may be sufficiently close to the junction to inhibit the splicing out of the intervening exon during processing of precursor mRNA to mature mRNA, e.g., with the 3′ or 5′ terminus of the antisense oligonucleotide being positioned within about, for example, 10, 5, 3, or 2 nucleotide of the intron/exon junction. Also preferred are anti-sense oligonucleotides which overlap the initiation codon.

When practicing the present invention, the anti-sense oligonucleotides administered may be related in origin to the species to which it is administered. When treating humans, the anti-sense may be derived from human sequences. However, sequences obtained from one species are also suitable for administration to a second species.

The pharmaceutical compositions provided herein comprise nucleic acid(s) comprising the anti-sense oligonucleotide(s) described above and one or more surfactants. Suitable surfactants or surfactant components for enhancing the uptake of the anti-sense oligonucleotides of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamelar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomeric and polymeric, poly(vinyl amine) with dextran and/or alkanoyl side chains, Brij 35, Triton X-100 and synthetic surfactants ALEC, Exosurf, Survan and Atovaquone, among others. These surfactants may be useed either as single or part of a multiple component surfactant in a formulation, or as covalently bound additions to the 5′ and/or 3′ ends of the anti-sense oligonucleotides (oligos).

These compositions are administered in amounts effective to reduce the expression of an adenosine receptor, such as the adenosine A₁, A_{2b} or A₃ receptor by passing through a cell membrane and binding specifically with mRNA encoding an adenosine A₁, A_{2b} or A₃ receptor in the cell and prevent its translation. In addition, the present oligos may be targeted to the adenosine A₂ₐ receptor, as long as they have some anti-A₁, A_{2b} or A₃ receptor activity. Such compositions may contain a suitable pharmaceutically acceptable carrier, e.g., sterile pyrogen-free saline solution, and the like. The present pharmaceutical compositions may be formulated as topical and systemic formulations, in a variety of types, including oral, buccal, nasal, otical, rectal, inhalable, slow release, enteric coated, dermal, intradermal, injectable, and many more as is known in the art. The formulation of the invention may also comprise a hydrophobic carrier capable of passing through a cell membrane, e.g., in a liposome, with the liposomes carried in a pharmaceutically acceptable aqueous carrier. The oligonucleotides may also be coupled to a substance which inactivates mRNA, such as a ribozyme. The present compositions may be administered to a subject afflicted with a disease or condition associated with the stimulation of lung adenosine A₁, A₂ₐ, A_{2b} or A₃ receptors, such as any of the ones described above, in order to inhibit the activation of the adenosine receptors. The pharmaceutical formulation may also contain chimeric molecules comprising antisense oligonucleotides attached to molecules which are known to be internalized by cells either in a non-specific or in a tissue-specific manner. These oligonucleotide conjugates utilize cellular uptake pathways to increase the cellular concentrations of oligonucleotides. Examples of macromolecules used in this manner include transferrin, asialoglycoprotein, e.g. bound to oligonucleotides via polylysine, streptavidin, or other chemical linkages.

The anti-sense compound may be contained in the pharmaceutical formulation within a lipid particle or vesicle, such as a liposome or microcrystal. The lipid particles may be of any suitable structure, such as unilamellar or plurilamellar, so long as the antisense oligonucleotide is contained therein. Positively charged lipids such as N-[1-(2,3-dioleoyloxi)propyl]-N,N,N-trimethyl-ammoniumethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The composition of the invention may be administered by any means which transports the anti-sense nucleotide and the surfactant composition to the lung. The antisense compounds disclosed herein may be administered to the lungs of a patient by any suitable means, but are preferably administered by inhalation of an aerosol comprised of respirable particles which comprise the anti-sense compound. The respirable particles may be liquid or solid, and they may optionally contain other therapeutic or diagnostic ingredients as well as other typical ingredients for a particular formulation. Examples of other agents are analgesics such as acetominophen, anilerdine, aspirin, buprenorphine, butabital, butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti-anxiety agents associated with mental depression, such as Chlordiazepoxide, Amitriptyline, Loxapine Maprotiline and Perphenazine, among others. Anti-inflammatory agents such as non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Sedatives including Diphenhydramine, Hydroxyzine, Methotrimeprazine, Promethazine, Propofol, Melatonin, Trimeprazine, and the like. Sedatives and agents used for treatment of petit mal and tremors, among other conditions, such as Amitriptyline HCl; Chlordiazepoxide, Amobarbital; Secobarbital, Aprobarbital, Butabarbital, Ethchiorvynol, Glutethimide, L-Tryptophan, Mephobarbital, MethoHexital Na, Midazolam Hcl, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g. for treatment of severe head injury; orphan status, Warner Lambert), cytoprotective agents, and agents for the treatment of menopause, menopausal symptoms (treatment), e.g. Ergotamine, Belladonna Alkaloids and Phenobarbital, for the treatment of menopausal vasomotor symptoms, e.g. Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric treatments such as Tricyclic Antidepressants, including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Ssrotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications.

The composition of the present invention may be administered into the respiratory system as a formulation including particles of respirable size, e.g. particles of a size sufficiently small to pass through the nose, mouth and larynx upon inhalation and through the bronchi and alveoli of the lungs. In general, respirable particles range from about 0.5 to 10 microns in size. Particles of non-respirable size which are included in the aerosol tend to deposit in the throat and be swallowed, and the quantity of non-respirable particles in the aerosol is thus minimized. For nasal administration, a particle size in the range of 10-500 μm is preferred to ensure retention in the nasal cavity.

Liquid pharmaceutical compositions of active compound for producing an aerosol may be prepared by combining the antisense compound with a suitable vehicle, such as sterile pyrogen free water. Other therapeutic compounds may optionally be included.

Solid particulate compositions containing respirable dry particles of micronized antisense compound may be prepared by grinding dry antisense compound with a mortar and pestle, and then passing the micronized composition through a 400 mesh screen to break up or separate out large agglomerates. A solid particulate composition comprising of the antisense compound may optionally contain a dispersant which serves to facilitate the formation of an aerosol as well as other therapeutic compounds. A suitable dispersant is lactose, which may be blended with the antisense compound in any suitable ratio, e.g., a 1 to 1 ratio by weight.

The anti-sense compound may be administered in an anti-brochoconstriction, anti-allergy(ies) and/or anti-inflammatory effective amount, which amount depends upon the degree of disease being treated, the condition of the subject, the particular formulation, the route of administration, the timing of administration to a subject, etc.

In general, intracellular concentrations of the oligonucleotide of from 0.05 to 50 μM, or more particularly 0.2 to 5 μM, are desirable. For administration to a subject such as a human, a dosage of about 0.01, 0.1, or 1 mg/Kg up to about 50, 100, or 150 mg/Kg or more is typically employed. However, other doses are also contemplated in this patent. Depending on the solubility of the active compound in any particular formulation, the daily dose may be divided among one or several unit dose administrations.

The aerosols of liquid particles comprising the antisense compound may be produced by any suitable means, such as with a nebulizer. See, e.g., U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable formulations for use in nebulizers comprise the active ingredient in a liquid carrier in an amount of up to 40% w/w preferably less than 20% w/w of the formulation. The carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, anti-oxidants, flavorings, volatile oils, buffering agents and emulsifiers and other formulation surfactants.

The aerosols of solid particles comprising the active compound and surfactant may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder, e.g., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 150 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, emulsifiers and other formulation surfactants, such as oleic acid or sorbitan trioleate, anti-oxidants and suitable flavoring agents.

The aerosol, whether formed from solid or liquid particles, may be produced by the aerosol generator at a rate of from about 10 to 150 liters per minute, more preferably from about 30 to 150 liters per minute, and most preferably about 60 liters per minute. Aerosols containing greater amounts of medicament may be administered more rapidly.

The following examples are provided to illustrate the present invention, and should not be construed as limiting thereon. In these examples, μM means micromolar, mL means milliliters, μm means micrometers, mm means millimeters, cm means centimeters, ° C. means degrees Celsius, μg means micrograms, mg means milligrams, g means grams, kg means kilograms, M means molar, and h means hours.

### EXAMPLES

### Example 1

### Design and Synthesis of Anti-sense Oligonucleotides & Controls

The design of anti-sense oligonucleotides against the adenosine receptors is based on the primary and secondary structure of the target receptor mRNA. The anti-sense oligonucleotides are selected, and optimally modified, to target regions of mRNA which confer functional activity or stability to the mRNA and which preferably may overlap the initiation codon. For instance, regions that afford particularly strong binding, such as CG strings are preferred, i.e. runs of G and/or C, preferably at the 5′-end of the target region within the target gene or mRNA. However, other target sites within the molecule are suitable as well, particularly those which have low sequence overlapping with other gene sequences, thus increasing the specificity of the treatment.

Other oligonucleotides not totally complementary to the target mRNA, but containing identical nucleotide compositions on a w/w basis (controls), are included as controls in anti-sense experiments to demonstrate the specificity of the activity of the agents of this invention.

The primary and secondary structure of the human adenosine A₁ receptor mRNA was analyzed and used as described above to design anti-sense oligonucleotides, including the ones, whose sequences are provided. One anti-sense oligonucleotide (Oligo I) was synthesized as a phosphorothioate, designated HAdA1AS, and has the following sequence:

| | |
|---|---|
| 5′-GAT GGA GGG CGG CAT GGC GGG-3′ | (SEQ ID NO: 1) |

As a control, a mismatched phosphorothioate anti-sense nucleotide designated HAdA1MM was synthesized with the following sequence.

| | |
|---|---|
| 5′-GTA GCA GGC GGG GAT GGG GGC-3′ | (SEQ ID NO: 2) |

The oligonucleotides of SEQ. ID NOS:1 and 2 shown above have identical base contents and general sequence structures. Homology searches in GENBANK (release 85.0) and EMBL (release 40.0) indicated that the anti-sense oligonucleotide was specific for the human and rabbit adenosine A₁ receptor genes, and that the mis-matched control was not a candidate for hybridization with any known gene sequence.

In the same manner, the primary and secondary structure of the human adenosine A₃ receptor mRNA was analyzed and various oligos selected, and the following two synthesized as phosphorothioate anti-sense oligonucleotides. The first anti-sense oligonucleotide (HAdA3AS1) synthesized has the following sequence.

| | |
|---|---|
| 5′-GTT GTT GGG CAT CTT GCC-3′ | (SEQ ID NO: 3) |

As a control, a mis-matched phosphorothioate anti-sense oligonucleotide (HAdA3MM1) was synthesized, which has the following sequence.

| | |
|---|---|
| 5′-GTA CTT GCG GAT CTA GGC-3′ | (SEQ ID NO: 4) |

The second phosphorothioate anti-sense oligonucleotide (HadA3AS2) has the following sequence.

| | |
|---|---|
| 5′-GTG GGC CTA GCT CTC GCC-3′ | (SEQ ID NO: 5) |

As a control, its mis-matched oligonucleotide (HAdA3MM2) has the following sequence.

| | |
|---|---|
| 5′-GTC GGG GTA CCT GTC GGC-3′ | (SEQ ID NO:6) |

All phosphorothioate oligonucleotides were synthesized on an Applied Biosystems Model 396 Oligonucleotide Synthesizer, and purified using NENSORB chromatography (DuPont, MD).

### Example 2

### In Vitro Testing of A₁-Adenosine Receptor Anti-sense Oligonucleotides

The anti-sense oligonucleotide against the human A₁ receptor (SEQ ID NO:1) described above was tested for efficacy in an in vitro model utilizing lung adenocarcinoma cells HTB-54. HTB-54 lung adenocarcinoma cells were demonstrated to express the A₁ adenosine receptor using standard northern blotting procedures and receptor probes designed and synthesized in the laboratory.

HTB-54 human lung adenocarcinoma cells (10⁶/100 mm tissue culture dish) were exposed to 5.0 μM HAdA1AS or HAdA1MM for 24 hours, with a fresh change of media and oligonucleotides after 12 hours of incubation. Following 24 hour exposure to the oligonucleotides, cells were harvested and their RNA extracted by standard procedures. A 21-mer probe corresponding to the region of mRNA targeted by the anti-sense (and therefore having the same sequence as the anti-sense, but not phosphorothioated) was synthesized and used to probe northern blots of RNA prepared from HAdA1AS-treated, HAdA1MM-treated and non-treated HTB-54 cells. These blots showed clearly that HAdA1AS but not HAdA1MM effectively reduced human adenosine receptor mRNA by >50%. This result showed that HAdA1AS is a good candidate for an anti-asthma drug since it depletes intracellular mRNA for the adenosine A₁ receptor, which is involved in asthma.

### Example 3

### In Vivo Efficacy of A₁ Adenosine Receptor Anti-sense Oligonucleotides

A fortuitous homology between the rabbit and human DNA sequences within the adenosine A₁ gene overlapping the initiation codon permitted the use of the phosphorothioate anti-sense oligonucleotides initially designed for use against the human adenosine A₁ receptor in a rabbit model.

Neonatal New Zealand white Pasteurella-free rabbits were immunized intraperitoneally within 24 hours of birth with 312 antigen units/mL house dust mite (D. farinae) extract (Berkeley Biologicals, Berkeley, Calif.), mixed with 10% kaolin. Immunizations were repeated weekly for the first month and then biweekly for the next 2 months. At 3-4 months of age, eight sensitized rabbits were anesthetized and relaxed with a mixture of ketamine hydrochloride (44 mg/kg) and acepromazine maleate (0.4 mg/kg) administered intramuscularly.

The rabbits were then laid supine in a comfortable position on a small molded, padded animal board and intubated with a 4.0-mm intratracheal tube (Mallinkrodt, Inc., Glens Falls, N.Y.). A polyethylene catheter of external diameter 2.4 mm with an attached latex balloon was passed into the esophagus and maintained at the same distance (approximately 16 cm) from the mouth throughout the experiments. The intratracheal tube was attached to a heated Fleisch pneumotachograph (size 00; DOM Medical, Richmond, Va.), and flow was measured using a Validyne differential pressure transducer (Model DP-45161927; Validyne Engineering Corp., Northridge, Calif.) driven by a Gould carrier amplifier (Model 11-4113; Gould Electronic, Cleveland, Ohio). The esophageal balloon was attached to one side of the differential pressure transducer, and the outflow of the intratracheal tube was connected to the opposite side of the pressure transducer to allow recording of transpulmonary pressure. Flow was integrated to give a continuous tidal volume, and measurements of total lung resistance (RL) and dynamic compliance (Cdyn) were calculated at isovolumetric and flow zero points, respectively, using an automated respiratory analyzer (Model 6; Buxco, Sharon, Conn.).

Animals were randomized and on Day 1 pretreatment values for PC50 were obtained for aerosolized adenosine. Anti-sense (HAdA1AS) or mismatched control (HAdA1MM) oligonucleotides were dissolved in sterile physiological saline at a concentration of 5000 μg (5 mg) per 1.0 ml. Animals were subsequently administered the aerosolized anti-sense or mismatch oligonucleotide via the intratracheal tube (approximately 5000 μg in a volume of 1.0 ml), twice daily for two days. Aerosols of either saline, adenosine, or anti-sense or mismatch oligonucleotides were generated by an ultrasonic nebulizer (DeVilbiss, Somerset, Pa.), producing aerosol droplets 80% of which were smaller than 5 μm in diameter.

In the first arm of the experiment, four randomly selected allergic rabbits were administered anti-sense oligonucleotide and four the mismatched control oligonucleotide. On the morning of the third day, PC50 values (the concentration of aerosolized adenosine in mg/ml required to reduce the dynamic compliance of the bronchial airway 50% from the baseline value) were obtained and compared to PC50 values obtained for these animals prior to exposure to oligonucleotide.

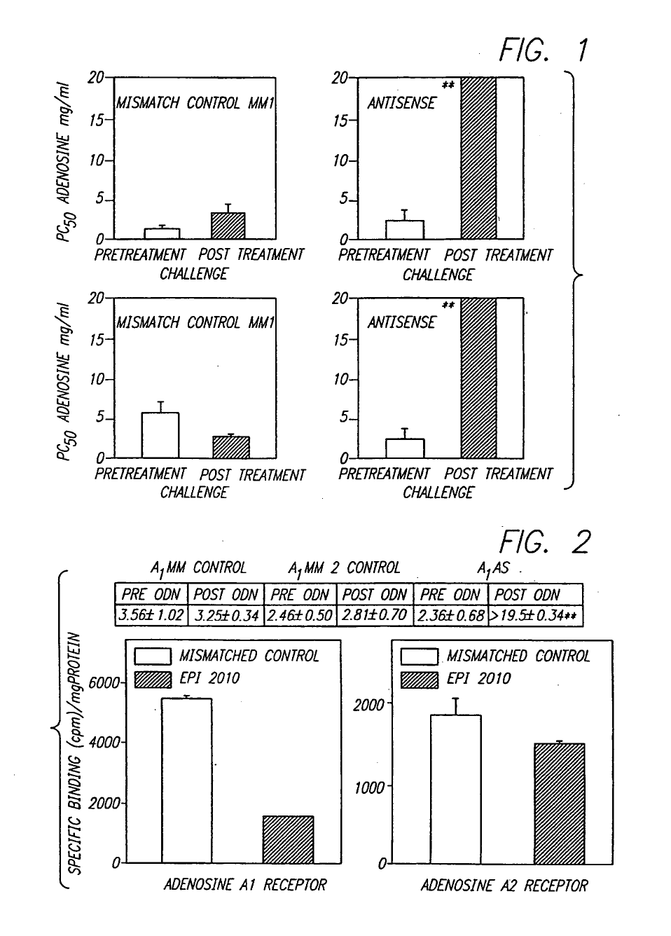

Following a 1 week interval, animals were crossed over, with those previously administered mismatch control oligonucleotide now administered anti-sense oligonucleotide, and those previously treated with anti-sense oligonucleotide now administered mismatch control oligonucleotide. Treatment methods and measurements were identical to those employed in the first arm of the experiment. It should be noted that in six of the eight animals treated with anti-sense oligonucleotide, adenosine-mediated bronchoconstriction could not be obtained up to the limit of solubility of adenosine, 20 mg/ml. For the purpose of calculation, PC50 values for these animals were set at 20 mg/ml. The values given therefore represent a minimum figure for anti-sense effectiveness. Actual effectiveness was higher. The results of this experiment are illustrated in both FIG. 1 and Table 1.

TABLE 1
Adenosine A₁ Receptor Anti-sense Oligonucleotide
Effect upon PC50 Values in Asthmatic Rabbits

| | | A₁ Receptor | |
|---|---|---|---|
| Mismatch Control | | Anti-sense Oligonucleotide | |
| Pre | Post | Pre | Post |
| oligonucleotide | oligonucleotide | oligonucleotide | oligonucleotide |
| 3.56 ± 1.02 | 5.16 ± 1.93 | 2.36 ± 0.68 | >19.5 ± 0.34** |

Results are presented as the mean (n = 8) ± SEM. Significance was determined by repeated-measures analysis of variance (ANOVA), and Tukey's protected t test. **Significantly different from all other groups, p < 0.01.

In both arms of the experiment, animals receiving the anti-sense oligonucleotide showed an order of magnitude increase in the dose of aerosolized adenosine required to reduce dynamic compliance of the lung by 50%. No effect of the mismatched control oligonucleotide upon PC50 values was observed. No toxicity was observed in any animal receiving either anti-sense or control inhaled oligonucleotide.

These results show clearly that the lung has exceptional potential as a target for anti-sense oligonucleotide-based therapeutic intervention in lung disease. They further show, in a model system which closely resembles human asthma, that down regulation of the adenosine A₁ receptor largely eliminates adenosine-mediated bronchoconstriction in asthmatic airways. Bronchial hyper-responsiveness in the allergic rabbit model of human asthma is an excellent endpoint for anti-sense intervention since the tissues involved in this response lie near to the point of contact with aerosolized oligonucleotides, and the model closely simulates an important human disease.

### Example 4

### Specificity of A₁-adenosine Receptor Anti-sense Oligonucleotide

At the conclusion of the crossover experiment of Example 3, airway smooth muscle from all rabbits was quantitatively analyzed for adenosine A₁ receptor number. As a control for the specificity of the anti-sense oligonucleotide, adenosine A₂ receptors, which should not have been affected, were also quantified.

Airway smooth muscle tissue was dissected from each rabbit and a membrane fraction prepared according to described methods (Kleinstein, J., and Glossmann, H., Naunyn-Schmiedeberg's Arch. Pharmacol. 305, 191-200 (1978), with slight modifications. Crude plasma membrane preparations were stored at −70° C. until the time of assay. Protein content was determined by the method of Bradford (M. Bradford, Anal. Biochem. 72, 240-254 (1976)). Frozen plasma membranes were thawed at room temperature and were incubated with 0.2 U/ml adenosine deaminase for 30 minutes at 37° C. to remove endogenous adenosine. The binding of [³H] DPCPX (A₁ receptor-specific) or [³H] CGS-21680 (A₂ receptor-specific) was measured as previously described. See, Ali, S., et al., J. Pharmacol. Exp. Ther. 268, 1328-1334 (1994); S. Ali et al., Am. J. Physiol. 266, L271-277 (1994).

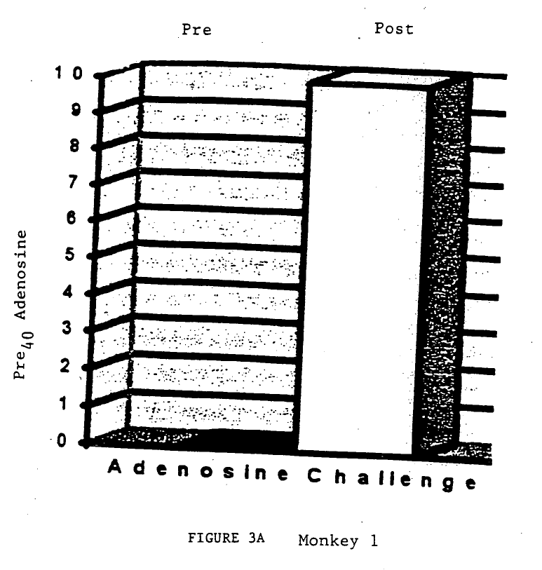

As illustrated in both FIG. 2 and Table 2, animals treated with adenosine A₁ anti-sense oligonucleotide in the crossover experiment had a nearly 75% decrease in A₁ receptor number compared to controls, as assayed by specific binding of the A₁-specific antagonist DPCPX. There was no change in adenosine A₂ receptor number, as assayed by specific binding of the A₂ receptor-specific agonist 2-[p-(2-carboxyethyl)-phenethylamino]-5′-(N-ethylcarboxamido)adenosine (CGS-21680).

TABLE 2
Specificity or Action of Adenosine A₁
Receptor Anti-sense Oligonucleotide

| | Mismatch Control | A₁-Anti-sense |
|---|---|---|
| | Oligonucleotide | Oligonucleotide |
| | (Mean ± SD) | (Mean ± SD) |
| | n = 8 | n = 8 |
| A₁-Specific Binding | 1,105 ± 48** | 293 ± 18 |
| A₂-Specific Binding | 302 ± 22** | 442 ± 171 |

Significance was determined by repeated-measures analysis of variance (ANOVA), and Tukey's protected t test. **Significantly different from mismatch control, p < 0.01.

### Example 5

### In Vivo Response to Adenosine Challenge with & without Oligo I Pretreatment

Two hyper responsive monkeys (ascaris sensitive) were challenged with inhaled adenosine, with and without pre-treatment with anti-sense oligo I (SEQ.ID NO:1). The PC₄₀ adenosine was calculated from the data collected as being equivalent to that amount of adenosine in mg that causes a 40% decrease in dynamic compliance in hyper-responsive airways.

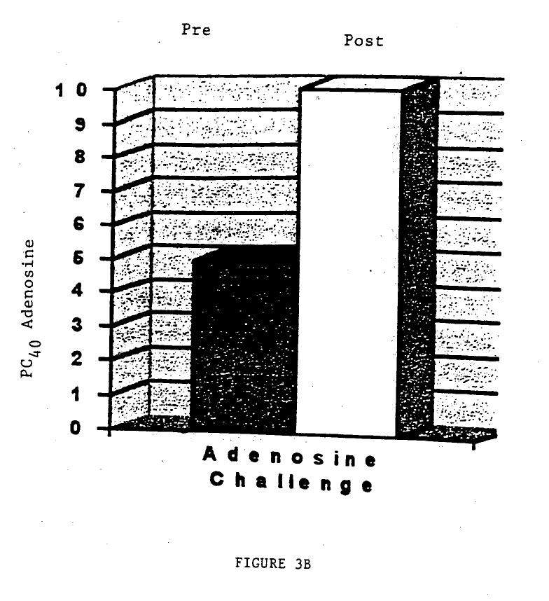

The Oligo I (SEQ. ID NO:1; EPI 2010) was subsequently administered at 10 mg/day for 2 days by inhalation. On the third day, PC adenosine was again measured. The results are shown in FIG. 3 accompanying this patent. The left bar shows the PC₄₀ adenosine value prior to treatment with Oligo I whereas the right bar shows the PC₄₀ adenosine taken after administration of Oligo I. As can be seen in FIG. 3, any sensitivity to adenosine was completely eliminated by the administration of the oligo of this invention in one animal, and substantially reduced in the second.

The foregoing examples are illustrative of the present invention, BUT are not to be construed as limiting thereof. The invention is further defined by the following claims, with equivalents of the claims to be included therein.

1-107. (Cancelled).

108. A pharmaceutical composition comprising an antisense oligonucleotide effective to alleviate hyper-responsiveness to adenosine or increased levels of adenosine, or to alleviate bronchoconstriction, asthma, or lung allergy, wherein said antisense oligonucleotide is 4 to 60 nucleotides long and comprises 15% or less adenosine, wherein said antisense oligonucleotide is antisense to a target polynucleotide, wherein said pharmaceutical composition is of a respirable or inhalable particle size.

109. The pharmaceutical composition of claim 108, wherein the antisense oligonucleotide comprises 10% or less adenosine.

110. The pharmaceutical composition of claim 109, wherein the antisense oligonucleotide comprises 5% or less adenosine.

111. The pharmaceutical composition of claim 110, wherein the antisense oligonucleotide is adenosine-free.

112. The pharmaceutical composition of claim 108, wherein the antisense oligonucleotide is 9 to 51 nucleotides long.

113. The pharmaceutical composition of claim 112, wherein the antisense oligonucleotide is 18 or 21 nucleotides long.

114. The pharmaceutical composition of claim 108, wherein the antisense oligonucleotide is antisense to the initiation codon, the coding region or the 5′ or 3′ intron-exon junction of a gene encoding a protein associated with hyper-responsiveness to adenosine, hyper-responsiveness to increased levels of adenosine, hyper-responsiveness to increased levels of an adenosine receptor, bronchoconstriction, asthma, lung allergy, or lung inflammation, or is antisense to the corresponding mRNA thereof.

115. The pharmaceutical composition of claim 108, wherein the particle size is about 0.5 μm to about 10 μm in size.

116. The pharmaceutical composition of claim 108, wherein the particle size is 10 μm to 500 μm in size.

117. The pharmaceutical composition of claim 108, further comprising a surfactant.

118. The pharmaceutical composition of claim 108, wherein the hyper-responsiveness to adenosine, hyper-responsiveness to increased levels of adenosine, hyper-responsiveness to increased levels of an adenosine receptor, bronchoconstriction, asthma, lung allergy, or lung inflammation is associated with allergy, chronic obstructive pulmonary disease, asthma, acute respiratory distress syndrome, respiratory distress syndrome, or a side effect of adenosine administration.

119. The pharmaceutical composition of claim 108, wherein the antisense oligonucleotide is administered in an amount of about 0.01 to about 150 mg/kg body weight.

120. The pharmaceutical composition of claim 108, wherein the antisense oligonucleotide is antisense to the initiation codon, the coding region or the 5′ or 3′ intron-exon junctions of a gene encoding an adenosine A₁ receptor, adenosine A_{2b} receptor or adenosine A₃ receptor.

121. The pharmaceutical composition of claim 108, wherein said antisense oligonucleotide comprises a sequence of SEQ ID NO: 1, SEQ ID NO: 3, SEQ ID NO: 5 or SEQ ID NO: 7 to SEQ ID NO: 996.

122. The pharmaceutical composition of claim 108, wherein said antisense oligonucleotide comprises wherein at least one mononucleotide linked or modified by one or more of phosphorothioate, phosphorodithioate, methylphosphonate, phosphoramidate, boranophosphate, phosphotriester, formacetal, 2′-O-methyl, thioformacetal, 5′-thioether, carbonate, 5′-N-carbamate,sulfate, sulfonate, sulfamate, sulfonamide, sulfone, sulfite, sulfoxide, sulfide, hydroxylamine, methylene (methylimino) and methyleneoxy(methylimino), terminal 1,3-propanediol, terminal dodecanol, 2′-O-methoxyethyl, C-5-propynyl pyrimidine, C-5 methyl cytidine, C-5 ethynyl pyrimidine, 2′ propoxy, C-18 amine, N3′-P5 phosphoramidates, 3′-alkylamino, 2′-fluoro pyrimidine, 5-fluoro pyrimidine, 5-iodo pyrimidine, 5-bromo pyrimidine, 2′-borano, C-5 hexynyl pyrimidine, 2′-O-(2-methoxy)ethyl, 2′-O-aminopropyl, 5-(phenylethyl) or a peptide nucleic acid interbase linkages or conjugated to a polyethylene glycol, cholesterol, cholesteryl, dehydroepiandrosterone, dehydroepiandrosterone sulfate, dehydroepiandrosterone sulfatide, ubiquinone, dolichol, poly L-lysine, sulfatidic acid or a fatty acid.


SEQUENCE LISTING (996 sequences)

SEQ ID NO: 1 - DNA, 21 bp, Homo sapiens
gatggagggcggcatggcggg

SEQ ID NO: 2 - DNA, 21 bp, Homo sapiens
gtagcaggcggggatgggggc

SEQ ID NO: 3 - DNA, 18 bp, Homo sapiens
gttgttgggcatcttgcc

SEQ ID NO: 4 - DNA, 18 bp, Homo sapiens
gtacttgcggatctaggc

SEQ ID NO: 5 - DNA, 18 bp, Homo sapiens
gtgggcctagctctcgcc

SEQ ID NO: 6 - DNA, 18 bp, Homo sapiens
gtcggggtacctgtcggc

SEQ ID NO: 7 - DNA, 51 bp, Homo sapiens
ggcggcctggaaagctgagatggagggcggcatggcgggcacaggctgggc

SEQ ID NO: 8 - DNA, 50 bp, Homo sapiens
ggcggcctggaaagctgagatggagggcggcatggcgggcacaggctggg

SEQ ID NO: 9 - DNA, 49 bp, Homo sapiens
ggcggcctggaaagctgagatggagggcggcatggcgggcacagggtgg

SEQ ID NO: 10 - DNA, 48 bp, Homo sapiens
ggcggcctggaaagctgagatggagggcggcatggcgggcacaggctg

SEQ ID NO: 11 - DNA, 47 bp, Homo sapiens
ggcggcctggaaagctgagatggagggcggcatggcgggcacagggt

SEQ ID NO: 12 - DNA, 46 bp, Homo sapiens
ggcggcctggaaagctgagatggagggcggcatggcgggcacaggg

SEQ ID NO: 13 - DNA, 45 bp, Homo sapiens
ggcggcctggaaagctgagatggagggcggcatggcgggcacagg

SEQ ID NO: 14 - DNA, 44 bp, Homo sapiens
ggcggcctggaaagctgagatggagggcggcatggcgggcacag

SEQ ID NO: 15 - DNA, 43 bp, Homo sapiens
ggcggcctggaaagctgagatggagggcggcatggcgggcaca

SEQ ID NO: 16 - DNA, 42 bp, Homo sapiens
ggcggcctggaaagctgagatggagggcggcatggcgggcac

SEQ ID NO: 17 - DNA, 41 bp, Homo sapiens
ggcggcctggaaagctgagatggagggcggcatggcgggca

SEQ ID NO: 18 - DNA, 40 bp, Homo sapiens
ggcggcctggaaagctgagatggagggcggcatggcgggg

SEQ ID NO: 19 - DNA, 39 bp, Homo sapiens
ggcggcctggaaagctgagatggagggcggcatggcggg

SEQ ID NO: 20 - DNA, 38 bp, Homo sapiens
ggcggcctggaaagctgagatggagggcggcatggcgg

SEQ ID NO: 21 - DNA, 37 bp, Homo sapiens
ggcggcctggaaagctgagatggagggcggcatggcg

SEQ ID NO: 22 - DNA, 36 bp, Homo sapiens
ggcggcctggaaagctgagatggagggcggcatggc

SEQ ID NO: 23 - DNA, 35 bp, Homo sapiens
ggcggcctggaaagctgagatggagggcggcatgg

SEQ ID NO: 24 - DNA, 34 bp, Homo sapiens
ggcggcctggaaagctgagatggagggcggcatg

SEQ ID NO: 25 - DNA, 33 bp, Homo sapiens
ggcggcctggaaagctgagatggagggcggcat

SEQ ID NO: 26 - DNA, 32 bp, Homo sapiens
ggcggcctggaaagctgagatggagggcggca

SEQ ID NO: 27 - DNA, 30 bp, Homo sapiens
ggcggcctggaaagctgagatggagggcgg

SEQ ID NO: 28 - DNA, 30 bp, Homo sapiens; identical to SEQ ID NO: 27

SEQ ID NO: 29 - DNA, 29 bp, Homo sapiens
ggcggcctggaaagctgagatggagggcg

SEQ ID NO: 30 - DNA, 28 bp, Homo sapiens
ggcggcctggaaagctgagatggagggc

SEQ ID NO: 31 - DNA, 27 bp, Homo sapiens
ggcggcctggaaagctgagatggaggg

SEQ ID NO: 32 - DNA, 26 bp, Homo sapiens
ggcggcctggaaagctgagatggagg

SEQ ID NO: 33 - DNA, 25 bp, Homo sapiens
ggcggcctggaaagctgagatggag

SEQ ID NO: 34 - DNA, 24 bp, Homo sapiens
ggcggcctggaaagctgagatgga

SEQ ID NO: 35 - DNA, 23 bp, Homo sapiens
ggcggcctggaaagctgagatgg

SEQ ID NO: 36 - DNA, 22 bp, Homo sapiens
ggcggcctggaaagctgagatg

SEQ ID NO: 37 - DNA, 21 bp, Homo sapiens
ggcggcctggaaagctgagat

SEQ ID NO: 38 - DNA, 20 bp, Homo sapiens
ggcggcctggaaagctgaga

SEQ ID NO: 39 - DNA, 19 bp, Homo sapiens
ggcggcctggaaagctgag

SEQ ID NO: 40 - DNA, 18 bp, Homo sapiens
ggcggcctggaaagctga

SEQ ID NO: 41 - DNA, 17 bp, Homo sapiens
ggcggcctggaaagctg

SEQ ID NO: 42 - DNA, 16 bp, Homo sapiens
ggcggcctggaaagct

SEQ ID NO: 43 - DNA, 15 bp, Homo sapiens
ggcggcctggaaagc

SEQ ID NO: 44 - DNA, 14 bp, Homo sapiens
ggcggcctggaaag

SEQ ID NO: 45 - DNA, 13 bp, Homo sapiens
ggcggcctggaaa

SEQ ID NO: 46 - DNA, 12 bp, Homo sapiens
ggcggcctggaa

SEQ ID NO: 47 - DNA, 11 bp, Homo sapiens
ggcggcctgga

SEQ ID NO: 48 - DNA, 10 bp, Homo sapiens
ggcggcctgg

SEQ ID NO: 49 - DNA, 50 bp, Homo sapiens
gcggcctggaaagctgagatggagggcggcatggcgggcacaggctgggc

SEQ ID NO: 50 - DNA, 49 bp, Homo sapiens
gcggcctggaaagctgagatggagggcggcatggcgggcacaggctggg

SEQ ID NO: 51 - DNA, 48 bp, Homo sapiens
gcggcctggaaagctgagatggagggcggcatggcgggcacaggctgg

SEQ ID NO: 52 - DNA, 47 bp, Homo sapiens
gcggcctggaaagctgagatggagggcggcatggcgggcacaggctg

SEQ ID NO: 53 - DNA, 46 bp, Homo sapiens
gcggcctggaaagctgagatggagggcggcatggcgggcacaggct

SEQ ID NO: 54 - DNA, 45 bp, Homo sapiens
gcggcctggaaagctgagatggagggcggcatggcgggcacaggc

SEQ ID NO: 55 - DNA, 44 bp, Homo sapiens
gcggcctggaaagctgagatggagggcggcatggcgggcacagg

SEQ ID NO: 56 - DNA, 43 bp, Homo sapiens
gcggcctggaaagctgagatggagggcggcatggcgggcacag

SEQ ID NO: 57 - DNA, 42 bp, Homo sapiens
gcggcctggaaagctgagatggagggcggcatggcgggcaca

SEQ ID NO: 58 - DNA, 41 bp, Homo sapiens
gcggcctggaaagctgagatggagggcggcatggcgggcac

SEQ ID NO: 59 - DNA, 40 bp, Homo sapiens
gcggcctggaaagctgagatggagggcggcatggcgggca

SEQ ID NO: 60 - DNA, 39 bp, Homo sapiens
gcggcctggaaagctgagatggagggcggcatggcgggc

SEQ ID NO: 61 - DNA, 38 bp, Homo sapiens
gcggcctggaaagctgagatggagggcggcatggcggg

SEQ ID NO: 62 - DNA, 37 bp, Homo sapiens
gcggcctggaaagctgagatggagggcggcatggcgg

SEQ ID NO: 63 - DNA, 36 bp, Homo sapiens
gcggcctggaaagctgagatggagggcggcatggcg

SEQ ID NO: 64 - DNA, 35 bp, Homo sapiens
gcggcctggaaagctgagatggagggcggcatggc

SEQ ID NO: 65 - DNA, 34 bp, Homo sapiens
gcggcctggaaagctgagatggagggcggcatgg

SEQ ID NO: 66 - DNA, 33 bp, Homo sapiens
gcggcctggaaagctgagatggagggcggcatg

SEQ ID NO: 67 - DNA, 32 bp, Homo sapiens
gcggcctggaaagctgagatggagggcggcat

SEQ ID NO: 68 - DNA, 31 bp, Homo sapiens
gcggcctggaaagctgagatggagggcggca

SEQ ID NO: 69 - DNA, 30 bp, Homo sapiens
gcggcctggaaagctgagatggagggcggc

SEQ ID NO: 70 - DNA, 29 bp, Homo sapiens
gcggcctggaaagctgagatggagggcgg

SEQ ID NO: 71 - DNA, 28 bp, Homo sapiens
gcggcctggaaagctgagatggagggcg

SEQ ID NO: 72 - DNA, 27 bp, Homo sapiens
gcggcctggaaagctgagatggagggc

SEQ ID NO: 73 - DNA, 26 bp, Homo sapiens
gcggcctggaaagctgagatggaggg

SEQ ID NO: 74 - DNA, 25 bp, Homo sapiens
gcggcctggaaagctgagatggagg

SEQ ID NO: 75 - DNA, 24 bp, Homo sapiens
gcggcctggaaagctgagatggag

SEQ ID NO: 76 - DNA, 23 bp, Homo sapiens
gcggcctggaaagctgagatgga

SEQ ID NO: 77 - DNA, 22 bp, Homo sapiens
gcggcctggaaagctgagatgg

SEQ ID NO: 78 - DNA, 21 bp, Homo sapiens
gcggcctggaaagctgagatg

SEQ ID NO: 79 - DNA, 20 bp, Homo sapiens
gcggcctggaaagctgagat

SEQ ID NO: 80 - DNA, 19 bp, Homo sapiens
gcggcctggaaagctgaga

SEQ ID NO: 81 - DNA, 18 bp, Homo sapiens
gcggcctggaaagctgag

SEQ ID NO: 82 - DNA, 17 bp, Homo sapiens
gcggcctggaaagctga

SEQ ID NO: 83 - DNA, 16 bp, Homo sapiens
gcggcctggaaagctg

SEQ ID NO: 84 - DNA, 15 bp, Homo sapiens
gcggcctggaaagct

SEQ ID NO: 85 - DNA, 14 bp, Homo sapiens
gcggcctggaaagc

SEQ ID NO: 86 - DNA, 13 bp, Homo sapiens
gcggcctggaaag

SEQ ID NO: 87 - DNA, 12 bp, Homo sapiens
gcggcctggaaa

SEQ ID NO: 88 - DNA, 11 bp, Homo sapiens
gcggcctggaa

SEQ ID NO: 89 - DNA, 10 bp, Homo sapiens
gcggcctgga

SEQ ID NO: 90 - DNA, 49 bp, Homo sapiens
cggcctggaaagctgagatggagggcggcatggcgggcacaggctgggc

SEQ ID NO: 91 - DNA, 48 bp, Homo sapiens
cggcctggaaagctgagatggagggcggcatggcgggcacaggctggg

SEQ ID NO: 92 - DNA, 47 bp, Homo sapiens
cggcctggaaagctgagatggagggcggcatggcgggcacaggctgg

SEQ ID NO: 93 - DNA, 46 bp, Homo sapiens
cggcctggaaagctgagatggagggcggcatggcgggcacaggctg

SEQ ID NO: 94 - DNA, 45 bp, Homo sapiens
cggcctggaaagctgagatggagggcggcatggcgggcacaggct

SEQ ID NO: 95 - DNA, 44 bp, Homo sapiens
cggcctggaaagctgagatggagggcggcatggcgggcacaggc

SEQ ID NO: 96 - DNA, 43 bp, Homo sapiens
cggcctggaaagctgagatggagggcggcatggcgggcacagg

SEQ ID NO: 97 - DNA, 42 bp, Homo sapiens
cggcctggaaagctgagatggagggcggcatggcgggcacag

SEQ ID NO: 98 - DNA, 41 bp, Homo sapiens
cggcctggaaagctgagatggagggcggcatggcgggcaca

SEQ ID NO: 99 - DNA, 40 bp, Homo sapiens
cggcctggaaagctgagatggagggcggcatggcgggcac

SEQ ID NO: 100 - DNA, 39 bp, Homo sapiens
cggcctggaaagctgagatggagggcggcatggcgggca

SEQ ID NO: 101 - DNA, 38 bp, Homo sapiens
cggcctggaaagctgagatggagggcggcatggcgggc

SEQ ID NO: 102 - DNA, 37 bp, Homo sapiens
cggcctggaaagctgagatggagggcggcatggcggg

SEQ ID NO: 103 - DNA, 36 bp, Homo sapiens
cggcctggaaagctgagatggagggcggcatggcgg

SEQ ID NO: 104 - DNA, 35 bp, Homo sapiens
cggcctggaaagctgagatggagggcggcatggcg

SEQ ID NO: 105 - DNA, 34 bp, Homo sapiens
cggcctggaaagctgagatggagggcggcatggc

SEQ ID NO: 106 - DNA, 33 bp, Homo sapiens
cggcctggaaagctgagatggagggcggcatgg

SEQ ID NO: 107 - DNA, 32 bp, Homo sapiens
cggcctggaaagctgagatggagggcggcatg

SEQ ID NO: 108 - DNA, 31 bp, Homo sapiens
cggcctggaaagctgagatggagggcggcat

SEQ ID NO: 109 - DNA, 30 bp, Homo sapiens
cggcctggaaagctgagatggagggcggca

SEQ ID NO: 110 - DNA, 29 bp, Homo sapiens
cggcctggaaagctgagatggagggcggc

SEQ ID NO: 111 - DNA, 28 bp, Homo sapiens
cggcctggaaagctgagatggagggcgg

SEQ ID NO: 112 - DNA, 27 bp, Homo sapiens
cggcctggaaagctgagatggagggcg

SEQ ID NO: 113 - DNA, 26 bp, Homo sapiens
cggcctggaaagctgagatggagggc

SEQ ID NO: 114 - DNA, 25 bp, Homo sapiens
cggcctggaaagctgagatggaggg

SEQ ID NO: 115 - DNA, 24 bp, Homo sapiens
cggcctggaaagctgagatggagg

SEQ ID NO: 116 - DNA, 23 bp, Homo sapiens
cggcctggaaagctgagatggag

SEQ ID NO: 117 - DNA, 22 bp, Homo sapiens
cggcctggaaagctgagatgga

SEQ ID NO: 118 - DNA, 21 bp, Homo sapiens
cggcctggaaagctgagatgg

SEQ ID NO: 119 - DNA, 20 bp, Homo sapiens
cggcctggaaagctgagatg

SEQ ID NO: 120 - DNA, 19 bp, Homo sapiens
cggcctggaaagctgagat

SEQ ID NO: 121 - DNA, 18 bp, Homo sapiens
cggcctggaaagctgaga

SEQ ID NO: 122 - DNA, 17 bp, Homo sapiens
cggcctggaaagctgag

SEQ ID NO: 123 - DNA, 16 bp, Homo sapiens
cggcctggaaagctga

SEQ ID NO: 124 - DNA, 15 bp, Homo sapiens
cggcctggaaagctg

SEQ ID NO: 125 - DNA, 14 bp, Homo sapiens
cggcctggaaagct

SEQ ID NO: 126 - DNA, 13 bp, Homo sapiens
cggcctggaaagc

SEQ ID NO: 127 - DNA, 12 bp, Homo sapiens
cggcctggaaag

SEQ ID NO: 128 - DNA, 11 bp, Homo sapiens
cggcctggaaa

SEQ ID NO: 129 - DNA, 10 bp, Homo sapiens
cggcctggaa

SEQ ID NO: 130 - DNA, 48 bp, Homo sapiens
ggcctggaaagctgagatggagggcggcatggcgggcacaggctgggc

SEQ ID NO: 131 - DNA, 47 bp, Homo sapiens
ggcctggaaagctgagatggagggcggcatggcgggcacaggctggg

SEQ ID NO: 132 - DNA, 46 bp, Homo sapiens
ggcctggaaagctgagatggagggcggcatggcgggcacaggctgg

SEQ ID NO: 133 - DNA, 45 bp, Homo sapiens
ggcctggaaagctgagatggagggcggcatggcgggcacaggctg

SEQ ID NO: 134 - DNA, 44 bp, Homo sapiens
ggcctggaaagctgagatggagggcggcatggcgggcacaggct

SEQ ID NO: 135 - DNA, 43 bp, Homo sapiens
ggcctggaaagctgagatggagggcggcatggcgggcacaggc

SEQ ID NO: 136 - DNA, 42 bp, Homo sapiens
ggcctggaaagctgagatggagggcggcatggcgggcacagg

SEQ ID NO: 137 - DNA, 41 bp, Homo sapiens
ggcctggaaagctgagatggagggcggcatggcgggcacag

SEQ ID NO: 138 - DNA, 40 bp, Homo sapiens
ggcctggaaagctgagatggagggcggcatggcgggcaca

SEQ ID NO: 139 - DNA, 39 bp, Homo sapiens
ggcctggaaagctgagatggagggcggcatggcgggcac

SEQ ID NO: 140 - DNA, 38 bp, Homo sapiens
ggcctggaaagctgagatggagggcggcatggcgggca

SEQ ID NO: 141 - DNA, 37 bp, Homo sapiens
ggcctggaaagctgagatggagggcggcatggcgggc

SEQ ID NO: 142 - DNA, 36 bp, Homo sapiens
ggcctggaaagctgagatggagggcggcatggcggg

SEQ ID NO: 143 - DNA, 35 bp, Homo sapiens
ggcctggaaagctgagatggagggcggcatggcgg

SEQ ID NO: 144 - DNA, 34 bp, Homo sapiens
ggcctggaaagctgagatggagggcggcatggcg

SEQ ID NO: 145 - DNA, 33 bp, Homo sapiens
ggcctggaaagctgagatggagggcggcatggc

SEQ ID NO: 146 - DNA, 32 bp, Homo sapiens
ggcctggaaagctgagatggagggcggcatgg

SEQ ID NO: 147 - DNA, 31 bp, Homo sapiens
ggcctggaaagctgagatggagggcggcatg

SEQ ID NO: 148 - DNA, 30 bp, Homo sapiens
ggcctggaaagctgagatggagggcggcat

SEQ ID NO: 149 - DNA, 29 bp, Homo sapiens
ggcctggaaagctgagatggagggcggca

SEQ ID NO: 150 - DNA, 28 bp, Homo sapiens
ggcctggaaagctgagatggagggcggc

SEQ ID NO: 151 - DNA, 27 bp, Homo sapiens
ggcctggaaagctgagatggagggcgg

SEQ ID NO: 152 - DNA, 26 bp, Homo sapiens
ggcctggaaagctgagatggagggcg

SEQ ID NO: 153 - DNA, 25 bp, Homo sapiens
ggcctggaaagctgagatggagggc

SEQ ID NO: 154 - DNA, 24 bp, Homo sapiens
ggcctggaaagctgagatggaggg

SEQ ID NO: 155 - DNA, 23 bp, Homo sapiens
ggcctggaaagctgagatggagg

SEQ ID NO: 156 - DNA, 22 bp, Homo sapiens
ggcctggaaagctgagatggag

SEQ ID NO: 157 - DNA, 21 bp, Homo sapiens
ggcctggaaagctgagatgga

SEQ ID NO: 158 - DNA, 20 bp, Homo sapiens
ggcctggaaagctgagatgg

SEQ ID NO: 159 - DNA, 19 bp, Homo sapiens
ggcctggaaagctgagatg

SEQ ID NO: 160 - DNA, 18 bp, Homo sapiens
ggcctggaaagctgagat

SEQ ID NO: 161 - DNA, 17 bp, Homo sapiens
ggcctggaaagctgaga

SEQ ID NO: 162 - DNA, 16 bp, Homo sapiens
ggcctggaaagctgag

SEQ ID NO: 163 - DNA, 15 bp, Homo sapiens
ggcctggaaagctga

SEQ ID NO: 164 - DNA, 14 bp, Homo sapiens
ggcctggaaagctg

SEQ ID NO: 165 - DNA, 13 bp, Homo sapiens
ggcctggaaagct

SEQ ID NO: 166 - DNA, 12 bp, Homo sapiens
ggcctggaaagc

SEQ ID NO: 167 - DNA, 11 bp, Homo sapiens
ggcctggaaag

SEQ ID NO: 168 - DNA, 10 bp, Homo sapiens
ggcctggaaa

SEQ ID NO: 169 - DNA, 47 bp, Homo sapiens
gcctggaaagctgagatggagggcggcatggcgggcacaggctgggc

SEQ ID NO: 170 - DNA, 46 bp, Homo sapiens
gcctggaaagctgagatggagggcggcatggcgggcacaggctggg

SEQ ID NO: 171 - DNA, 45 bp, Homo sapiens
gcctggaaagctgagatggagggcggcatggcgggcacaggctgg

SEQ ID NO: 172 - DNA, 44 bp, Homo sapiens
gcctggaaagctgagatggagggcggcatggcgggcacaggctg

SEQ ID NO: 173 - DNA, 43 bp, Homo sapiens
gcctggaaagctgagatggagggcggcatggcgggcacaggct

SEQ ID NO: 174 - DNA, 42 bp, Homo sapiens
gcctggaaagctgagatggagggcggcatggcgggcacaggc

SEQ ID NO: 175 - DNA, 41 bp, Homo sapiens
gcctggaaagctgagatggagggcggcatggcgggcacagg

SEQ ID NO: 176 - DNA, 40 bp, Homo sapiens
gcctggaaagctgagatggagggcggcatggcgggcacag

SEQ ID NO: 177 - DNA, 39 bp, Homo sapiens
gcctggaaagctgagatggagggcggcatggcgggcaca

SEQ ID NO: 178 - DNA, 38 bp, Homo sapiens
gcctggaaagctgagatggagggcggcatggcgggcac

SEQ ID NO: 179 - DNA, 37 bp, Homo sapiens
gcctggaaagctgagatggagggcggcatggcgggca

SEQ ID NO: 180 - DNA, 36 bp, Homo sapiens
gcctggaaagctgagatggagggcggcatggcgggc

SEQ ID NO: 181 - DNA, 35 bp, Homo sapiens
gcctggaaagctgagatggagggcggcatggcggg

SEQ ID NO: 182 - DNA, 34 bp, Homo sapiens
gcctggaaagctgagatggagggcggcatggcgg

SEQ ID NO: 183 - DNA, 33 bp, Homo sapiens
gcctggaaagctgagatggagggcggcatggcg

SEQ ID NO: 184 - DNA, 32 bp, Homo sapiens
gcctggaaagctgagatggagggcggcatggc

SEQ ID NO: 185 - DNA, 31 bp, Homo sapiens
gcctggaaagctgagatggagggcggcatgg

SEQ ID NO: 186 - DNA, 30 bp, Homo sapiens
gcctggaaagctgagatggagggcggcatg

SEQ ID NO: 187 - DNA, 29 bp, Homo sapiens
gcctggaaagctgagatggagggcggcat

SEQ ID NO: 188 - DNA, 28 bp, Homo sapiens
gcctggaaagctgagatggagggcggca

SEQ ID NO: 189 - DNA, 27 bp, Homo sapiens
gcctggaaagctgagatggagggcggc

SEQ ID NO: 190 - DNA, 26 bp, Homo sapiens
gcctggaaagctgagatggagggcgg

SEQ ID NO: 191 - DNA, 25 bp, Homo sapiens
gcctggaaagctgagatggagggcg

SEQ ID NO: 192 - DNA, 24 bp, Homo sapiens
gcctggaaagctgagatggagggc

SEQ ID NO: 193 - DNA, 23 bp, Homo sapiens
gcctggaaagctgagatggaggg

SEQ ID NO: 194 - DNA, 22 bp, Homo sapiens
gcctggaaagctgagatggagg

SEQ ID NO: 195 - DNA, 21 bp, Homo sapiens
gcctggaaagctgagatggag

SEQ ID NO: 196 - DNA, 20 bp, Homo sapiens
gcctggaaagctgagatgga

SEQ ID NO: 197 - DNA, 19 bp, Homo sapiens
gcctggaaagctgagatgg

SEQ ID NO: 198 - DNA, 18 bp, Homo sapiens
gcctggaaagctgagatg

SEQ ID NO: 199 - DNA, 17 bp, Homo sapiens
gcctggaaagctgagat

SEQ ID NO: 200 - DNA, 16 bp, Homo sapiens
gcctggaaagctgaga

SEQ ID NO: 201 - DNA, 15 bp, Homo sapiens
gcctggaaagctgag

SEQ ID NO: 202 - DNA, 14 bp, Homo sapiens
gcctggaaagctga

SEQ ID NO: 203 - DNA, 13 bp, Homo sapiens
gcctggaaagctg

SEQ ID NO: 204 - DNA, 12 bp, Homo sapiens
gcctggaaagct

SEQ ID NO: 205 - DNA, 11 bp, Homo sapiens
gcctggaaagc

SEQ ID NO: 206 - DNA, 10 bp, Homo sapiens
gcctggaaag

SEQ ID NO: 207 - DNA, 46 bp, Homo sapiens
cctggaaagctgagatggagggcggcatggcgggcacaggctgggc

SEQ ID NO: 208 - DNA, 45 bp, Homo sapiens
cctggaaagctgagatggagggcggcatggcgggcacaggctggg

SEQ ID NO: 209 - DNA, 44 bp, Homo sapiens
cctggaaagctgagatggagggcggcatggcgggcacaggctgg

SEQ ID NO: 210 - DNA, 43 bp, Homo sapiens
cctggaaagctgagatggagggcggcatggcgggcacaggctg

SEQ ID NO: 211 - DNA, 42 bp, Homo sapiens
cctggaaagctgagatggagggcggcatggcgggcacaggct

SEQ ID NO: 212 - DNA, 41 bp, Homo sapiens
cctggaaagctgagatggagggcggcatggcgggcacaggc

SEQ ID NO: 213 - DNA, 40 bp, Homo sapiens
cctggaaagctgagatggagggcggcatggcgggcacagg

SEQ ID NO: 214 - DNA, 39 bp, Homo sapiens
cctggaaagctgagatggagggcggcatggcgggcacag

SEQ ID NO: 215 - DNA, 38 bp, Homo sapiens
cctggaaagctgagatggagggcggcatggcgggcaca

SEQ ID NO: 216 - DNA, 37 bp, Homo sapiens
cctggaaagctgagatggagggcggcatggcgggcac

SEQ ID NO: 217 - DNA, 36 bp, Homo sapiens
cctggaaagctgagatggagggcggcatggcgggca

SEQ ID NO: 218 - DNA, 35 bp, Homo sapiens
cctggaaagctgagatggagggcggcatggcgggc

SEQ ID NO: 219 - DNA, 34 bp, Homo sapiens
cctggaaagctgagatggagggcggcatggcggg

SEQ ID NO: 220 - DNA, 33 bp, Homo sapiens
cctggaaagctgagatggagggcggcatggcgg

SEQ ID NO: 221 - DNA, 32 bp, Homo sapiens
cctggaaagctgagatggagggcggcatggcg

SEQ ID NO: 222 - DNA, 31 bp, Homo sapiens
cctggaaagctgagatggagggcggcatggc

SEQ ID NO: 223 - DNA, 30 bp, Homo sapiens
cctggaaagctgagatggagggcggcatgg

SEQ ID NO: 224 - DNA, 29 bp, Homo sapiens
cctggaaagctgagatggagggcggcatg

SEQ ID NO: 225 - DNA, 28 bp, Homo sapiens
cctggaaagctgagatggagggcggcat

SEQ ID NO: 226 - DNA, 27 bp, Homo sapiens
cctggaaagctgagatggagggcggca

SEQ ID NO: 227 - DNA, 26 bp, Homo sapiens
cctggaaagctgagatggagggcggc

SEQ ID NO: 228 - DNA, 25 bp, Homo sapiens
cctggaaagctgagatggagggcgg

SEQ ID NO: 229 - DNA, 24 bp, Homo sapiens
cctggaaagctgagatggagggcg

SEQ ID NO: 230 - DNA, 23 bp, Homo sapiens
cctggaaagctgagatggagggc

SEQ ID NO: 231 - DNA, 22 bp, Homo sapiens
cctggaaagctgagatggaggg

SEQ ID NO: 232 - DNA, 21 bp, Homo sapiens
cctggaaagctgagatggagg

SEQ ID NO: 233 - DNA, 20 bp, Homo sapiens
cctggaaagctgagatggag

SEQ ID NO: 234 - DNA, 19 bp, Homo sapiens
cctggaaagctgagatgga

SEQ ID NO: 235 - DNA, 18 bp, Homo sapiens
cctggaaagctgagatgg

SEQ ID NO: 236 - DNA, 17 bp, Homo sapiens
cctggaaagctgagatg

SEQ ID NO: 237 - DNA, 16 bp, Homo sapiens
cctggaaagctgagat

SEQ ID NO: 238 - DNA, 15 bp, Homo sapiens
cctggaaagctgaga

SEQ ID NO: 239 - DNA, 14 bp, Homo sapiens
cctggaaagctgag

SEQ ID NO: 240 - DNA, 13 bp, Homo sapiens
cctggaaagctga

SEQ ID NO: 241 - DNA, 12 bp, Homo sapiens
cctggaaagctg

SEQ ID NO: 242 - DNA, 11 bp, Homo sapiens
cctggaaagct

SEQ ID NO: 243 - DNA, 10 bp, Homo sapiens
cctggaaagc

SEQ ID NO: 244 - DNA, 45 bp, Homo sapiens
ctggaaagctgagatggagggcggcatggcgggcacaggctgggc

SEQ ID NO: 245 - DNA, 44 bp, Homo sapiens
ctggaaagctgagatggagggcggcatggcgggcacaggctggg

SEQ ID NO: 246 - DNA, 43 bp, Homo sapiens
ctggaaagctgagatggagggcggcatggcgggcacaggctgg

SEQ ID NO: 247 - DNA, 42 bp, Homo sapiens
ctggaaagctgagatggagggcggcatggcgggcacaggctg

SEQ ID NO: 248 - DNA, 41 bp, Homo sapiens
ctggaaagctgagatggagggcggcatggcgggcacaggct

SEQ ID NO: 249 - DNA, 40 bp, Homo sapiens
ctggaaagctgagatggagggcggcatggcgggcacaggc

SEQ ID NO: 250 - DNA, 39 bp, Homo sapiens
ctggaaagctgagatggagggcggcatggcgggcacagg

SEQ ID NO: 251 - DNA, 38 bp, Homo sapiens
ctggaaagctgagatggagggcggcatggcgggcacag

SEQ ID NO: 252 - DNA, 37 bp, Homo sapiens
ctggaaagctgagatggagggcggcatggcgggcaca

SEQ ID NO: 253 - DNA, 36 bp, Homo sapiens
ctggaaagctgagatggagggcggcatggcgggcac

SEQ ID NO: 254 - DNA, 35 bp, Homo sapiens
ctggaaagctgagatggagggcggcatggcgggca

SEQ ID NO: 255 - DNA, 34 bp, Homo sapiens
ctggaaagctgagatggagggcggcatggcgggc

SEQ ID NO: 256 - DNA, 33 bp, Homo sapiens
ctggaaagctgagatggagggcggcatggcggg

SEQ ID NO: 257 - DNA, 32 bp, Homo sapiens
ctggaaagctgagatggagggcggcatggcgg

SEQ ID NO: 258 - DNA, 31 bp, Homo sapiens
ctggaaagctgagatggagggcggcatggcg

SEQ ID NO: 259 - DNA, 30 bp, Homo sapiens
ctggaaagctgagatggagggcggcatggc

SEQ ID NO: 260 - DNA, 29 bp, Homo sapiens
ctggaaagctgagatggagggcggcatgg

SEQ ID NO: 261 - DNA, 28 bp, Homo sapiens
ctggaaagctgagatggagggcggcatg

SEQ ID NO: 262 - DNA, 27 bp, Homo sapiens
ctggaaagctgagatggagggcggcat

SEQ ID NO: 263 - DNA, 26 bp, Homo sapiens
ctggaaagctgagatggagggcggga

SEQ ID NO: 264 - DNA, 25 bp, Homo sapiens
ctggaaagctgagatggagggcggc

SEQ ID NO: 265 - DNA, 24 bp, Homo sapiens
ctggaaagctgagatggagggcgg

SEQ ID NO: 266 - DNA, 23 bp, Homo sapiens
ctggaaagctgagatggagggcg

SEQ ID NO: 267 - DNA, 22 bp, Homo sapiens
ctggaaagctgagatggagggc

SEQ ID NO: 268 - DNA, 21 bp, Homo sapiens
ctggaaagctgagatggaggg

SEQ ID NO: 269 - DNA, 20 bp, Homo sapiens
gtggaaagctgagatggagg

SEQ ID NO: 270 - DNA, 19 bp, Homo sapiens
ctggaaagctgagatggag

SEQ ID NO: 271 - DNA, 18 bp, Homo sapiens
ctggaaagctgagatgga

SEQ ID NO: 272 - DNA, 17 bp, Homo sapiens
ctggaaagctgagatgg

SEQ ID NO: 273 - DNA, 16 bp, Homo sapiens
ctggaaagctgagatg

SEQ ID NO: 274 - DNA, 15 bp, Homo sapiens
ctggaaagctgagat

SEQ ID NO: 275 - DNA, 14 bp, Homo sapiens
ctggaaagctgaga

SEQ ID NO: 276 - DNA, 13 bp, Homo sapiens
ctggaaagctgag

SEQ ID NO: 277 - DNA, 12 bp, Homo sapiens
ctggaaagctga

SEQ ID NO: 278 - DNA, 11 bp, Homo sapiens
ctggaaagctg

SEQ ID NO: 279 - DNA, 10 bp, Homo sapiens
ctggaaagct

SEQ ID NO: 280 - DNA, 44 bp, Homo sapiens
tggaaagctgagatggagggcggcatggcgggcacaggctgggc

SEQ ID NO: 281 - DNA, 43 bp, Homo sapiens
tggaaagctgagatggagggcggcatggcgggcacaggctggg

SEQ ID NO: 282 - DNA, 42 bp, Homo sapiens
tggaaagctgagatggagggcggcatggcgggcacaggctgg

SEQ ID NO: 283 - DNA, 41 bp, Homo sapiens
tggaaagctgagatggagggcggcatggcgggcacaggctg

SEQ ID NO: 284 - DNA, 40 bp, Homo sapiens
tggaaagctgagatggagggcggcatggcgggcacaggct

SEQ ID NO: 285 - DNA, 39 bp, Homo sapiens
tggaaagctgagatggagggcggcatggcgggcacaggc

SEQ ID NO: 286 - DNA, 38 bp, Homo sapiens
tggaaagctgagatggagggcggcatggcgggcacagg

SEQ ID NO: 287 - DNA, 37 bp, Homo sapiens
tggaaagctgagatggagggcggcatggcgggcacag

SEQ ID NO: 288 - DNA, 36 bp, Homo sapiens
tggaaagctgagatggagggcggcatggcgggcaca

SEQ ID NO: 289 - DNA, 35 bp, Homo sapiens
tggaaagctgagatggagggcggcatggcgggcac

SEQ ID NO: 290 - DNA, 34 bp, Homo sapiens
tggaaagctgagatggagggcggcatggcgggca

SEQ ID NO: 291 - DNA, 33 bp, Homo sapiens
tggaaagctgagatggagggcggcatggcgggc

SEQ ID NO: 292 - DNA, 32 bp, Homo sapiens
tggaaagctgagatggagggcggcatggcggg

SEQ ID NO: 293 - DNA, 31 bp, Homo sapiens
tggaaagctgagatggagggcggcatggcgg

SEQ ID NO: 294 - DNA, 30 bp, Homo sapiens
tggaaagctgagatggagggcggcatggcg

SEQ ID NO: 295 - DNA, 29 bp, Homo sapiens
tggaaagctgagatggagggcggcatggc

SEQ ID NO: 296 - DNA, 28 bp, Homo sapiens
tggaaagctgagatggagggcggcatgg

SEQ ID NO: 297 - DNA, 27 bp, Homo sapiens
tggaaagctgagatggagggcggcatg

SEQ ID NO: 298 - DNA, 26 bp, Homo sapiens
tggaaagctgagatggagggcggcat

SEQ ID NO: 299 - DNA, 25 bp, Homo sapiens
tggaaagctgagatggagggcggca

SEQ ID NO: 300 - DNA, 24 bp, Homo sapiens
tggaaagctgagatggagggcggc

SEQ ID NO: 301 - DNA, 23 bp, Homo sapiens
tggaaagctgagatggagggcgg

SEQ ID NO: 302 - DNA, 22 bp, Homo sapiens
tggaaagctgagatggagggcg

SEQ ID NO: 303 - DNA, 21 bp, Homo sapiens
tggaaagctgagatggagggc

SEQ ID NO: 304 - DNA, 20 bp, Homo sapiens
tggaaagctgagatggaggg

SEQ ID NO: 305 - DNA, 19 bp, Homo sapiens
tggaaagctgagatggagg

SEQ ID NO: 306 - DNA, 18 bp, Homo sapiens
tggaaagctgagatggag

SEQ ID NO: 307 - DNA, 17 bp, Homo sapiens
tggaaagctgagatgga

SEQ ID NO: 308 - DNA, 16 bp, Homo sapiens
tggaaagctgagatgg

SEQ ID NO: 309 - DNA, 15 bp, Homo sapiens
tggaaagctgagatg

SEQ ID NO: 310 - DNA, 14 bp, Homo sapiens
tggaaagctgagat

SEQ ID NO: 311 - DNA, 13 bp, Homo sapiens
tggaaagctgaga

SEQ ID NO: 312 - DNA, 12 bp, Homo sapiens
tggaaagctgag

SEQ ID NO: 313 - DNA, 11 bp, Homo sapiens
tggaaagctga

SEQ ID NO: 314 - DNA, 10 bp, Homo sapiens
tggaaagctg

SEQ ID NO: 315 - DNA, 43 bp, Homo sapiens
ggaaagctgagatggagggcggcatggcgggcacaggctgggc

SEQ ID NO: 316 - DNA, 42 bp, Homo sapiens
ggaaagctgagatggagggcggcatggcgggcacaggctggg

SEQ ID NO: 317 - DNA, 41 bp, Homo sapiens
ggaaagctgagatggagggcggcatggcgggcacaggctgg

SEQ ID NO: 318 - DNA, 40 bp, Homo sapiens
ggaaagctgagatggagggcggcatggcgggcacaggctg

SEQ ID NO: 319 - DNA, 39 bp, Homo sapiens
ggaaagctgagatggagggcggcatggcgggcacaggct

SEQ ID NO: 320 - DNA, 38 bp, Homo sapiens
ggaaagctgagatggagggcggcatggcgggcacaggc

SEQ ID NO: 321 - DNA, 37 bp, Homo sapiens
ggaaagctgagatggagggcggcatggcgggcacagg

SEQ ID NO: 322 - DNA, 36 bp, Homo sapiens
ggaaagctgagatggagggcggcatggcgggcacag

SEQ ID NO: 323 - DNA, 35 bp, Homo sapiens
ggaaagctgagatggagggcggcatggcgggcaca

SEQ ID NO: 324 - DNA, 34 bp, Homo sapiens
ggaaagctgagatggagggcggcatggcgggcac

SEQ ID NO: 325 - DNA, 33 bp, Homo sapiens
ggaaagctgagatggagggcggcatggcgggca

SEQ ID NO: 326 - DNA, 32 bp, Homo sapiens
ggaaagctgagatggagggcggcatggcgggc

SEQ ID NO: 327 - DNA, 31 bp, Homo sapiens
ggaaagctgagatggagggcggcatggcggg

SEQ ID NO: 328 - DNA, 30 bp, Homo sapiens
ggaaagctgagatggagggcggcatggcgg

SEQ ID NO: 329 - DNA, 29 bp, Homo sapiens
ggaaagctgagatggagggcggcatggcg

SEQ ID NO: 330 - DNA, 28 bp, Homo sapiens
ggaaagctgagatggagggcggcatggc

SEQ ID NO: 331 - DNA, 27 bp, Homo sapiens
ggaaagctgagatggagggcggcatgg

SEQ ID NO: 332 - DNA, 26 bp, Homo sapiens
ggaaagctgagatggagggcggcatg

SEQ ID NO: 333 - DNA, 25 bp, Homo sapiens
ggaaagctgagatggagggcggcat

SEQ ID NO: 334 - DNA, 24 bp, Homo sapiens
ggaaagctgagatggagggcggca

SEQ ID NO: 335 - DNA, 23 bp, Homo sapiens
ggaaagctgagatggagggcggc

SEQ ID NO: 336 - DNA, 22 bp, Homo sapiens
ggaaagctgagatggagggcgg

SEQ ID NO: 337 - DNA, 21 bp, Homo sapiens
ggaaagctgagatggagggcg

SEQ ID NO: 338 - DNA, 20 bp, Homo sapiens
ggaaagctgagatggagggc

SEQ ID NO: 339 - DNA, 19 bp, Homo sapiens
ggaaagctgagatggaggg

SEQ ID NO: 340 - DNA, 18 bp, Homo sapiens
ggaaagctgagatggagg

SEQ ID NO: 341 - DNA, 17 bp, Homo sapiens
ggaaagctgagatggag

SEQ ID NO: 342 - DNA, 16 bp, Homo sapiens
ggaaagctgagatgga

SEQ ID NO: 343 - DNA, 15 bp, Homo sapiens
ggaaagctgagatgg

SEQ ID NO: 344 - DNA, 14 bp, Homo sapiens
ggaaagctgagatg

SEQ ID NO: 345 - DNA, 13 bp, Homo sapiens
ggaaagctgagat

SEQ ID NO: 346 - DNA, 12 bp, Homo sapiens
ggaaagctgaga

SEQ ID NO: 347 - DNA, 11 bp, Homo sapiens
ggaaagctgag

SEQ ID NO: 348 - DNA, 10 bp, Homo sapiens
ggaaagctga

SEQ ID NO: 349 - DNA, 42 bp, Homo sapiens
gaaagctgagatggagggcggcatggcgggcacaggctgggc

SEQ ID NO: 350 - DNA, 41 bp, Homo sapiens
gaaagctgagatggagggcggcatggcgggcacaggctggg

SEQ ID NO: 351 - DNA, 40 bp, Homo sapiens
gaaagctgagatggagggcggcatggcgggcacaggctgg

SEQ ID NO: 352 - DNA, 39 bp, Homo sapiens
gaaagctgagatggagggcggcatggcgggcacaggctg

SEQ ID NO: 353 - DNA, 38 bp, Homo sapiens
gaaagctgagatggagggcggcatggcgggcacaggct

SEQ ID NO: 354 - DNA, 37 bp, Homo sapiens
gaaagctgagatggagggcggcatggcgggcacaggc

SEQ ID NO: 355 - DNA, 36 bp, Homo sapiens
gaaagctgagatggagggcggcatggcgggcacagg

SEQ ID NO: 356 - DNA, 35 bp, Homo sapiens
gaaagctgagatggagggcggcatggcgggcacag

SEQ ID NO: 357 - DNA, 34 bp, Homo sapiens
gaaagctgagatggagggcggcatggcgggcaca

SEQ ID NO: 358 - DNA, 33 bp, Homo sapiens
gaaagctgagatggagggcggcatggcgggcac

SEQ ID NO: 359 - DNA, 32 bp, Homo sapiens
gaaagctgagatggagggcggcatggcgggca

SEQ ID NO: 360 - DNA, 31 bp, Homo sapiens
gaaagctgagatggagggcggcatggcgggc

SEQ ID NO: 361 - DNA, 30 bp, Homo sapiens
gaaagctgagatggagggcggcatggcggg

SEQ ID NO: 362 - DNA, 29 bp, Homo sapiens
gaaagctgagatggagggcggcatggcgg

SEQ ID NO: 363 - DNA, 28 bp, Homo sapiens
gaaagctgagatggagggcggcatggcg

SEQ ID NO: 364 - DNA, 27 bp, Homo sapiens
gaaagctgagatggagggcggcatggc

SEQ ID NO: 365 - DNA, 26 bp, Homo sapiens
gaaagctgagatggagggcggcatgg

SEQ ID NO: 366 - DNA, 25 bp, Homo sapiens
gaaagctgagatggagggcggcatg

SEQ ID NO: 367 - DNA, 24 bp, Homo sapiens
gaaagctgagatggagggcggcat

SEQ ID NO: 368 - DNA, 23 bp, Homo sapiens
gaaagctgagatggagggcggca

SEQ ID NO: 369 - DNA, 22 bp, Homo sapiens
gaaagctgagatggagggcggc

SEQ ID NO: 370 - DNA, 21 bp, Homo sapiens
gaaagctgagatggagggcgg

SEQ ID NO: 371 - DNA, 20 bp, Homo sapiens
gaaagctgagatggagggcg

SEQ ID NO: 372 - DNA, 19 bp, Homo sapiens
gaaagctgagatggagggc

SEQ ID NO: 373 - DNA, 18 bp, Homo sapiens
gaaagctgagatggaggg

SEQ ID NO: 374 - DNA, 17 bp, Homo sapiens
gaaagctgagatggagg

SEQ ID NO: 375 - DNA, 16 bp, Homo sapiens
gaaagctgagatggag

SEQ ID NO: 376 - DNA, 15 bp, Homo sapiens
gaaagctgagatgga

SEQ ID NO: 377 - DNA, 14 bp, Homo sapiens
gaaagctgagatgg

SEQ ID NO: 378 - DNA, 13 bp, Homo sapiens
gaaagctgagatg

SEQ ID NO: 379 - DNA, 12 bp, Homo sapiens
gaaagctgagat

SEQ ID NO: 380 - DNA, 11 bp, Homo sapiens
gaaagctgaga

SEQ ID NO: 381 - DNA, 10 bp, Homo sapiens
gaaagctgag

SEQ ID NO: 382 - DNA, 41 bp, Homo sapiens
aaagctgagatggagggcggcatggcgggcacaggctgggc

SEQ ID NO: 383 - DNA, 40 bp, Homo sapiens
aaagctgagatggagggcggcatggcgggcacaggctggg

SEQ ID NO: 384 - DNA, 39 bp, Homo sapiens
aaagctgagatggagggcggcatggcgggcacaggctgg

SEQ ID NO: 385 - DNA, 38 bp, Homo sapiens
aaagctgagatggagggcggcatggcgggcacaggctg

SEQ ID NO: 386 - DNA, 37 bp, Homo sapiens
aaagctgagatggagggcggcatggcgggcacaggct

SEQ ID NO: 387 - DNA, 36 bp, Homo sapiens
aaagctgagatggagggcggcatggcgggcacaggc

SEQ ID NO: 388 - DNA, 35 bp, Homo sapiens
aaagctgagatggagggcggcatggcgggcacagg

SEQ ID NO: 389 - DNA, 34 bp, Homo sapiens
aaagctgagatggagggcggcatggcgggcacag

SEQ ID NO: 390 - DNA, 33 bp, Homo sapiens
aaagctgagatggagggcggcatggcgggcaca

SEQ ID NO: 391 - DNA, 32 bp, Homo sapiens
aaagctgagatggagggcggcatggcgggcac

SEQ ID NO: 392 - DNA, 31 bp, Homo sapiens
aaagctgagatggagggcggcatggcgggca

SEQ ID NO: 393 - DNA, 30 bp, Homo sapiens
aaagctgagatggagggcggcatggcgggc

SEQ ID NO: 394 - DNA, 29 bp, Homo sapiens
aaagctgagatggagggcggcatggcggg

SEQ ID NO: 395 - DNA, 28 bp, Homo sapiens
aaagctgagatggagggcggcatggcgg

SEQ ID NO: 396 - DNA, 27 bp, Homo sapiens
aaagctgagatggagggcggcatggcg

SEQ ID NO: 397 - DNA, 26 bp, Homo sapiens
aaagctgagatggagggcggcatggc

SEQ ID NO: 398 - DNA, 25 bp, Homo sapiens
aaagctgagatggagggcggcatgg

SEQ ID NO: 399 - DNA, 24 bp, Homo sapiens
aaagctgagatggagggcggcatg

SEQ ID NO: 400 - DNA, 23 bp, Homo sapiens
aaagctgagatggagggcggcat

SEQ ID NO: 401 - DNA, 22 bp, Homo sapiens
aaagctgagatggagggcggca

SEQ ID NO: 402 - DNA, 21 bp, Homo sapiens
aaagctgagatggagggcggc

SEQ ID NO: 403 - DNA, 20 bp, Homo sapiens
aaagctgagatggagggcgg

SEQ ID NO: 404 - DNA, 19 bp, Homo sapiens
aaagctgagatggagggcg

SEQ ID NO: 405 - DNA, 18 bp, Homo sapiens
aaagctgagatggagggc

SEQ ID NO: 406 - DNA, 17 bp, Homo sapiens
aaagctgagatggaggg

SEQ ID NO: 407 - DNA, 16 bp, Homo sapiens
aaagctgagatggagg

SEQ ID NO: 408 - DNA, 15 bp, Homo sapiens
aaagctgagatggag

SEQ ID NO: 409 - DNA, 14 bp, Homo sapiens
aaagctgagatgga

SEQ ID NO: 410 - DNA, 13 bp, Homo sapiens
aaagctgagatgg

SEQ ID NO: 411 - DNA, 12 bp, Homo sapiens
aaagctgagatg

SEQ ID NO: 412 - DNA, 11 bp, Homo sapiens
aaagctgagat

SEQ ID NO: 413 - DNA, 10 bp, Homo sapiens
aaagctgaga

SEQ ID NO: 414 - DNA, 40 bp, Homo sapiens
aagctgagatggagggcggcatggcgggcacaggctgggc

SEQ ID NO: 415 - DNA, 39 bp, Homo sapiens
aagctgagatggagggcggcatggcgggcacaggctggg

SEQ ID NO: 416 - DNA, 38 bp, Homo sapiens
aagctgagatggagggcggcatggcgggcacaggctgg

SEQ ID NO: 417 - DNA, 37 bp, Homo sapiens
aagctgagatggagggcggcatggcgggcacaggctg

SEQ ID NO: 418 - DNA, 36 bp, Homo sapiens
aagctgagatggagggcggcatggcgggcacaggct

SEQ ID NO: 419 - DNA, 35 bp, Homo sapiens
aagctgagatggagggcggcatggcgggcacaggc

SEQ ID NO: 420 - DNA, 34 bp, Homo sapiens
aagctgagatggagggcggcatggcgggcacagg

SEQ ID NO: 421 - DNA, 33 bp, Homo sapiens
aagctgagatggagggcggcatggcgggcacag

SEQ ID NO: 422 - DNA, 32 bp, Homo sapiens
aagctgagatggagggcggcatggcgggcaca

SEQ ID NO: 423 - DNA, 31 bp, Homo sapiens
aagctgagatggagggcggcatggcgggcac

SEQ ID NO: 424 - DNA, 30 bp, Homo sapiens
aagctgagatggagggcggcatggcgggca

SEQ ID NO: 425 - DNA, 29 bp, Homo sapiens
aagctgagatggagggcggcatggcgggc

SEQ ID NO: 426 - DNA, 28 bp, Homo sapiens
aagctgagatggagggcggcatggcggg

SEQ ID NO: 427 - DNA, 27 bp, Homo sapiens
aagctgagatggagggcggcatggcgg

SEQ ID NO: 428 - DNA, 26 bp, Homo sapiens
aagctgagatggagggcggcatggcg

SEQ ID NO: 429 - DNA, 25 bp, Homo sapiens
aagctgagatggagggcggcatggc

SEQ ID NO: 430 - DNA, 24 bp, Homo sapiens
aagctgagatggagggcggcatgg

SEQ ID NO: 431 - DNA, 23 bp, Homo sapiens
aagctgagatggagggcggcatg

SEQ ID NO: 432 - DNA, 22 bp, Homo sapiens
aagctgagatggagggcggcat

SEQ ID NO: 433 - DNA, 21 bp, Homo sapiens
aagctgagatggagggcggca

SEQ ID NO: 434 - DNA, 20 bp, Homo sapiens
aagctgagatggagggcggc

SEQ ID NO: 435 - DNA, 19 bp, Homo sapiens
aagctgagatggagggcgg

SEQ ID NO: 436 - DNA, 18 bp, Homo sapiens
aagctgagatggagggcg

SEQ ID NO: 437 - DNA, 17 bp, Homo sapiens
aagctgagatggagggc

SEQ ID NO: 438 - DNA, 16 bp, Homo sapiens
aagctgagatggaggg

SEQ ID NO: 439 - DNA, 15 bp, Homo sapiens
aagctgagatggagg

SEQ ID NO: 440 - DNA, 14 bp, Homo sapiens
aagctgagatggag

SEQ ID NO: 441 - DNA, 13 bp, Homo sapiens
aagctgagatgga

SEQ ID NO: 442 - DNA, 12 bp, Homo sapiens
aagctgagatgg

SEQ ID NO: 443 - DNA, 11 bp, Homo sapiens
aagctgagatg

SEQ ID NO: 444 - DNA, 10 bp, Homo sapiens
aagctgagat

SEQ ID NO: 445 - DNA, 39 bp, Homo sapiens
agctgagatggagggcggcatggcgggcacaggctgggc

SEQ ID NO: 446 - DNA, 38 bp, Homo sapiens
agctgagatggagggcggcatggcgggcacaggctggg

SEQ ID NO: 447 - DNA, 37 bp, Homo sapiens
agctgagatggagggcggcatggcgggcacaggctgg

SEQ ID NO: 448 - DNA, 36 bp, Homo sapiens
agctgagatggagggcggcatggcgggcacaggctg

SEQ ID NO: 449 - DNA, 35 bp, Homo sapiens
agctgagatggagggcggcatggcgggcacaggct

SEQ ID NO: 450 - DNA, 34 bp, Homo sapiens
agctgagatggagggcggcatggcgggcacaggc

SEQ ID NO: 451 - DNA, 33 bp, Homo sapiens
agctgagatggagggcggcatggcgggcacagg

SEQ ID NO: 452 - DNA, 32 bp, Homo sapiens
agctgagatggagggcggcatggcgggcacag

SEQ ID NO: 453 - DNA, 31 bp, Homo sapiens
agctgagatggagggcggcatggcgggcaca

SEQ ID NO: 454 - DNA, 30 bp, Homo sapiens
agctgagatggagggcggcatggcgggcac

SEQ ID NO: 455 - DNA, 29 bp, Homo sapiens
agctgagatggagggcggcatggcgggca

SEQ ID NO: 456 - DNA, 28 bp, Homo sapiens
agctgagatggagggcggcatggcgggc

SEQ ID NO: 457 - DNA, 27 bp, Homo sapiens
agctgagatggagggcggcatggcggg

SEQ ID NO: 458 - DNA, 26 bp, Homo sapiens
agctgagatggagggcggcatggcgg

SEQ ID NO: 459 - DNA, 25 bp, Homo sapiens
agctgagatggagggcggcatggcg

SEQ ID NO: 460 - DNA, 24 bp, Homo sapiens
agctgagatggagggcggcatggc

SEQ ID NO: 461 - DNA, 23 bp, Homo sapiens
agctgagatggagggcggcatgg

SEQ ID NO: 462 - DNA, 22 bp, Homo sapiens
agctgagatggagggcggcatg

SEQ ID NO: 463 - DNA, 21 bp, Homo sapiens
agctgagatggagggcggcat

SEQ ID NO: 464 - DNA, 20 bp, Homo sapiens
agctgagatggagggcggca

SEQ ID NO: 465 - DNA, 19 bp, Homo sapiens
agctgagatggagggcggc

SEQ ID NO: 466 - DNA, 18 bp, Homo sapiens
agctgagatggagggcgg

SEQ ID NO: 467 - DNA, 17 bp, Homo sapiens
agctgagatggagggcg

SEQ ID NO: 468 - DNA, 16 bp, Homo sapiens
agctgagatggagggc

SEQ ID NO: 469 - DNA, 15 bp, Homo sapiens
agctgagatggaggg

SEQ ID NO: 470 - DNA, 14 bp, Homo sapiens
agctgagatggagg

SEQ ID NO: 471 - DNA, 13 bp, Homo sapiens
agctgagatggag

SEQ ID NO: 472 - DNA, 12 bp, Homo sapiens
agctgagatgga

SEQ ID NO: 473 - DNA, 11 bp, Homo sapiens
agctgagatgg

SEQ ID NO: 474 - DNA, 10 bp, Homo sapiens
agctgagatg

SEQ ID NO: 475 - DNA, 38 bp, Homo sapiens
gctgagatggagggcggcatggcgggcacaggctgggc

SEQ ID NO: 476 - DNA, 37 bp, Homo sapiens
gctgagatggagggcggcatggcgggcacaggctggg

SEQ ID NO: 477 - DNA, 36 bp, Homo sapiens
gctgagatggagggcggcatggcgggcacaggctgg

SEQ ID NO: 478 - DNA, 35 bp, Homo sapiens
gctgagatggagggcggcatggcgggcacaggctg

SEQ ID NO: 479 - DNA, 34 bp, Homo sapiens
gctgagatggagggcggcatggcgggcacaggct

SEQ ID NO: 480 - DNA, 33 bp, Homo sapiens
gctgagatggagggcggcatggcgggcacaggc

SEQ ID NO: 481 - DNA, 32 bp, Homo sapiens
gctgagatggagggcggcatggcgggcacagg

SEQ ID NO: 482 - DNA, 31 bp, Homo sapiens
gctgagatggagggcggcatggcgggcacag

SEQ ID NO: 483 - DNA, 30 bp, Homo sapiens
gctgagatggagggcggcatggcgggcaca

SEQ ID NO: 484 - DNA, 29 bp, Homo sapiens
gctgagatggagggcggcatggcgggcac

SEQ ID NO: 485 - DNA, 28 bp, Homo sapiens
gctgagatggagggcggcatggcgggca

SEQ ID NO: 486 - DNA, 27 bp, Homo sapiens
gctgagatggagggcggcatggcgggc

SEQ ID NO: 487 - DNA, 26 bp, Homo sapiens
gctgagatggagggcggcatggcggg

SEQ ID NO: 488 - DNA, 25 bp, Homo sapiens
gctgagatggagggcggcatggcgg

SEQ ID NO: 489 - DNA, 24 bp, Homo sapiens
gctgagatggagggcggcatggcg

SEQ ID NO: 490 - DNA, 23 bp, Homo sapiens
gctgagatggagggcggcatggc

SEQ ID NO: 491 - DNA, 22 bp, Homo sapiens
gctgagatggagggcggcatgg

SEQ ID NO: 492 - DNA, 21 bp, Homo sapiens
gctgagatggagggcggcatg

SEQ ID NO: 493 - DNA, 20 bp, Homo sapiens
gctgagatggagggcggcat

SEQ ID NO: 494 - DNA, 19 bp, Homo sapiens
gctgagatggagggcggca

SEQ ID NO: 495 - DNA, 18 bp, Homo sapiens
gctgagatggagggcggc

SEQ ID NO: 496 - DNA, 17 bp, Homo sapiens
gctgagatggagggcgg

SEQ ID NO: 497 - DNA, 16 bp, Homo sapiens
gctgagatggagggcg

SEQ ID NO: 498 - DNA, 15 bp, Homo sapiens
gctgagatggagggc

SEQ ID NO: 499 - DNA, 14 bp, Homo sapiens
gctgagatggaggg

SEQ ID NO: 500 - DNA, 13 bp, Homo sapiens
gctgagatggagg

SEQ ID NO: 501 - DNA, 12 bp, Homo sapiens
gctgagatggag

SEQ ID NO: 502 - DNA, 11 bp, Homo sapiens
gctgagatgga

SEQ ID NO: 503 - DNA, 10 bp, Homo sapiens
gctgagatgg

SEQ ID NO: 504 - DNA, 37 bp, Homo sapiens
ctgagatggagggcggcatggcgggcacaggctgggc

SEQ ID NO: 505 - DNA, 36 bp, Homo sapiens
ctgagatggagggcggcatggcgggcacaggctggg

SEQ ID NO: 506 - DNA, 35 bp, Homo sapiens
ctgagatggagggcggcatggcgggcacaggctgg

SEQ ID NO: 507 - DNA, 34 bp, Homo sapiens
ctgagatggagggcggcatggcgggcacaggctg

SEQ ID NO: 508 - DNA, 33 bp, Homo sapiens
ctgagatggagggcggcatggcgggcacaggct

SEQ ID NO: 509 - DNA, 32 bp, Homo sapiens
ctgagatggagggcggcatggcgggcacaggc

SEQ ID NO: 510 - DNA, 31 bp, Homo sapiens
ctgagatggagggcggcatggcgggcacagg

SEQ ID NO: 511 - DNA, 30 bp, Homo sapiens
ctgagatggagggcggcatggcgggcacag

SEQ ID NO: 512 - DNA, 29 bp, Homo sapiens
ctgagatggagggcggcatggcgggcaca

SEQ ID NO: 513 - DNA, 28 bp, Homo sapiens
ctgagatggagggcggcatggcgggcac

SEQ ID NO: 514 - DNA, 27 bp, Homo sapiens
ctgagatggagggcggcatggcgggca

SEQ ID NO: 515 - DNA, 26 bp, Homo sapiens
ctgagatggagggcggcatggcgggc

SEQ ID NO: 516 - DNA, 25 bp, Homo sapiens
ctgagatggagggcggcatggcggg

SEQ ID NO: 517 - DNA, 24 bp, Homo sapiens
ctgagatggagggcggcatggcgg

SEQ ID NO: 518 - DNA, 23 bp, Homo sapiens
ctgagatggagggcggcatggcg

SEQ ID NO: 519 - DNA, 22 bp, Homo sapiens
ctgagatggagggcggcatggc

SEQ ID NO: 520 - DNA, 21 bp, Homo sapiens
ctgagatggagggcggcatgg

SEQ ID NO: 521 - DNA, 20 bp, Homo sapiens
ctgagatggagggcggcatg

SEQ ID NO: 522 - DNA, 19 bp, Homo sapiens
ctgagatggagggcggcat

SEQ ID NO: 523 - DNA, 18 bp, Homo sapiens
ctgagatggagggcggca

SEQ ID NO: 524 - DNA, 17 bp, Homo sapiens
ctgagatggagggcggc

SEQ ID NO: 525 - DNA, 16 bp, Homo sapiens
ctgagatggagggcgg

SEQ ID NO: 526 - DNA, 15 bp, Homo sapiens
ctgagatggagggcg

SEQ ID NO: 527 - DNA, 14 bp, Homo sapiens
ctgagatggagggc

SEQ ID NO: 528 - DNA, 13 bp, Homo sapiens
ctgagatggaggg

SEQ ID NO: 529 - DNA, 12 bp, Homo sapiens
ctgagatggagg

SEQ ID NO: 530 - DNA, 11 bp, Homo sapiens
ctgagatggag

SEQ ID NO: 531 - DNA, 10 bp, Homo sapiens
ctgagatgga

SEQ ID NO: 532 - DNA, 36 bp, Homo sapiens
tgagatggagggcggcatggcgggcacaggctgggc

SEQ ID NO: 533 - DNA, 35 bp, Homo sapiens
tgagatggagggcggcatggcgggcacaggctggg

SEQ ID NO: 534 - DNA, 34 bp, Homo sapiens
tgagatggagggcggcatggcgggcacaggctgg

SEQ ID NO: 535 - DNA, 33 bp, Homo sapiens
tgagatggagggcggcatggcgggcacaggctg

SEQ ID NO: 536 - DNA, 32 bp, Homo sapiens
tgagatggagggcggcatggcgggcacaggct

SEQ ID NO: 537 - DNA, 31 bp, Homo sapiens
tgagatggagggcggcatggcgggcacaggc

SEQ ID NO: 538 - DNA, 30 bp, Homo sapiens
tgagatggagggcggcatggcgggcacagg

SEQ ID NO: 539 - DNA, 29 bp, Homo sapiens
tgagatggagggcggcatggcgggcacag

SEQ ID NO: 540 - DNA, 28 bp, Homo sapiens
tgagatggagggcggcatggcgggcaca

SEQ ID NO: 541 - DNA, 27 bp, Homo sapiens
tgagatggagggcggcatggcgggcac

SEQ ID NO: 542 - DNA, 26 bp, Homo sapiens
tgagatggagggcggcatggcgggca

SEQ ID NO: 543 - DNA, 25 bp, Homo sapiens
tgagatggagggcggcatggcgggc

SEQ ID NO: 544 - DNA, 24 bp, Homo sapiens
tgagatggagggcggcatggcggg

SEQ ID NO: 545 - DNA, 23 bp, Homo sapiens
tgagatggagggcggcatggcgg

SEQ ID NO: 546 - DNA, 22 bp, Homo sapiens
tgagatggagggcggcatggcg

SEQ ID NO: 547 - DNA, 21 bp, Homo sapiens
tgagatggagggcggcatggc

SEQ ID NO: 548 - DNA, 20 bp, Homo sapiens
tgagatggagggcggcatgg

SEQ ID NO: 549 - DNA, 19 bp, Homo sapiens
tgagatggagggcggcatg

SEQ ID NO: 550 - DNA, 18 bp, Homo sapiens
tgagatggagggcggcat

SEQ ID NO: 551 - DNA, 17 bp, Homo sapiens
tgagatggagggcggca

SEQ ID NO: 552 - DNA, 16 bp, Homo sapiens
tgagatggagggcggc

SEQ ID NO: 553 - DNA, 15 bp, Homo sapiens
tgagatggagggcgg

SEQ ID NO: 554 - DNA, 14 bp, Homo sapiens
tgagatggagggcg

SEQ ID NO: 555 - DNA, 13 bp, Homo sapiens
tgagatggagggc

SEQ ID NO: 556 - DNA, 12 bp, Homo sapiens
tgagatggaggg

SEQ ID NO: 557 - DNA, 11 bp, Homo sapiens
tgagatggagg

SEQ ID NO: 558 - DNA, 10 bp, Homo sapiens
tgagatggag

SEQ ID NO: 559 - DNA, 35 bp, Homo sapiens
gagatggagggcggcatggcgggcacaggctgggc

SEQ ID NO: 560 - DNA, 34 bp, Homo sapiens
gagatggagggcggcatggcgggcacaggctggg

SEQ ID NO: 561 - DNA, 33 bp, Homo sapiens
gagatggagggcggcatggcgggcacaggctgg

SEQ ID NO: 562 - DNA, 32 bp, Homo sapiens
gagatggagggcggcatggcgggcacaggctg

SEQ ID NO: 563 - DNA, 31 bp, Homo sapiens
gagatggagggcggcatggcgggcacaggct

SEQ ID NO: 564 - DNA, 30 bp, Homo sapiens
gagatggagggcggcatggcgggcacaggc

SEQ ID NO: 565 - DNA, 29 bp, Homo sapiens
gagatggagggcggcatggcgggcacagg

SEQ ID NO: 566 - DNA, 28 bp, Homo sapiens
gagatggagggcggcatggcgggcacag

SEQ ID NO: 567 - DNA, 27 bp, Homo sapiens
gagatggagggcggcatggcgggcaca

SEQ ID NO: 568 - DNA, 26 bp, Homo sapiens
gagatggagggcggcatggcgggcac

SEQ ID NO: 569 - DNA, 25 bp, Homo sapiens
gagatggagggcggcatggcgggca

SEQ ID NO: 570 - DNA, 24 bp, Homo sapiens
gagatggagggcggcatggcgggc

SEQ ID NO: 571 - DNA, 23 bp, Homo sapiens
gagatggagggcggcatggcggg

SEQ ID NO: 572 - DNA, 22 bp, Homo sapiens
gagatggagggcggcatggcgg

SEQ ID NO: 573 - DNA, 21 bp, Homo sapiens
gagatggagggcggcatggcg

SEQ ID NO: 574 - DNA, 20 bp, Homo sapiens
gagatggagggcggcatggc

SEQ ID NO: 575 - DNA, 19 bp, Homo sapiens
gagatggagggcggcatgg

SEQ ID NO: 576 - DNA, 18 bp, Homo sapiens
gagatggagggcggcatg

SEQ ID NO: 577 - DNA, 17 bp, Homo sapiens
gagatggagggcggcat

SEQ ID NO: 578 - DNA, 16 bp, Homo sapiens
gagatggagggcggca

SEQ ID NO: 579 - DNA, 15 bp, Homo sapiens
gagatggagggcggc

SEQ ID NO: 580 - DNA, 14 bp, Homo sapiens
gagatggagggcgg

SEQ ID NO: 581 - DNA, 13 bp, Homo sapiens
gagatggagggcg

SEQ ID NO: 582 - DNA, 12 bp, Homo sapiens
gagatggagggc

SEQ ID NO: 583 - DNA, 11 bp, Homo sapiens
gagatggaggg

SEQ ID NO: 584 - DNA, 10 bp, Homo sapiens
gagatggagg

SEQ ID NO: 585 - DNA, 34 bp, Homo sapiens
agatggagggcggcatggcgggcacaggctgggc

SEQ ID NO: 586 - DNA, 33 bp, Homo sapiens
agatggagggcggcatggcgggcacaggctggg

SEQ ID NO: 587 - DNA, 32 bp, Homo sapiens
agatggagggcggcatggcgggcacaggctgg

SEQ ID NO: 588 - DNA, 31 bp, Homo sapiens
agatggagggcggcatggcgggcacaggctg

SEQ ID NO: 589 - DNA, 30 bp, Homo sapiens
agatggagggcggcatggcgggcacaggct

SEQ ID NO: 590 - DNA, 29 bp, Homo sapiens
agatggagggcggcatggcgggcacaggc

SEQ ID NO: 591 - DNA, 28 bp, Homo sapiens
agatggagggcggcatggcgggcacagg

SEQ ID NO: 592 - DNA, 27 bp, Homo sapiens
agatggagggcggcatggcgggcacag

SEQ ID NO: 593 - DNA, 26 bp, Homo sapiens
agatggagggcggcatggcgggcaca

SEQ ID NO: 594 - DNA, 25 bp, Homo sapiens
agatggagggcggcatggcgggcac

SEQ ID NO: 595 - DNA, 24 bp, Homo sapiens
agatggagggcggcatggcgggca

SEQ ID NO: 596 - DNA, 23 bp, Homo sapiens
agatggagggcggcatggcgggc

SEQ ID NO: 597 - DNA, 22 bp, Homo sapiens
agatggagggcggcatggcggg

SEQ ID NO: 598 - DNA, 21 bp, Homo sapiens
agatggagggcggcatggcgg

SEQ ID NO: 599 - DNA, 20 bp, Homo sapiens
agatggagggcggcatggcg

SEQ ID NO: 600 - DNA, 19 bp, Homo sapiens
agatggagggcggcatggc

SEQ ID NO: 601 - DNA, 18 bp, Homo sapiens
agatggagggcggcatgg

SEQ ID NO: 602 - DNA, 17 bp, Homo sapiens
agatggagggcggcatg

SEQ ID NO: 603 - DNA, 16 bp, Homo sapiens
agatggagggcggcat

SEQ ID NO: 604 - DNA, 15 bp, Homo sapiens
agatggagggcggca

SEQ ID NO: 605 - DNA, 14 bp, Homo sapiens
agatggagggcggc

SEQ ID NO: 606 - DNA, 13 bp, Homo sapiens
agatggagggcgg

SEQ ID NO: 607 - DNA, 12 bp, Homo sapiens
agatggagggcg

SEQ ID NO: 608 - DNA, 11 bp, Homo sapiens
agatggagggc

SEQ ID NO: 609 - DNA, 10 bp, Homo sapiens
agatggaggg

SEQ ID NO: 610 - DNA, 33 bp, Homo sapiens
gatggagggcggcatggcgggcacaggctgggc

SEQ ID NO: 611 - DNA, 32 bp, Homo sapiens
gatggagggcggcatggcgggcacaggctggg

SEQ ID NO: 612 - DNA, 31 bp, Homo sapiens
gatggagggcggcatggcgggcacaggctgg

SEQ ID NO: 613 - DNA, 30 bp, Homo sapiens
gatggagggcggcatggcgggcacaggctg

SEQ ID NO: 614 - DNA, 29 bp, Homo sapiens
gatggagggcggcatggcgggcacaggct

SEQ ID NO: 615 - DNA, 28 bp, Homo sapiens
gatggagggcggcatggcgggcacaggc

SEQ ID NO: 616 - DNA, 27 bp, Homo sapiens
gatggagggcggcatggcgggcacagg

SEQ ID NO: 617 - DNA, 26 bp, Homo sapiens
gatggagggcggcatggcgggcacag

SEQ ID NO: 618 - DNA, 25 bp, Homo sapiens
gatggagggcggcatggcgggcaca

SEQ ID NO: 619 - DNA, 24 bp, Homo sapiens
gatggagggcggcatggcgggcac

SEQ ID NO: 620 - DNA, 23 bp, Homo sapiens
gatggagggcggcatggcgggca

SEQ ID NO: 621 - DNA, 22 bp, Homo sapiens
gatggagggcggcatggcgggc

SEQ ID NO: 622 - DNA, 21 bp, Homo sapiens
gatggagggcggcatggcggg

SEQ ID NO: 623 - DNA, 20 bp, Homo sapiens
gatggagggcggcatggcgg

SEQ ID NO: 624 - DNA, 19 bp, Homo sapiens
gatggagggcggcatggcg

SEQ ID NO: 625 - DNA, 18 bp, Homo sapiens
gatggagggcggcatggc

SEQ ID NO: 626 - DNA, 17 bp, Homo sapiens
gatggagggcggcatgg

SEQ ID NO: 627 - DNA, 16 bp, Homo sapiens
gatggagggcggcatg

SEQ ID NO: 628 - DNA, 15 bp, Homo sapiens
gatggagggcggcat

SEQ ID NO: 629 - DNA, 14 bp, Homo sapiens
gatggagggcggca

SEQ ID NO: 630 - DNA, 13 bp, Homo sapiens
gatggagggcggc

SEQ ID NO: 631 - DNA, 12 bp, Homo sapiens
gatggagggcgg

SEQ ID NO: 632 - DNA, 11 bp, Homo sapiens
gatggagggcg

SEQ ID NO: 633 - DNA, 10 bp, Homo sapiens
gatggagggc

SEQ ID NO: 634 - DNA, 32 bp, Homo sapiens
atggagggcggcatggcgggcacaggctgggc

SEQ ID NO: 635 - DNA, 31 bp, Homo sapiens
atggagggcggcatggcgggcacaggctggg

SEQ ID NO: 636 - DNA, 30 bp, Homo sapiens
atggagggcggcatggcgggcacaggctgg

SEQ ID NO: 637 - DNA, 29 bp, Homo sapiens
atggagggcggcatggcgggcacaggctg

SEQ ID NO: 638 - DNA, 28 bp, Homo sapiens
atggagggcggcatggcgggcacaggct

SEQ ID NO: 639 - DNA, 27 bp, Homo sapiens
atggagggcggcatggcgggcacaggc

SEQ ID NO: 640 - DNA, 26 bp, Homo sapiens
atggagggcggcatggcgggcacagg

SEQ ID NO: 641 - DNA, 25 bp, Homo sapiens
atggagggcggcatggcgggcacag

SEQ ID NO: 642 - DNA, 24 bp, Homo sapiens
atggagggcggcatggcgggcaca

SEQ ID NO: 643 - DNA, 23 bp, Homo sapiens
atggagggcggcatggcgggcac

SEQ ID NO: 644 - DNA, 22 bp, Homo sapiens
atggagggcggcatggcgggca

SEQ ID NO: 645 - DNA, 21 bp, Homo sapiens
atggagggcggcatggcgggc

SEQ ID NO: 646 - DNA, 20 bp, Homo sapiens
atggagggcggcatggcggg

SEQ ID NO: 647 - DNA, 19 bp, Homo sapiens
atggagggcggcatggcgg

SEQ ID NO: 648 - DNA, 18 bp, Homo sapiens
atggagggcggcatggcg

SEQ ID NO: 649 - DNA, 17 bp, Homo sapiens
atggagggcggcatggc

SEQ ID NO: 650 - DNA, 16 bp, Homo sapiens
atggagggcggcatgg

SEQ ID NO: 651 - DNA, 15 bp, Homo sapiens
atggagggcggcatg

SEQ ID NO: 652 - DNA, 14 bp, Homo sapiens
atggagggcggcat

SEQ ID NO: 653 - DNA, 13 bp, Homo sapiens
atggagggcggca

SEQ ID NO: 654 - DNA, 12 bp, Homo sapiens
atggagggcggc

SEQ ID NO: 655 - DNA, 11 bp, Homo sapiens
atggagggcgg

SEQ ID NO: 656 - DNA, 10 bp, Homo sapiens
atggagggcg

SEQ ID NO: 657 - DNA, 31 bp, Homo sapiens
tggagggcggcatggcgggcacaggctgggc

SEQ ID NO: 658 - DNA, 30 bp, Homo sapiens
tggagggcggcatggcgggcacaggctggg

SEQ ID NO: 659 - DNA, 29 bp, Homo sapiens
tggagggcggcatggcgggcacaggctgg

SEQ ID NO: 660 - DNA, 28 bp, Homo sapiens
tggagggcggcatggcgggcacaggctg

SEQ ID NO: 661 - DNA, 27 bp, Homo sapiens
tggagggcggcatggcgggcacaggct

SEQ ID NO: 662 - DNA, 26 bp, Homo sapiens
tggagggcggcatggcgggcacaggc

SEQ ID NO: 663 - DNA, 25 bp, Homo sapiens
tggagggcggcatggcgggcacagg

SEQ ID NO: 664 - DNA, 24 bp, Homo sapiens
tggagggcggcatggcgggcacag

SEQ ID NO: 665 - DNA, 23 bp, Homo sapiens
tggagggcggcatggcgggcaca

SEQ ID NO: 666 - DNA, 22 bp, Homo sapiens
tggagggcggcatggcgggcac

SEQ ID NO: 667 - DNA, 21 bp, Homo sapiens
tggagggcggcatggcgggca

SEQ ID NO: 668 - DNA, 20 bp, Homo sapiens
tggagggcggcatggcgggc

SEQ ID NO: 669 - DNA, 19 bp, Homo sapiens
tggagggcggcatggcggg

SEQ ID NO: 670 - DNA, 18 bp, Homo sapiens
tggagggcggcatggcgg

SEQ ID NO: 671 - DNA, 17 bp, Homo sapiens
tggagggcggcatggcg

SEQ ID NO: 672 - DNA, 16 bp, Homo sapiens
tggagggcggcatggc

SEQ ID NO: 673 - DNA, 15 bp, Homo sapiens
tggagggcggcatgg

SEQ ID NO: 674 - DNA, 14 bp, Homo sapiens
tggagggcggcatg

SEQ ID NO: 675 - DNA, 13 bp, Homo sapiens
tggagggcggcat

SEQ ID NO: 676 - DNA, 12 bp, Homo sapiens
tggagggcggca

SEQ ID NO: 677 - DNA, 11 bp, Homo sapiens
tggagggcggc

SEQ ID NO: 678 - DNA, 10 bp, Homo sapiens
tggagggcgg

SEQ ID NO: 679 - DNA, 30 bp, Homo sapiens
ggagggcggcatggcgggcacaggctgggc

SEQ ID NO: 680 - DNA, 29 bp, Homo sapiens
ggagggcggcatggcgggcacaggctggg

SEQ ID NO: 681 - DNA, 28 bp, Homo sapiens
ggagggcggcatggcgggcacaggctgg

SEQ ID NO: 682 - DNA, 27 bp, Homo sapiens
ggagggcggcatggcgggcacaggctg

SEQ ID NO: 683 - DNA, 26 bp, Homo sapiens
ggagggcggcatggcgggcacaggct

SEQ ID NO: 684 - DNA, 25 bp, Homo sapiens
ggagggcggcatggcgggcacaggc

SEQ ID NO: 685 - DNA, 24 bp, Homo sapiens
ggagggcggcatggcgggcacagg

SEQ ID NO: 686 - DNA, 23 bp, Homo sapiens
ggagggcggcatggcgggcacag

SEQ ID NO: 687 - DNA, 22 bp, Homo sapiens
ggagggcggcatggcgggcaca

SEQ ID NO: 688 - DNA, 21 bp, Homo sapiens
ggagggcggcatggcgggcac

SEQ ID NO: 689 - DNA, 20 bp, Homo sapiens
ggagggcggcatggcgggca

SEQ ID NO: 690 - DNA, 19 bp, Homo sapiens
ggagggcggcatggcgggc

SEQ ID NO: 691 - DNA, 18 bp, Homo sapiens
ggagggcggcatggcggg

SEQ ID NO: 692 - DNA, 17 bp, Homo sapiens
ggagggcggcatggcgg

SEQ ID NO: 693 - DNA, 16 bp, Homo sapiens
ggagggcggcatggcg

SEQ ID NO: 694 - DNA, 15 bp, Homo sapiens
ggagggcggcatggc

SEQ ID NO: 695 - DNA, 14 bp, Homo sapiens
ggagggcggcatgg

SEQ ID NO: 696 - DNA, 13 bp, Homo sapiens
ggagggcggcatg

SEQ ID NO: 697 - DNA, 12 bp, Homo sapiens
ggagggcggcat

SEQ ID NO: 698 - DNA, 11 bp, Homo sapiens
ggagggcggca

SEQ ID NO: 699 - DNA, 10 bp, Homo sapiens
ggagggcggc

SEQ ID NO: 700 - DNA, 29 bp, Homo sapiens
gagggcggcatggcgggcacaggctgggc

SEQ ID NO: 701 - DNA, 28 bp, Homo sapiens
gagggcggcatggcgggcacaggctggg

SEQ ID NO: 702 - DNA, 27 bp, Homo sapiens
gagggcggcatggcgggcacaggctgg

SEQ ID NO: 703 - DNA, 26 bp, Homo sapiens
gagggcggcatggcgggcacaggctg

SEQ ID NO: 704 - DNA, 25 bp, Homo sapiens
gagggcggcatggcgggcacaggct

SEQ ID NO: 705 - DNA, 24 bp, Homo sapiens
gagggcggcatggcgggcacaggc

SEQ ID NO: 706 - DNA, 23 bp, Homo sapiens
gagggcggcatggcgggcacagg

SEQ ID NO: 707 - DNA, 22 bp, Homo sapiens
gagggcggcatggcgggcacag

SEQ ID NO: 708 - DNA, 21 bp, Homo sapiens
gagggcggcatggcgggcaca

SEQ ID NO: 709 - DNA, 20 bp, Homo sapiens
gagggcggcatggcgggcac

SEQ ID NO: 710 - DNA, 19 bp, Homo sapiens
gagggcggcatggcgggca

SEQ ID NO: 711 - DNA, 18 bp, Homo sapiens
gagggcggcatggcgggc

SEQ ID NO: 712 - DNA, 17 bp, Homo sapiens
gagggcggcatggcggg

SEQ ID NO: 713 - DNA, 16 bp, Homo sapiens
gagggcggcatggcgg

SEQ ID NO: 714 - DNA, 15 bp, Homo sapiens
gagggcggcatggcg

SEQ ID NO: 715 - DNA, 14 bp, Homo sapiens
gagggcggcatggc

SEQ ID NO: 716 - DNA, 13 bp, Homo sapiens
gagggcggcatgg

SEQ ID NO: 717 - DNA, 12 bp, Homo sapiens
gagggcggcatg

SEQ ID NO: 718 - DNA, 11 bp, Homo sapiens
gagggcggcat

SEQ ID NO: 719 - DNA, 10 bp, Homo sapiens
gagggcggca

SEQ ID NO: 720 - DNA, 28 bp, Homo sapiens
agggcggcatggcgggcacaggctgggc

SEQ ID NO: 721 - DNA, 27 bp, Homo sapiens
agggcggcatggcgggcacaggctggg

SEQ ID NO: 722 - DNA, 26 bp, Homo sapiens
agggcggcatggcgggcacaggctgg

SEQ ID NO: 723 - DNA, 25 bp, Homo sapiens
agggcggcatggcgggcacaggctg

SEQ ID NO: 724 - DNA, 24 bp, Homo sapiens
agggcggcatggcgggcacaggct

SEQ ID NO: 725 - DNA, 23 bp, Homo sapiens
agggcggcatggcgggcacaggc

SEQ ID NO: 726 - DNA, 22 bp, Homo sapiens
agggcggcatggcgggcacagg

SEQ ID NO: 727 - DNA, 21 bp, Homo sapiens
agggcggcatggcgggcacag

SEQ ID NO: 728 - DNA, 20 bp, Homo sapiens
agggcggcatggcgggcaca

SEQ ID NO: 729 - DNA, 19 bp, Homo sapiens
agggcggcatggcgggcac

SEQ ID NO: 730 - DNA, 18 bp, Homo sapiens
agggcggcatggcgggca

SEQ ID NO: 731 - DNA, 17 bp, Homo sapiens
agggcggcatggcgggc

SEQ ID NO: 732 - DNA, 16 bp, Homo sapiens
agggcggcatggcggg

SEQ ID NO: 733 - DNA, 15 bp, Homo sapiens
agggcggcatggcgg

SEQ ID NO: 734 - DNA, 14 bp, Homo sapiens
agggcggcatggcg

SEQ ID NO: 735 - DNA, 13 bp, Homo sapiens
agggcggcatggc

SEQ ID NO: 736 - DNA, 12 bp, Homo sapiens
agggcggcatgg

SEQ ID NO: 737 - DNA, 11 bp, Homo sapiens
agggcggcatg

SEQ ID NO: 738 - DNA, 10 bp, Homo sapiens
agggcggcat

SEQ ID NO: 739 - DNA, 27 bp, Homo sapiens
gggcggcatggcgggcacaggctgggc

SEQ ID NO: 740 - DNA, 26 bp, Homo sapiens
gggcggcatggcgggcacaggctggg

SEQ ID NO: 741 - DNA, 25 bp, Homo sapiens
gggcggcatggcgggcacaggctgg

SEQ ID NO: 742 - DNA, 24 bp, Homo sapiens
gggcggcatggcgggcacaggctg

SEQ ID NO: 743 - DNA, 23 bp, Homo sapiens
gggcggcatggcgggcacaggct

SEQ ID NO: 744 - DNA, 22 bp, Homo sapiens
gggcggcatggcgggcacaggc

SEQ ID NO: 745 - DNA, 21 bp, Homo sapiens
gggcggcatggcgggcacagg

SEQ ID NO: 746 - DNA, 20 bp, Homo sapiens
gggcggcatggcgggcacag

SEQ ID NO: 747 - DNA, 19 bp, Homo sapiens
gggcggcatggcgggcaca

SEQ ID NO: 748 - DNA, 18 bp, Homo sapiens
gggcggcatggcgggcac

SEQ ID NO: 749 - DNA, 17 bp, Homo sapiens
gggcggcatggcgggca

SEQ ID NO: 750 - DNA, 16 bp, Homo sapiens
gggcggcatggcgggc

SEQ ID NO: 751 - DNA, 15 bp, Homo sapiens
gggcggcatggcggg

SEQ ID NO: 752 - DNA, 14 bp, Homo sapiens
gggcggcatggcgg

SEQ ID NO: 753 - DNA, 13 bp, Homo sapiens
gggcggcatggcg

SEQ ID NO: 754 - DNA, 12 bp, Homo sapiens
gggcggcatggc

SEQ ID NO: 755 - DNA, 11 bp, Homo sapiens
gggcggcatgg

SEQ ID NO: 756 - DNA, 10 bp, Homo sapiens
gggcggcatg

SEQ ID NO: 757 - DNA, 26 bp, Homo sapiens
ggcggcatggcgggcacaggctgggc

SEQ ID NO: 758 - DNA, 25 bp, Homo sapiens
ggcggcatggcgggcacaggctggg

SEQ ID NO: 759 - DNA, 24 bp, Homo sapiens
ggcggcatggcgggcacaggctgg

SEQ ID NO: 760 - DNA, 23 bp, Homo sapiens
ggcggcatggcgggcacaggctg

SEQ ID NO: 761 - DNA, 22 bp, Homo sapiens
ggcggcatggcgggcacaggct

SEQ ID NO: 762 - DNA, 21 bp, Homo sapiens
ggcggcatggcgggcacaggc

SEQ ID NO: 763 - DNA, 20 bp, Homo sapiens
ggcggcatggcgggcacagg

SEQ ID NO: 764 - DNA, 19 bp, Homo sapiens
ggcggcatggcgggcacag

SEQ ID NO: 765 - DNA, 18 bp, Homo sapiens
ggcggcatggcgggcaca

SEQ ID NO: 766 - DNA, 17 bp, Homo sapiens
ggcggcatggcgggcac

SEQ ID NO: 767 - DNA, 16 bp, Homo sapiens
ggcggcatggcgggca

SEQ ID NO: 768 - DNA, 15 bp, Homo sapiens
ggcggcatggcgggc

SEQ ID NO: 769 - DNA, 14 bp, Homo sapiens
ggcggcatggcggg

SEQ ID NO: 770 - DNA, 13 bp, Homo sapiens
ggcggcatggcgg

SEQ ID NO: 771 - DNA, 12 bp, Homo sapiens
ggcggcatggcg

SEQ ID NO: 772 - DNA, 11 bp, Homo sapiens
ggcggcatggc

SEQ ID NO: 773 - DNA, 10 bp, Homo sapiens
ggcggcatgg

SEQ ID NO: 774 - DNA, 25 bp, Homo sapiens
gcggcatggcgggcacaggctgggc

SEQ ID NO: 775 - DNA, 24 bp, Homo sapiens
gcggcatggcgggcacaggctggg

SEQ ID NO: 776 - DNA, 23 bp, Homo sapiens
gcggcatggcgggcacaggctgg

SEQ ID NO: 777 - DNA, 22 bp, Homo sapiens
gcggcatggcgggcacaggctg

SEQ ID NO: 778 - DNA, 21 bp, Homo sapiens
gcggcatggcgggcacaggct

SEQ ID NO: 779 - DNA, 20 bp, Homo sapiens
gcggcatggcgggcacaggc

SEQ ID NO: 780 - DNA, 19 bp, Homo sapiens
gcggcatggcgggcacagg

SEQ ID NO: 781 - DNA, 18 bp, Homo sapiens
gcggcatggcgggcacag

SEQ ID NO: 782 - DNA, 17 bp, Homo sapiens
gcggcatggcgggcaca

SEQ ID NO: 783 - DNA, 16 bp, Homo sapiens
gcggcatggcgggcac

SEQ ID NO: 784 - DNA, 15 bp, Homo sapiens
gcggcatggcgggca

SEQ ID NO: 785 - DNA, 14 bp, Homo sapiens
gcggcatggcgggc

SEQ ID NO: 786 - DNA, 13 bp, Homo sapiens
gcggcatggcggg

SEQ ID NO: 787 - DNA, 12 bp, Homo sapiens
gcggcatggcgg

SEQ ID NO: 788 - DNA, 11 bp, Homo sapiens
gcggcatggcg

SEQ ID NO: 789 - DNA, 10 bp, Homo sapiens
gcggcatggc

SEQ ID NO: 790 - DNA, 24 bp, Homo sapiens
cggcatggcgggcacaggctgggc

SEQ ID NO: 791 - DNA, 23 bp, Homo sapiens
cggcatggcgggcacaggctggg

SEQ ID NO: 792 - DNA, 22 bp, Homo sapiens
cggcatggcgggcacaggctgg

SEQ ID NO: 793 - DNA, 21 bp, Homo sapiens
cggcatggcgggcacaggctg

SEQ ID NO: 794 - DNA, 20 bp, Homo sapiens
cggcatggcgggcacaggct

SEQ ID NO: 795 - DNA, 19 bp, Homo sapiens
cggcatggcgggcacaggc

SEQ ID NO: 796 - DNA, 18 bp, Homo sapiens
cggcatggcgggcacagg

SEQ ID NO: 797 - DNA, 17 bp, Homo sapiens
cggcatggcgggcacag

SEQ ID NO: 798 - DNA, 16 bp, Homo sapiens
cggcatggcgggcaca

SEQ ID NO: 799 - DNA, 15 bp, Homo sapiens
cggcatggcgggcac

SEQ ID NO: 800 - DNA, 14 bp, Homo sapiens
cggcatggcgggca

SEQ ID NO: 801 - DNA, 13 bp, Homo sapiens
cggcatggcgggc

SEQ ID NO: 802 - DNA, 12 bp, Homo sapiens
cggcatggcggg

SEQ ID NO: 803 - DNA, 11 bp, Homo sapiens
cggcatggcgg

SEQ ID NO: 804 - DNA, 10 bp, Homo sapiens
cggcatggcg

SEQ ID NO: 805 - DNA, 23 bp, Homo sapiens
ggcatggcgggcacaggctgggc

SEQ ID NO: 806 - DNA, 22 bp, Homo sapiens
ggcatggcgggcacaggctggg

SEQ ID NO: 807 - DNA, 21 bp, Homo sapiens
ggcatggcgggcacaggctgg

SEQ ID NO: 808 - DNA, 20 bp, Homo sapiens
ggcatggcgggcacaggctg

SEQ ID NO: 809 - DNA, 19 bp, Homo sapiens
ggcatggcgggcacaggct

SEQ ID NO: 810 - DNA, 18 bp, Homo sapiens
ggcatggcgggcacaggc

SEQ ID NO: 811 - DNA, 17 bp, Homo sapiens
ggcatggcgggcacagg

SEQ ID NO: 812 - DNA, 16 bp, Homo sapiens
ggcatggcgggcacag

SEQ ID NO: 813 - DNA, 15 bp, Homo sapiens
ggcatggcgggcaca

SEQ ID NO: 814 - DNA, 14 bp, Homo sapiens
ggcatggcgggcac

SEQ ID NO: 815 - DNA, 13 bp, Homo sapiens
ggcatggcgggca

SEQ ID NO: 816 - DNA, 12 bp, Homo sapiens
ggcatggcgggc

SEQ ID NO: 817 - DNA, 11 bp, Homo sapiens
ggcatggcggg

SEQ ID NO: 818 - DNA, 10 bp, Homo sapiens
ggcatggcgg

SEQ ID NO: 819 - DNA, 22 bp, Homo sapiens
gcatggcgggcacaggctgggc

SEQ ID NO: 820 - DNA, 21 bp, Homo sapiens
gcatggcgggcacaggctggg

SEQ ID NO: 821 - DNA, 20 bp, Homo sapiens
gcatggcgggcacaggctgg

SEQ ID NO: 822 - DNA, 19 bp, Homo sapiens
gcatggcgggcacaggctg

SEQ ID NO: 823 - DNA, 18 bp, Homo sapiens
gcatggcgggcacaggct

SEQ ID NO: 824 - DNA, 17 bp, Homo sapiens
gcatggcgggcacaggc

SEQ ID NO: 825 - DNA, 16 bp, Homo sapiens
gcatggcgggcacagg

SEQ ID NO: 826 - DNA, 15 bp, Homo sapiens
gcatggcgggcacag

SEQ ID NO: 827 - DNA, 14 bp, Homo sapiens
gcatggcgggcaca

SEQ ID NO: 828 - DNA, 13 bp, Homo sapiens
gcatggcgggcac

SEQ ID NO: 829 - DNA, 12 bp, Homo sapiens
gcatggcgggca

SEQ ID NO: 830 - DNA, 11 bp, Homo sapiens
gcatggcgggc

SEQ ID NO: 831 - DNA, 10 bp, Homo sapiens
gcatggcggg

SEQ ID NO: 832 - DNA, 21 bp, Homo sapiens
catggcgggcacaggctgggc

SEQ ID NO: 833 - DNA, 20 bp, Homo sapiens
catggcgggcacaggctggg

SEQ ID NO: 834 - DNA, 19 bp, Homo sapiens
catggcgggcacaggctgg

SEQ ID NO: 835 - DNA, 18 bp, Homo sapiens
catggcgggcacaggctg

SEQ ID NO: 836 - DNA, 17 bp, Homo sapiens
catggcgggcacaggct

SEQ ID NO: 837 - DNA, 16 bp, Homo sapiens
catggcgggcacaggc

SEQ ID NO: 838 - DNA, 15 bp, Homo sapiens
catggcgggcacagg

SEQ ID NO: 839 - DNA, 14 bp, Homo sapiens
catggcgggcacag

SEQ ID NO: 840 - DNA, 13 bp, Homo sapiens
catggcgggcaca

SEQ ID NO: 841 - DNA, 12 bp, Homo sapiens
catggcgggcac

SEQ ID NO: 842 - DNA, 11 bp, Homo sapiens
catggcgggca

SEQ ID NO: 843 - DNA, 10 bp, Homo sapiens
catggcgggc

SEQ ID NO: 844 - DNA, 20 bp, Homo sapiens
atggcgggcacaggctgggc

SEQ ID NO: 845 - DNA, 19 bp, Homo sapiens
atggcgggcacaggctggg

SEQ ID NO: 846 - DNA, 18 bp, Homo sapiens
atggcgggcacaggctgg

SEQ ID NO: 847 - DNA, 17 bp, Homo sapiens
atggcgggcacaggctg

SEQ ID NO: 848 - DNA, 16 bp, Homo sapiens
atggcgggcacaggct

SEQ ID NO: 849 - DNA, 15 bp, Homo sapiens
atggcgggcacaggc

SEQ ID NO: 850 - DNA, 14 bp, Homo sapiens
atggcgggcacagg

SEQ ID NO: 851 - DNA, 13 bp, Homo sapiens
atggcgggcacag

SEQ ID NO: 852 - DNA, 12 bp, Homo sapiens
atggcgggcaca

SEQ ID NO: 853 - DNA, 11 bp, Homo sapiens
atggcgggcac

SEQ ID NO: 854 - DNA, 10 bp, Homo sapiens
atggcgggca

SEQ ID NO: 855 - DNA, 19 bp, Homo sapiens
tggcgggcacaggctgggc

SEQ ID NO: 856 - DNA, 18 bp, Homo sapiens
tggcgggcacaggctggg

SEQ ID NO: 857 - DNA, 17 bp, Homo sapiens
tggcgggcacagggtgg

SEQ ID NO: 858 - DNA, 16 bp, Homo sapiens
tggcgggcacagggtg

SEQ ID NO: 859 - DNA, 15 bp, Homo sapiens
tggcgggcacagggt

SEQ ID NO: 860 - DNA, 14 bp, Homo sapiens
tggcgggcacaggg

SEQ ID NO: 861 - DNA, 13 bp, Homo sapiens
tggcgggcacagg

SEQ ID NO: 862 - DNA, 12 bp, Homo sapiens
tggcgggcacag

SEQ ID NO: 863 - DNA, 11 bp, Homo sapiens
tggcgggcaca

SEQ ID NO: 864 - DNA, 10 bp, Homo sapiens
tggcgggcac

SEQ ID NO: 865 - DNA, 18 bp, Homo sapiens
ggcgggcacagggtgggg

SEQ ID NO: 866 - DNA, 17 bp, Homo sapiens
ggcgggcacagggtggg

SEQ ID NO: 867 - DNA, 16 bp, Homo sapiens
ggcgggcacagggtgg

SEQ ID NO: 868 - DNA, 15 bp, Homo sapiens
ggcgggcacagggtg

SEQ ID NO: 869 - DNA, 14 bp, Homo sapiens
ggcgggcacagggt

SEQ ID NO: 870 - DNA, 13 bp, Homo sapiens
ggcgggcacaggg

SEQ ID NO: 871 - DNA, 12 bp, Homo sapiens
ggcgggcacagg

SEQ ID NO: 872 - DNA, 11 bp, Homo sapiens
ggcgggcacag

SEQ ID NO: 873 - DNA, 10 bp, Homo sapiens
ggcgggcaca

SEQ ID NO: 874 - DNA, 17 bp, Homo sapiens
gcgggcacagggtgggg

SEQ ID NO: 875 - DNA, 16 bp, Homo sapiens
gcgggcacagggtggg

SEQ ID NO: 876 - DNA, 15 bp, Homo sapiens
gcgggcacagggtgg

SEQ ID NO: 877 - DNA, 14 bp, Homo sapiens
gcgggcacagggtg

SEQ ID NO: 878 - DNA, 13 bp, Homo sapiens
gcgggcacagggt

SEQ ID NO: 879 - DNA, 12 bp, Homo sapiens
gcgggcacaggg

SEQ ID NO: 880 - DNA, 11 bp, Homo sapiens
gcgggcacagg

SEQ ID NO: 881 - DNA, 10 bp, Homo sapiens
gcgggcacag

SEQ ID NO: 882 - DNA, 16 bp, Homo sapiens
cgggcacaggctgggc

SEQ ID NO: 883 - DNA, 15 bp, Homo sapiens
cggggagaggctggg

SEQ ID NO: 884 - DNA, 14 bp, Homo sapiens
cggggagaggctgg

SEQ ID NO: 885 - DNA, 13 bp, Homo sapiens
cggggagaggctg

SEQ ID NO: 886 - DNA, 12 bp, Homo sapiens
cggggagaggct

SEQ ID NO: 887 - DNA, 11 bp, Homo sapiens
cggggagaggc

SEQ ID NO: 888 - DNA, 10 bp, Homo sapiens
cggggagagg

SEQ ID NO: 889 - DNA, 15 bp, Homo sapiens
gggcacaggctgggc

SEQ ID NO: 890 - DNA, 14 bp, Homo sapiens
gggcacaggctggg

SEQ ID NO: 891 - DNA, 13 bp, Homo sapiens
gggcacaggctgg

SEQ ID NO: 892 - DNA, 12 bp, Homo sapiens
gggcacaggctg

SEQ ID NO: 893 - DNA, 11 bp, Homo sapiens
gggcacaggct

SEQ ID NO: 894 - DNA, 10 bp, Homo sapiens
gggcacaggc

SEQ ID NO: 895 - DNA, 14 bp, Homo sapiens
ggcacaggctgggc

SEQ ID NO: 896 - DNA, 13 bp, Homo sapiens
ggcacaggctggg

SEQ ID NO: 897 - DNA, 12 bp, Homo sapiens
ggcacaggctgg

SEQ ID NO: 898 - DNA, 11 bp, Homo sapiens
ggcacaggctg

SEQ ID NO: 899 - DNA, 10 bp, Homo sapiens
ggcacaggct

SEQ ID NO: 900 - DNA, 13 bp, Homo sapiens
gcacaggctgggc

SEQ ID NO: 901 - DNA, 12 bp, Homo sapiens
gcacaggctggg

SEQ ID NO: 902 - DNA, 11 bp, Homo sapiens
gcacaggctgg

SEQ ID NO: 903 - DNA, 10 bp, Homo sapiens
gcacaggctg

SEQ ID NO: 904 - DNA, 12 bp, Homo sapiens
cacaggctgggc

SEQ ID NO: 905 - DNA, 11 bp, Homo sapiens
cacaggctggg

SEQ ID NO: 906 - DNA, 10 bp, Homo sapiens
cacaggctgg

SEQ ID NO: 907 - DNA, 11 bp, Homo sapiens
acaggctgggc

SEQ ID NO: 908 - DNA, 10 bp, Homo sapiens
acaggctggg

SEQ ID NO: 909 - DNA, 10 bp, Homo sapiens
caggctgggc

SEQ ID NO: 910 - DNA, 51 bp, Homo sapiens; identical to SEQ ID NO: 7

SEQ ID NO: 911 - DNA, 50 bp, Homo sapiens; identical to SEQ ID NO: 49

SEQ ID NO: 912 - DNA, 49 bp, Homo sapiens; identical to SEQ ID NO: 90

SEQ ID NO: 913 - DNA, 48 bp, Homo sapiens; identical to SEQ ID NO: 130

SEQ ID NO: 914 - DNA, 47 bp, Homo sapiens; identical to SEQ ID NO: 169

SEQ ID NO: 915 - DNA, 46 bp, Homo sapiens; identical to SEQ ID NO: 207

SEQ ID NO: 916 - DNA, 45 bp, Homo sapiens; identical to SEQ ID NO: 244

SEQ ID NO: 917 - DNA, 44 bp, Homo sapiens; identical to SEQ ID NO: 280

SEQ ID NO: 918 - DNA, 43 bp, Homo sapiens; identical to SEQ ID NO: 315

SEQ ID NO: 919 - DNA, 42 bp, Homo sapiens; identical to SEQ ID NO: 349

SEQ ID NO: 920 - DNA, 41 bp, Homo sapiens; identical to SEQ ID NO: 382

SEQ ID NO: 921 - DNA, 40 bp, Homo sapiens; identical to SEQ ID NO: 414

SEQ ID NO: 922 - DNA, 39 bp, Homo sapiens; identical to SEQ ID NO: 445

SEQ ID NO: 923 - DNA, 38 bp, Homo sapiens; identical to SEQ ID NO: 475

SEQ ID NO: 924 - DNA, 37 bp, Homo sapiens; identical to SEQ ID NO: 504

SEQ ID NO: 925 - DNA, 36 bp, Homo sapiens; identical to SEQ ID NO: 532

SEQ ID NO: 926 - DNA, 35 bp, Homo sapiens; identical to SEQ ID NO: 559

SEQ ID NO: 927 - DNA, 34 bp, Homo sapiens; identical to SEQ ID NO: 585

SEQ ID NO: 928 - DNA, 33 bp, Homo sapiens; identical to SEQ ID NO: 610

SEQ ID NO: 929 - DNA, 32 bp, Homo sapiens; identical to SEQ ID NO: 634

SEQ ID NO: 930 - DNA, 31 bp, Homo sapiens; identical to SEQ ID NO: 657

SEQ ID NO: 931 - DNA, 30 bp, Homo sapiens; identical to SEQ ID NO: 679

SEQ ID NO: 932 - DNA, 29 bp, Homo sapiens
gagggcggcatggcgggcacaggctgggc

SEQ ID NO: 933 - DNA, 28 bp, Homo sapiens
agggcggcatggcgggcacaggctgggc

SEQ ID NO: 934 - DNA, 27 bp, Homo sapiens
gggcggcatggcgggcacaggctgggc

SEQ ID NO: 935 - DNA, 26 bp, Homo sapiens
ggcggcatggcgggcacaggctgggc

SEQ ID NO: 936 - DNA, 25 bp, Homo sapiens
gcggcatggcgggcacaggctgggc

SEQ ID NO: 937 - DNA, 24 bp, Homo sapiens
cggcatggcgggcacaggctgggc

SEQ ID NO: 938 - DNA, 23 bp, Homo sapiens
ggcatggcgggcacaggctgggc

SEQ ID NO: 939 - DNA, 22 bp, Homo sapiens
gcatggcgggcacaggctgggc

SEQ ID NO: 940 - DNA, 21 bp, Homo sapiens
catggcgggcacaggctgggc

SEQ ID NO: 941 - DNA, 20 bp, Homo sapiens
atggcgggcacaggctgggc

SEQ ID NO: 942 - DNA, 19 bp, Homo sapiens
tggcgggcacaggctgggc

SEQ ID NO: 943 - DNA, 18 bp, Homo sapiens
ggcgggcacaggctgggc

SEQ ID NO: 944 - DNA, 17 bp, Homo sapiens
gcgggcacaggctgggc

SEQ ID NO: 945 - DNA, 16 bp, Homo sapiens
cgggcacaggctgggc

SEQ ID NO: 946 - DNA, 15 bp, Homo sapiens
gggcacaggctgggc

SEQ ID NO: 947 - DNA, 14 bp, Homo sapiens
ggcacaggctgggc

SEQ ID NO: 948 - DNA, 13 bp, Homo sapiens
gcacaggctgggc

SEQ ID NO: 949 - DNA, 12 bp, Homo sapiens
cacaggctgggc

SEQ ID NO: 950 - DNA, 11 bp, Homo sapiens
acaggctgggc

SEQ ID NO: 951 - DNA, 10 bp, Homo sapiens
caggctgggc

SEQ ID NO: 952 - DNA, 9 bp, Homo sapiens
aggctgggc

SEQ ID NO: 953 - DNA, 15 bp, Homo sapiens
ttttccttcctttgt

SEQ ID NO: 954 - DNA, 23 bp, Homo sapiens
ctctcttcgctcccggctgcctg

SEQ ID NO: 955 - DNA, 15 bp, Homo sapiens
ctcggccgtgcggct

SEQ ID NO: 956 - DNA, 29 bp, Homo sapiens
ctgtcgctcccggtccgccgccctccggg

SEQ ID NO: 957 - DNA, 20 bp, Homo sapiens
gggtctgctgccgttggctg

SEQ ID NO: 958 - DNA, 18 bp, Homo sapiens
ccccttctgcgggtcgcc

SEQ ID NO: 959 - DNA, 17 bp, Homo sapiens
ggtgctgggcttgtggc

SEQ ID NO: 960 - DNA, 15 bp, Homo sapiens
ggcctctcttctggg

SEQ ID NO: 961 - DNA, 14 bp, Homo sapiens
cctggtccctccgt

SEQ ID NO: 962 - DNA, 14 bp, Homo sapiens
ggtggctcctctgc

SEQ ID NO: 963 - DNA, 15 bp, Homo sapiens
gcttggtcctggggc

SEQ ID NO: 964 - DNA, 18 bp, Homo sapiens
tgctgctctcctctcctt

SEQ ID NO: 965 - DNA, 15 bp, Homo sapiens
tgcttttcttttctg

SEQ ID NO: 966 - DNA, 21 bp, Homo sapiens
ggcctctgtggtctgtttttt

SEQ ID NO: 967 - DNA, 19 bp, Homo sapiens
tctggccctgctggggcgc

SEQ ID NO: 968 - DNA, 20 bp, Homo sapiens
tctccgccgcccgcctggct

SEQ ID NO: 969 - DNA, 18 bp, Homo sapiens
cccggbgcccbtgbtggg

SEQ ID NO: 970 - DNA, 21 bp, Homo sapiens
cbtgccgtggttcttgccctc

SEQ ID NO: 971 - DNA, 24 bp, Homo sapiens
ctttggctgccgtgcccgctcccc

SEQ ID NO: 972 - DNA, 15 bp, Homo sapiens
ggcctcctggcgggt

SEQ ID NO: 973 - DNA, 20 bp, Homo sapiens
cgttgggcccgtgttcccct

SEQ ID NO: 974 - DNA, 18 bp, Homo sapiens
ggggcctggggctccctt

SEQ ID NO: 975 - DNA, 20 bp, Homo sapiens
ctctcgcccttcttgctggg

SEQ ID NO: 976 - DNA, 19 bp, Homo sapiens
cctctgctgctgctggtgc

SEQ ID NO: 977 - DNA, 32 bp, Homo sapiens
tgtggcccccgtacaccgaggagcccatgatg

SEQ ID NO: 978 - DNA, 43 bp, Homo sapiens
ggcatgccacagacgacaggcgtbcbccgbggbgcccbtgbtg

SEQ ID NO: 979 - DNA, 50 bp, Homo sapiens
ggcbtgccbcbgbcgbcbggcggcgccgtgccgcgtcttggtggcggcgg

SEQ ID NO: 980 - DNA, 30 bp, Homo sapiens
gttcgcgcccgcgcggggcccctccggtcc

SEQ ID NO: 981 - DNA, 35 bp, Homo sapiens
ttggcccgcgcgcccgcccgtctcgggctgggcgg

SEQ ID NO: 982 - DNA, 22 bp, Homo sapiens
cgggtcggggccccccgcggcc

SEQ ID NO: 983 - DNA, 29 bp, Homo sapiens
gcctcggggctggggcgctggtggccggg

SEQ ID NO: 984 - DNA, 24 bp, Homo sapiens
ccgcgcctccgcctgccgcttctg

SEQ ID NO: 985 - DNA, 21 bp, Homo sapiens
gctgggccccgggcgccccct

SEQ ID NO: 986 - DNA, 23 bp, Homo sapiens
cccctcttgctcgggtccccgtg

SEQ ID NO: 987 - DNA, 48 bp, Homo sapiens
acagcgcgtcctgtgtctccagcagcatggccgggccagctgggcccc

SEQ ID NO: 988 - DNA, 48 bp, Homo sapiens
bcbgcgcgtcctgtgtctccbgcbgcbtggccgggccbgctgggcccc

SEQ ID NO: 989 - DNA, 39 bp, Homo sapiens
acagagcatgctgttgttgggcatcttgccttcccaggg

SEQ ID NO: 990 - DNA, 39 bp, Homo sapiens
bcbgbgcbtgctgttgttgggcbtcttgccttcccbggg

SEQ ID NO: 991 - DNA, 18 bp, Homo sapiens
cccttttctggtggggtg

SEQ ID NO: 992 - DNA, 15 bp, Homo sapiens
gtgctgttgttgggc

SEQ ID NO: 993 - DNA, 14 bp, Homo sapiens
tttcttctgttccc

SEQ ID NO: 994 - DNA, 18 bp, Homo sapiens
cccttttctggtggggtg

SEQ ID NO: 995 - DNA, 15 bp, Homo sapiens
gtgctgttgttgggc

SEQ ID NO: 996 - DNA, 14 bp, Homo sapiens
tttcttctgttccc